(19) United States
(12) Patent Application Publication
(10) Pub. No.: US 2009/0023731 A1
(43) Pub. Date: Jan. 22, 2009

(54) SOLUBLE EPOXIDE HYDROLASE INHIBITORS

(75) Inventors: Richard D. Gless, JR., Oakland, CA (US); Sampath Kumar Anandan, Fremont, CA (US)

Correspondence Address: FOLEY & LARDNER LLP, 975 PAGE MILL ROAD, PALO ALTO, CA, 94304, US

(73) Assignee: Arete Therapeutics, Inc.

(21) Appl. No.: 12/052,966

(22) Filed: Mar. 21, 2008

Related U.S. Application Data
(60) Provisional application No. 60/896,421, filed on Mar. 22, 2007.
(60) Provisional application No. 60/971,508, filed on Sep. 11, 2007.
(60) Provisional application No. 60/972,169, filed on Sep. 13, 2007.

(51) Int. Cl. A61K 31/5377; C07D 265/30; C07D 413/02; C07C 275/00; A61K 31/5375; A61K 31/17; A61P 9/12; A61P 19/02; A61P 11/06

(52) U.S. Cl. 514/235.5; 544/168; 514/237.8; 514/238.8; 544/170; 544/131; 564/48; 514/596

(57) ABSTRACT

Disclosed are urea and thiourea compounds and compositions that inhibit soluble epoxide hydrolase (sEH), methods for preparing the compounds and compositions, and methods for treating patients with such compounds and compositions. The compounds, compositions, and methods are useful for treating a variety of sEH mediated diseases, including hypertensive, cardiovascular, inflammatory, and diabetic-related diseases.

### CROSS-REFERENCE TO RELATED APPLICATIONS

This application claims the benefit under 35 U.S.C. § 119(e) of provisional Patent Application Ser. No. 60/896,421, filed on Mar. 22, 2007, provisional Patent Application Ser. No. 60/971,508, filed on Sep. 11, 2007, and provisional Patent Application Ser. No. 60/972,169, filed on Sep. 13, 2007, all of which are incorporated herein by reference in their entirety.

### BACKGROUND OF THE INVENTION

1. Field of the Invention

This invention relates to the field of pharmaceutical chemistry. Provided herein are amide, urea, and thiourea compounds that inhibit soluble epoxide hydrolase (sEH), pharmaceutical compositions containing such compounds, methods for preparing the compounds and formulations, and methods for treating patients with such compounds and compositions. The compounds, compositions, and methods are useful for treating a variety of sEH mediated diseases, including hypertensive, cardiovascular, inflammatory, metabolic syndrome, and diabetic-related diseases.

2. State of the Art

The arachidonate cascade is a ubiquitous lipid signaling cascade in which arachidonic acid is liberated from the plasma membrane lipid reserves in response to a variety of extra-cellular and/or intra-cellular signals. The released arachidonic acid is then available to act as a substrate for a variety of oxidative enzymes that convert arachidonic acid to signaling lipids that play critical roles in, for example, inflammation. Disruption of the pathways leading to the lipids remains an important strategy for many commercial drugs used to treat a multitude of inflammatory disorders. For example, non-steroidal anti-inflammatory drugs (NSAIDs) disrupt the conversion of arachidonic acid to prostaglandins by inhibiting cyclooxygenases (COX1 and COX2). New asthma drugs, such as SINGULAIR™ disrupt the conversion of arachidonic acid to leukotrienes by inhibiting lipoxygenase (LOX).

Certain cytochrome P450-dependent enzymes convert arachidonic acid into a series of epoxide derivatives known as epoxyeicosatrienoic acids (EETs). These EETs are particularly prevalent in endothelium (cells that make up arteries and vascular beds), kidney, and lung. In contrast to many of the end products of the prostaglandin and leukotriene pathways, the EETs have a variety of anti-inflammatory and anti-hypertensive properties and are known to be potent vasodilators and mediators of vascular permeability.

While EETs have potent effects in vivo, the epoxide moiety of the EETs is rapidly hydrolyzed into the less active dihydroxyeicosatrienoic acid (DHET) form by an enzyme called soluble epoxide hydrolase (sEH). Inhibition of sEH has been found to significantly reduce blood pressure in hypertensive animals (see, e.g., Yu et al. Circ. Res. 87:992-8 (2000) and Sinal et al. J. Biol. Chem. 275:40504-10 (2000)), to reduce the production of proinflammatory nitric oxide (NO), cytokines, and lipid mediators, and to contribute to inflammatory resolution by enhancing lipoxin A₄ production in vivo (see. Schmelzer et al. Proc. Nat'l Acad. Sci. USA 102(28):9772-7 (2005)).

Various small molecule compounds have been found to inhibit sEH and elevate EET levels (Morisseau et al. Annu. Rev. Pharmacol. Toxicol. 45:311-33 (2005)). The availability of more potent compounds capable of inhibiting sEH and its inactivation of EETs would be highly desirable for treating a wide range of disorders that arise from inflammation and hypertension or that are otherwise mediated by sEH.

### SUMMARY OF THE INVENTION

This invention relates to compounds and their pharmaceutical compositions, to their preparation, and to their uses for treating diseases mediated by soluble epoxide hydrolase (sEH). In accordance with one aspect of the invention, provided are compounds having Formula (I′) or a pharmaceutically acceptable salt thereof:

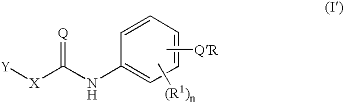

wherein:
Q is O or S;
Q′ is O or S;
R is selected from the group consisting of substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3;
X is selected from the group consisting of a covalent bond, NH, or CR′R″ where R′ and R″ are independently H or alkyl or R′ and R″ together form a C₃-C₆ cycloalkyl ring; and
Y is selected from the group consisting of heteroaryl, substituted heteroaryl, and

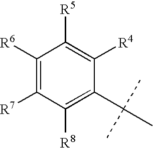

wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring;
provided that
(1) if X is NH and Q is O, then R is not pyridyl, piperidinyl, or piperidinyl substituted with at least one substituent selected from the group consisting of —C(O)H, —C(O)CH₃, —C(O)Oalkyl, —C(O)N(CH₃)₂, dimethylamino, cyanoimino-morpholin-4-yl-methyl, N¹-azetidin-1-yl-N²-cyano-amidino, N²-cyano-N¹,N¹-dimethylamidino, N′-cyano-N,N-dimethyl-carbamimidoyl, propionyl, and methylsulfonyl;
(2) if X is NH, Q is O, and Y is methoxyphenyl, then R is not hydroxymethylphenyl, pyridylalkyl, fluoropyridyl, and acetylphenyl;
(3) if Y is pyridyl or substituted pyridyl, then R is alkyl substituted with NR²R³ wherein R² and R³ together form a morpholino or piperazinyl ring;
(4) R is not haloalkyl or mono-substituted alkyl where the substituent is cyano, hydroxyl, or —O—C(O)O-alkyl;
(5) when Y is phenyl, substituted phenyl, heteroaryl or substituted heteroaryl, R is not heteroaryl selected from the group consisting of benzimidazolyl, benzothiazolyl, benzoxazolyl, diazaindolinyl, pyridoimidazolyl, azaindolizinyl, 3,4-diazaindolyl, azaindolyl, 3,4-dihydro-1,4a,5-triazacarbazolonyl, and 3,4-dihydro-1,4a-diazacarbazolonyl, wherein the heteroaryl is substituted with at least one substituent selected from the group consisting of amino, (carboxyl ester)amino, acylamino, (substituted sulfonyl)amino, substituted sulfonyl, aminosulfonylamino, and aminocarbonylamino; and
(6) Formula (I′) is not

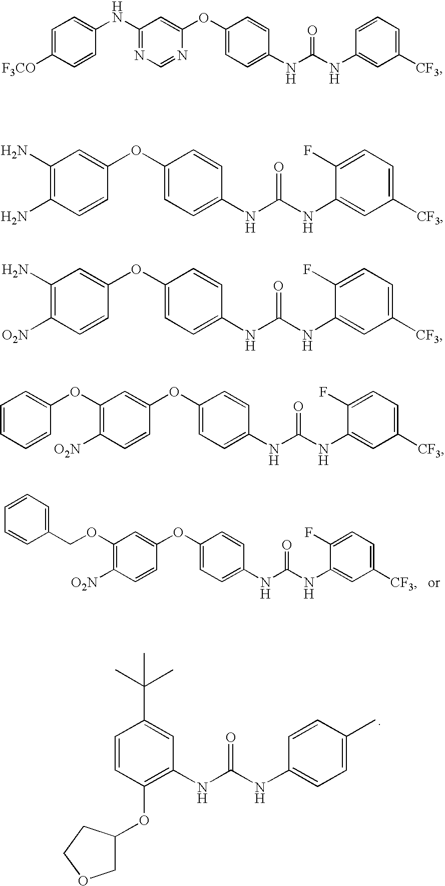

In one embodiment, provided are compounds having Formula (I) or a pharmaceutically acceptable salt thereof:

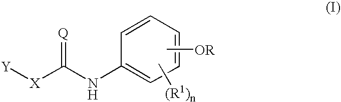

wherein:
Q is O or S;
R is selected from the group consisting of substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3;
X is selected from the group consisting of a covalent bond, NH, or CR′R″ where R′ and R″ are independently H or alkyl or R′ and R″ together form a C₃-C₆ cycloalkyl ring;
Y is selected from the group consisting of heteroaryl, substituted heteroaryl, and

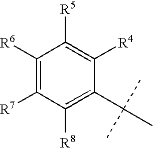

wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring;

provided that
(1) if X is NH and Q is O, then R is not pyridyl, piperidinyl, or piperidinyl substituted with at least one substituent selected from the group consisting of —C(O)H, —C(O)CH₃, —C(O)Oalkyl, —C(O)N(CH₃)₂, dimethylamino, cyanoimino-morpholin-4-yl-methyl, N¹-azetidin-1-yl-N²-cyano-amidino, N²-cyano-N¹, N¹-dimethylamidino, N′-cyano-N,N-dimethyl-carbamimidoyl, propionyl, and methylsulfonyl;
(2) if X is NH, Q is O, and Y is methoxyphenyl, then R is not hydroxymethylphenyl, pyridylalkyl, fluoropyridyl, and acetylphenyl;
(3) if Y is pyridyl or substituted pyridyl, then R is alkyl substituted with NR²R³ wherein R and R³ together form a morpholino or piperazinyl ring;
(4) R is not haloalkyl or mono-substituted alkyl where the substituent is cyano, hydroxyl, or —O—C(O)O-alkyl;
(5) when Y is phenyl, substituted phenyl, heteroaryl or substituted heteroaryl, R is not heteroaryl selected from the group consisting of benzimidazolyl, benzothiazolyl, benzoxazolyl, diazaindolinyl, pyridoimidazolyl, azaindolizinyl, 3,4-diazaindolyl, azaindolyl, 3,4-dihydro-1,4-a,5-triazacarbazolonyl, and 3,4-dihydro-1,4-a-diazacarbazolonyl, wherein the heteroaryl is substituted with at least one substituent selected from the group consisting of amino, (carboxyl ester)amino, acylamino, (substituted sulfonyl)amino, substituted sulfonyl, aminosulfonylamino, and aminocarbonylamino;

and
(6) Formula (I) is not

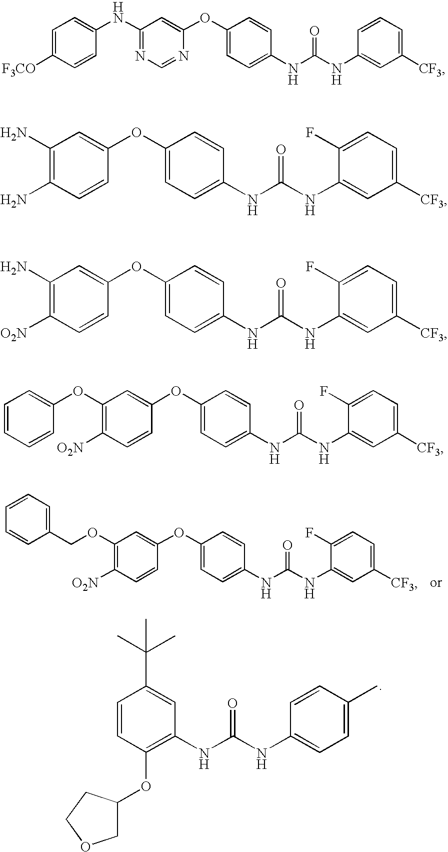

In another embodiment, provided are compounds having Formula (Ia) or (Ib), or a pharmaceutically acceptable salt thereof:

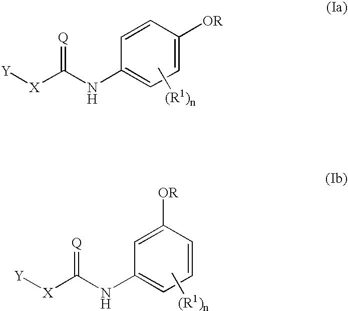

wherein:
Q is O or S;
X is selected from the group consisting of a covalent bond, NH, or CH₂;
R is selected from the group consisting of substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3; and
Y is selected from the group consisting of pyridyl, substituted pyridyl, and

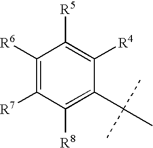

wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring;

provided that
(1) if X is NH and Q is O, then R is not pyridyl, piperidinyl, or piperidinyl substituted with at least one substituent selected from the group consisting of —C(O)H, —C(O)CH₃, —C(O)Oalkyl, —C(O)N(CH₃)₂, dimethylamino, cyanoimino-morpholin-4-yl-methyl, N¹-azetidin-1-yl-N²-cyano-amidino, N²-cyano-N¹,N¹-dimethylamidino, N′-cyano-N,N-dimethyl-carbamimidoyl, propionyl, and methylsulfonyl;
(2) if X is NH, Q is O, and Y is methoxyphenyl, then R is not hydroxymethylphenyl, pyridylalkyl, fluoropyridyl, and acetylphenyl;
(3) if Y is pyridyl or substituted pyridyl, then R is alkyl substituted with NR²R³ wherein R² and R³ together form a morpholino or piperazinyl ring;
(4) R is not haloalkyl or mono-substituted alkyl where the substituent is cyano, hydroxyl, or —O—C(O)O-alkyl;
(5) when Y is phenyl, substituted phenyl, heteroaryl or substituted heteroaryl, R is not heteroaryl selected from the group consisting of benzimidazolyl, benzothiazolyl, benzoxazolyl, diazaindolinyl, pyridoimidazolyl, azaindolizinyl, 3,4-diazaindolyl, azaindolyl, 3,4-dihydro-1,4-a,5-triazacarbazolonyl, and 3,4-dihydro-1,4-a-diazacarbazolonyl, wherein the heteroaryl is substituted with at least one substituent selected from the group consisting of amino, (carboxyl ester)amino, acylamino, (substituted sulfonyl)amino, substituted sulfonyl, aminosulfonylamino, and aminocarbonylamino; and
(6) Formula (Ia) is not

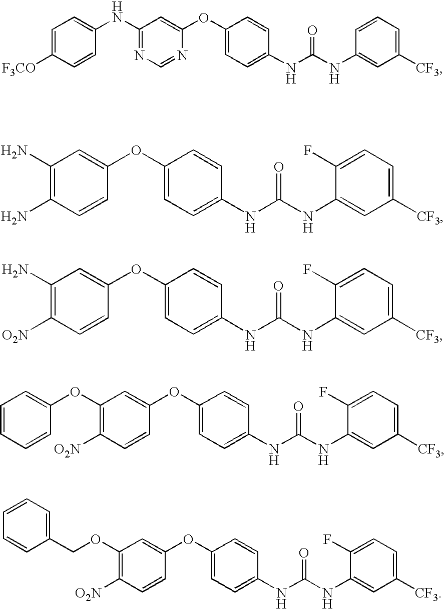

In another embodiment, provided are compounds of Table 1 or 2 or a pharmaceutically acceptable salt thereof.

In accordance with another aspect of the invention, provided is a method for treating a soluble expoxide hydrolase mediated disease, said method comprising administering to a patient a pharmaceutical composition comprising a pharmaceutically acceptable carrier and a therapeutically effective amount of a compound of the invention or a pharmaceutically acceptable salt thereof.

These and other embodiments of the present invention are further described in the text that follows.

### DETAILED DESCRIPTION OF THE INVENTION

### Definitions

As used herein, the following definitions shall apply unless otherwise indicated.

“cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The amino acid sequence of human sEH is also set forth as SEQ ID NO:2 of U.S. Pat. No. 5,445,956; the nucleic acid sequence encoding the human sEH is set forth as nucleotides 42-1703 of SEQ ID NO:1 of that patent. The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

“Chronic Obstructive Pulmonary Disease” or “COPD” is also sometimes known as “chronic obstructive airway disease”, “chronic obstructive lung disease”, and “chronic airways disease.” COPD is generally defined as a disorder characterized by reduced maximal expiratory flow and slow forced emptying of the lungs. COPD is considered to encompass two related conditions, emphysema and chronic bronchitis. COPD can be diagnosed by the general practitioner using art recognized techniques, such as the patient's forced vital capacity (“FVC”), the maximum volume of air that can be forcibly expelled after a maximal inhalation. In the offices of general practitioners, the FVC is typically approximated by a 6 second maximal exhalation through a spirometer. The definition, diagnosis and treatment of COPD, emphysema, and chronic bronchitis are well known in the art and discussed in detail by, for example, Honig and Ingram, in Harrison's Principles of Internal Medicine, (Fauci et al., Eds), 14th Ed., 1998, McGraw-Hill, New York, pp. 1451-1460 (hereafter, “Harrison's Principles of Internal Medicine”). As the names imply, “obstructive pulmonary disease” and “obstructive lung disease” refer to obstructive diseases, as opposed to restrictive diseases. These diseases particularly include COPD, bronchial asthma, and small airway disease.

“Emphysema” is a disease of the lungs characterized by permanent destructive enlargement of the airspaces distal to the terminal bronchioles without obvious fibrosis.

“Chronic bronchitis” is a disease of the lungs characterized by chronic bronchial secretions which last for most days of a month, for three months, a year, for two years, etc.

“Small airway disease” refers to diseases where airflow obstruction is due, solely or predominantly to involvement of the small airways. These are defined as airways less than 2 mm in diameter and correspond to small cartilaginous bronchi, terminal bronchioles, and respiratory bronchioles. Small airway disease (SAD) represents luminal obstruction by inflammatory and fibrotic changes that increase airway resistance. The obstruction may be transient or permanent.

“Interstitial lung diseases (ILDs)” are restrictive lung diseases involving the alveolar walls, perialveolar tissues, and contiguous supporting structures. As discussed on the website of the American Lung Association, the tissue between the air sacs of the lung is the interstitium, and this is the tissue affected by fibrosis in the disease. Persons with such restrictive lung disease have difficulty breathing in because of the stiffness of the lung tissue but, in contrast to persons with obstructive lung disease, have no difficulty breathing out. The definition, diagnosis and treatment of interstitial lung diseases are well known in the art and discussed in detail by, for example, Reynolds, H. Y., in Harrison's Principles of Internal Medicine, supra, at pp. 1460-1466. Reynolds notes that, while ILDs have various initiating events, the immunopathological responses of lung tissue are limited and the ILDs therefore have common features.

“Idiopathic pulmonary fibrosis,” or “IPF,” is considered the prototype ILD. Although it is idiopathic in that the cause is not known, Reynolds, supra, notes that the term refers to a well defined clinical entity.

“Bronchoalveolar lavage,” or “BAL,” is a test which permits removal and examination of cells from the lower respiratory tract and is used in humans as a diagnostic procedure for pulmonary disorders such as IPF. In human patients, it is usually performed during bronchoscopy.

“Diabetic neuropathy” refers to acute and chronic peripheral nerve dysfunction resulting from diabetes.

“Diabetic nephropathy” refers to renal diseases resulting from diabetes.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NRC(O)alkyl, —NRC(O)substituted alkyl, —NRC(O)cycloalkyl, —NRC(O)substituted cycloalkyl, —NRC(O)cycloalkenyl, —NRC(O)substituted cycloalkenyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic wherein R is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR¹⁵R¹⁶ where R¹⁵ and R¹⁶ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R¹⁵ and R¹⁶ are optionally joined, together with the nitrogen bound thereto to form a heterocyclic or substituted heterocyclic group, provided that R¹⁵ and R¹⁶ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R¹⁵ is hydrogen and R¹⁶ is alkyl, the substituted amino group is sometimes referred to herein as alkylamino. When R¹⁵ and R¹⁶ are alkyl, the substituted amino group is sometimes referred to herein as dialkylamino. When referring to a monosubstituted amino, it is meant that either R¹⁵ or R¹⁶ is hydrogen but not both. When referring to a disubstituted amino, it is meant that neither R¹⁵ nor R¹⁶ are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NRC(O)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NRC(S)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR¹¹R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR¹¹R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR—SO₂NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR¹²)NR¹⁰R¹¹ where R¹⁰, R¹¹, and R¹² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR—C(O)O-alkyl, —NR—C(O)O-substituted alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic wherein R is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, substituted —O—C(O)O-alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spirobicyclo groups such as spiro[4.5]dec-8-yl:

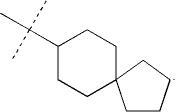

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR¹³C(═NR¹³)N(R¹³)₂ where each R¹³ is independently selected from the group consisting of hydrogen, alkyl, substituted alkyl, aryl, substituted aryl, heteroaryl, substituted heteroaryl, heterocyclic, and substituted heterocyclic and two R¹³ groups attached to a common guanidino nitrogen atom are optionally joined together with the nitrogen bound thereto to form a heterocyclic or substituted heterocyclic group, provided that at least one R¹³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups and having no other substituents, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocycyl.

“Substituted heterocyclyloxy” refers to the group —O-(substituted heterocycyl).

“Heterocyclylthio” refers to the group —S-heterocycyl.

“Substituted heterocyclylthio” refers to the group —S-(substituted heterocycyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “haloalkylsulfonyl” refers to —SO₂-haloalkyl where haloalkyl is defined herein. The term “(substituted sulfonyl)amino” refers to —NH(substituted sulfonyl) wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

“Compound” or “compounds” as used herein is meant to include the stereoiosmers and tautomers of the indicated formulas.

“Stereoisomer” or “stereoisomers” refer to compounds that differ in the chirality of one or more stereocenters. Stereoisomers include enantiomers and diastereomers.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Patient” refers to mammals and includes humans and non-human mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

“Treating” or “treatment” of a disease in a patient refers to (1) preventing the disease from occurring in a patient that is predisposed or does not yet display symptoms of the disease; (2) inhibiting the disease or arresting its development; or (3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly, in one aspect, this invention provides a compound of Formula (I′) or a pharmaceutically acceptable salt thereof:

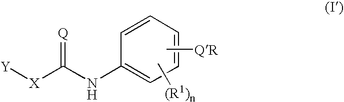

wherein:
Q is O or S;
Q′ is O or S;
R is selected from the group consisting of substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3;
X is selected from the group consisting of a covalent bond, NH, or CR′R″ where R′ and R″ are independently H or alkyl or R′ and R″ together form a C₃-C₆ cycloalkyl ring; and
Y is selected from the group consisting of heteroaryl, substituted heteroaryl, and

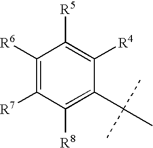

wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring;

provided that
(1) if X is NH and Q is O, then R is not pyridyl, piperidinyl, or piperidinyl substituted with at least one substituent selected from the group consisting of —C(O)H, —C(O)CH₃, —C(O)Oalkyl, —C(O)N(CH₃)₂, dimethylamino, cyanoimino-morpholin-4-yl-methyl, N¹-azetidin-1-yl-N²-cyano-amidino, N²-cyano-N¹,N¹-dimethylamidino, N′-cyano-N,N-dimethyl-carbamimidoyl, propionyl, and methylsulfonyl;
(2) if X is NH, Q is O, and Y is methoxyphenyl, then R is not hydroxymethylphenyl, pyridylalkyl, fluoropyridyl, and acetylphenyl;
(3) if Y is pyridyl or substituted pyridyl, then R is alkyl substituted with NR²R³ wherein R² and R³ together form a morpholino or piperazinyl ring;
(4) R is not haloalkyl or mono-substituted alkyl where the substituent is cyano, hydroxyl, or —O—C(O)O-alkyl;
(5) when Y is phenyl, substituted phenyl, heteroaryl or substituted heteroaryl, R is not heteroaryl selected from the group consisting of benzimidazolyl, benzothiazolyl, benzoxazolyl, diazaindolinyl, pyridoimidazolyl, azaindolizinyl, 3,4-diazaindolyl, azaindolyl, 3,4-dihydro-1,4-a,5-triazacarbazolonyl, and 3,4-dihydro-1,4-a-diazacarbazolonyl, wherein the heteroaryl is substituted with at least one substituent selected from the group consisting of amino, (carboxyl ester)amino, acylamino, (substituted sulfonyl)amino, substituted sulfonyl, aminosulfonylamino, and aminocarbonylamino;

and
(6) Formula (I′) is not

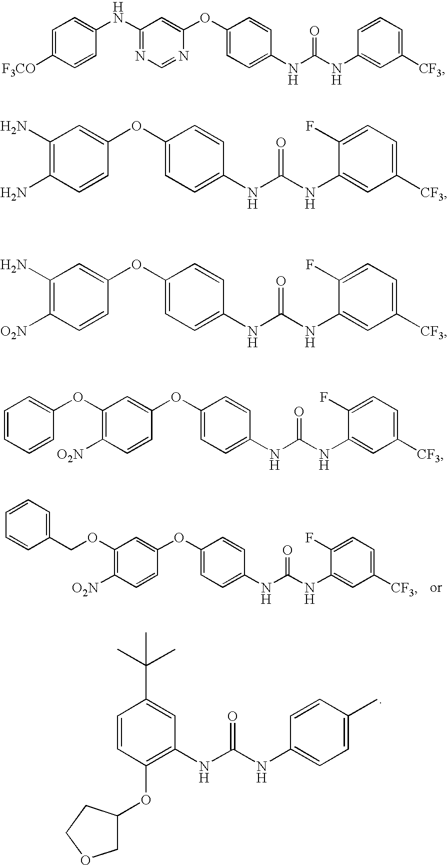

In one embodiment, provided is a compound of Formula (I) or a pharmaceutically acceptable salt thereof:

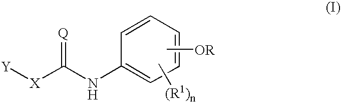

wherein:
Q is O or S;
R is selected from the group consisting of substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3;
X is selected from the group consisting of a covalent bond, NH, or CR′R″ where R′ and R″ are independently H or alkyl or R′ and R″ together form a C₃-C₆ cycloalkyl ring; and
Y is selected from the group consisting of heteroaryl, substituted heteroaryl, and

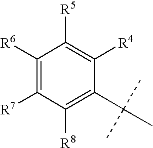

wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring;

provided that
(1) if X is NH and Q is O, then R is not pyridyl, piperidinyl, or piperidinyl substituted with at least one substituent selected from the group consisting of —C(O)H, —C(O)CH₃, —C(O)Oalkyl, —C(O)N(CH₃)₂, dimethylamino, cyanoimino-morpholin-4-yl-methyl, N¹-azetidin-1-yl-N²-cyano-amidino, N²-cyano-N¹,N¹-dimethylamidino, N′-cyano-N,N-dimethyl-carbamimidoyl, propionyl, and methylsulfonyl;
(2) if X is NH, Q is O, and Y is methoxyphenyl, then R is not hydroxymethylphenyl, pyridylalkyl, fluoropyridyl, and acetylphenyl;
(3) if Y is pyridyl or substituted pyridyl, then R is alkyl substituted with NR²R³ wherein R² and R³ together form a morpholino or piperazinyl ring;
(4) R is not haloalkyl or mono-substituted alkyl where the substituent is cyano, hydroxyl, or —O—C(O)O-alkyl;
(5) when Y is phenyl, substituted phenyl, heteroaryl or substituted heteroaryl, R is not heteroaryl selected from the group consisting of benzimidazolyl, benzothiazolyl, benzoxazolyl, diazaindolinyl, pyridoimidazolyl, azaindolizinyl, 3,4-diazaindolyl, azaindolyl, 3,4-dihydro-1,4-a,5-triazacarbazolonyl, and 3,4-dihydro-1,4-a-diazacarbazolonyl, wherein the heteroaryl is substituted with at least one substituent selected from the group consisting of amino, (carboxyl ester)amino, acylamino, (substituted sulfonyl)amino, substituted sulfonyl, aminosulfonylamino, and aminocarbonylamino; and
(6) Formula (I) is not

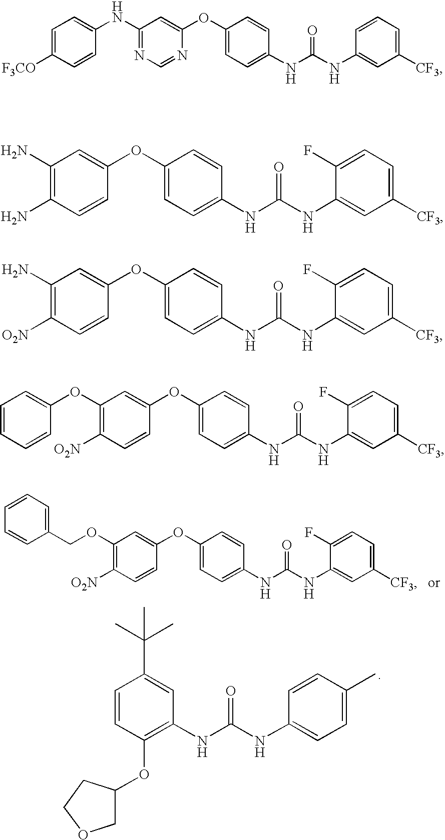

In another embodiment, provided is a compound of Formula (Ia) or (Ib) or a pharmaceutically acceptable salt thereof:

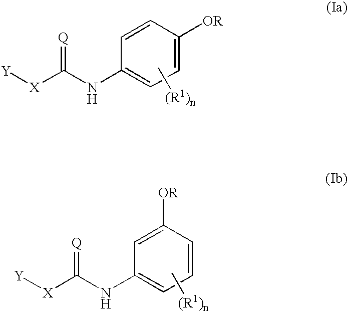

wherein:
Q is O or S;
X is selected from the group consisting of a covalent bond, NH, or CH₂;
R is selected from the group consisting of substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3; and
Y is selected from the group consisting of pyridyl, substituted pyridyl, and

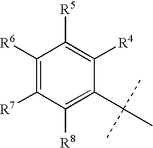

wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring;

provided that
(1) if X is NH and Q is O, then R is not pyridyl, piperidinyl, or piperidinyl substituted with at least one substituent selected from the group consisting of —C(O)H, —C(O)CH₃, —C(O)Oalkyl, —C(O)N(CH₃)₂, dimethylamino, cyanoimino-morpholin-4-yl-methyl, N¹-azetidin-1-yl-N²-cyano-amidino, N²-cyano-N¹,N¹-dimethylamidino, N′-cyano-N,N-dimethyl-carbamimidoyl, propionyl, and methylsulfonyl;
(2) if X is NH, Q is O, and Y is methoxyphenyl, then R is not hydroxymethylphenyl, pyridylalkyl, fluoropyridyl, and acetylphenyl;
(3) if Y is pyridyl or substituted pyridyl, then R is alkyl substituted with NR²R³ wherein R² and R³ together form a morpholino or piperazinyl ring;
(4) R is not haloalkyl or mono-substituted alkyl where the substituent is cyano, hydroxyl, or —O—C(O)O-alkyl;
(5) when Y is phenyl, substituted phenyl, heteroaryl or substituted heteroaryl, R is not heteroaryl selected from the group consisting of benzimidazolyl, benzothiazolyl, benzoxazolyl, diazaindolinyl, pyridoimidazolyl, azaindolizinyl, 3,4-diazaindolyl, azaindolyl, 3,4-dihydro-1,4-a,5-triazacarbazolonyl, and 3,4-dihydro-1,4-a-diazacarbazolonyl, wherein the heteroaryl is substituted with at least one substituent selected from the group consisting of amino, (carboxyl ester)amino, acylamino, (substituted sulfonyl)amino, substituted sulfonyl, aminosulfonylamino, and aminocarbonylamino;

and
(6) Formula (Ia) is not

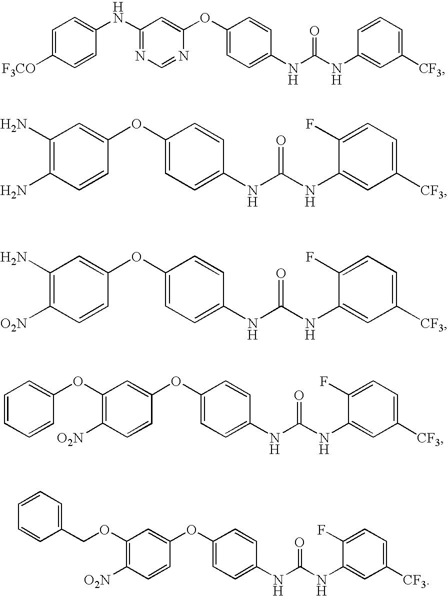

Various embodiments relating to the compounds or pharmaceutically acceptable salts of Formulas (I′), (I), (Ia), and (Ib) are listed below. These embodiments can be combined with each other or with any other embodiments described in this application. In some aspects, provided are compounds of Formula (I′), (I), (Ia), or (Ib) having one or more of the following features. In some embodiments of Formula (I′), (I), (Ia), or (Ib), X is NH. In some embodiments of Formula (Ia) or (Ib), X is CH₂. In some embodiments of Formula (Ia) or (Ib), X is a covalent bond.

In some embodiments Q is O. In some embodiments, Q is S.

In some embodiments of Formula (I′), Q′ is O. In some embodiments, Q′ is S.

In some embodiments R is substituted alkyl. In some embodiments, R is alkyl substituted with aryl, heterocycloalkyl, or substituted heterocycloalkyl. In some embodiments, R is benzyl. In some embodiments, R is alkyl substituted with NR²R³ wherein R² and R³ together form a morpholino or piperazinyl ring, wherein said ring may be substituted or unsubstituted.

In some embodiments R is phenyl or substituted phenyl. In some embodiments, the phenyl is substituted with —C(O)OH.

In some embodiments n is 0.

In some embodiments n is 1 and R¹ is halo. In some embodiments, R¹ is fluoro.

In some embodiments Y is pyridyl or substituted pyridyl. In some embodiments, Y is substituted or unsubstituted 2-pyridyl, 3-pyridyl, or 4-pyridyl. In still other aspects Y is 2-pyridyl, 3-pyridyl, or 4-pyridyl substituted with one to four substituents independently selected from halo, alkyl, haloalkyl, haloalkoxy, alkylthio, haloalkylthio, cyano, alkylsulfonyl, and haloalkylsulfonyl. In other embodiments the pyridyl groups are substituted with one to four substitutents independently selected from halo, trifluoromethyl, trifluoromethoxy, alkylsulfonyl, and haloalkylsulfonyl.

In some embodiments, Y is pyridyl or substituted pyridyl, R is alkyl substituted with NR²R³ wherein R² and R³ together form a morpholino or piperazinyl ring.

In some embodiments of Formula (I′), (I), (Ia), or (Ib), Y is

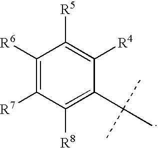

In some embodiments R⁴ and R⁸ are hydrogen.

In some embodiments at least one of R⁴ and R⁸ is fluoro or chloro. In some embodiments one of R⁴ and R⁸ is fluoro, and the other of R⁴ and R⁸ is hydrogen. In some embodiments, both R⁴ and R⁸ are fluoro or chloro.

In some embodiments R⁵, R⁶, and R⁷ are independently selected from the group consisting of hydrogen, halo, alkyl, haloalkyl, haloalkoxy, alkylamino, heterocycloalkyloxy, alkylthio, haloalkylthio, cyano, alkylsulfonyl, and haloalkylsulfonyl.

In some embodiments at least one of R⁵, R⁶, and R⁷ is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl.

In some embodiments one of R⁵, R⁶, and R⁷ is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl, and the remainder of R⁵,R⁶, and R⁷ are hydrogen.

In some embodiments at least one of R⁵, R⁶, and R⁷ is selected from the group consisting of halo, trifluoromethyl, trifluoromethoxy, alkylsulfonyl, and haloalkylsulfonyl.

In some embodiments R⁶ is selected from the group consisting of chloro, fluoro, trifluoromethyl, and trifluoromethoxy. In some embodiments, R⁴, R⁵, R⁷, and R⁸ are hydrogen.

In some embodiments R⁵ is selected from the group consisting of chloro, fluoro, trifluoromethyl, and trifluoromethoxy and R⁴, R⁶, R⁷, and R⁸ are hydrogen.

In some embodiments, R⁶ and R⁷ together form a heterocycloalkyl ring. In some embodiments, Y is

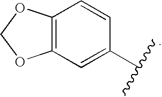

In other embodiments, provided is a compound or pharmaceutically acceptable salt thereof having Formula (Ic) or (Id):

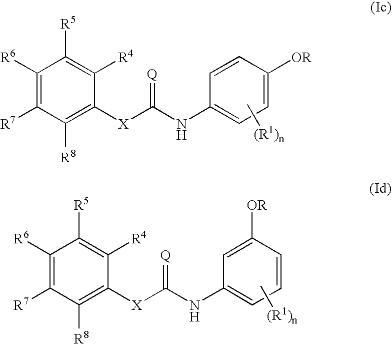

wherein Q, X, n, R¹, R⁴, R⁵, R⁶, R⁷, R⁸, and R are previously defined.

Various embodiments relating to the compounds or pharmaceutically acceptable salts of Formula (Ic) and (Id) are listed below. These embodiments can be combined with each other or with any other embodiments described in this application. In some embodiments, provided are compounds of Formula (Ic) or (Id) having one or more of the following features.

In some embodiments of Formula (Ic) or (Id) X is NH. In some embodiments of Formula (Ic) or (Id) X is CH₂. In some embodiments of Formula (Ic) or (Id) X is a covalent bond.

In some embodiments Q is O.

In some embodiments R is substituted alkyl. In some embodiments, R is alkyl substituted with aryl, heterocycloalkyl, or substituted heterocycloalkyl. In some embodiments, R is benzyl. In some embodiments, R is alkyl substituted with NR²R³ wherein R² and R³ together form a morpholino or piperazinyl ring, wherein said ring may be substituted or unsubstituted.

In some embodiments R is phenyl or substituted phenyl. In some embodiments, the R is phenyl substituted with —C(O)OH.

In some embodiments n is 0.

In some embodiments n is 1 and R¹ is halo. In some embodiments, R¹ is fluoro.

In some embodiments R⁴ and R⁸ are hydrogen.

In some embodiments at least one of R⁴ and R⁸ is fluoro or chloro. In some embodiments one of R⁴ and R⁸ is fluoro, and the other of R⁴ and R⁸ is hydrogen. In some embodiments, both R⁴ and R⁸ are fluoro or chloro.

In some embodiments R⁵, R⁶, and R⁷ are independently selected from the group consisting of hydrogen, halo, alkyl, haloalkyl, haloalkoxy, alkylamino, heterocycloalkyloxy, alkylthio, haloalkylthio, cyano, alkylsulfonyl, and haloalkylsulfonyl.

In some embodiments at least one of R⁵, R⁶, and R⁷ is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl.

In some embodiments one of R⁵, R⁶, and R⁷ is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl, and the remainder of R⁵, R⁶, and R⁷ are hydrogen.

In some embodiments at least one of R⁵, R⁶, and R⁷ is selected from the group consisting of halo, trifluoromethyl, trifluoromethoxy, alkylsulfonyl, and haloalkylsulfonyl.

In some embodiments R⁶ is selected from the group consisting of chloro, fluoro, trifluoromethyl, and trifluoromethoxy. In some embodiments, R⁴, R⁵, R⁷, and R⁸ are hydrogen.

In some embodiments R⁵ is selected from the group consisting of chloro, fluoro, trifluoromethyl, and trifluoromethoxy and R⁴, R⁶, R⁷, and R⁸ are hydrogen.

In some embodiments, R⁶ and R⁷ together form a heterocycloalkyl ring. In some embodiments, Y is

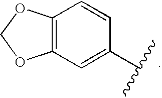

In other embodiments, provided is a compound, stereoisomer, or pharmaceutically acceptable salt thereof having Formula (Ie) or (If):

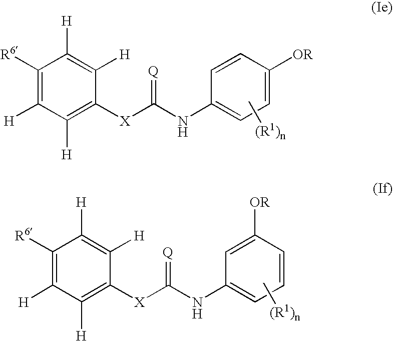

wherein Q, X, n, R and R¹ are previously defined and R^{6′} is selected from the group consisting of halo, haloalkyl, haloalkoxy, alkylthio, haloalkylthio, cyano, alkylsulfonyl, and haloalkylsulfonyl.

Various embodiments relating to the compounds or pharmaceutically acceptable salts of Formula (Ie) and (If) are listed below. These embodiments can be combined with each other or with any other embodiments described in this application. In some aspects, provided are compounds of Formula (Ie) or (If) having one or more of the following features.

In some embodiments of Formula (Ie) or (If), X is NH. In some embodiments X is CH₂. In some embodiments X is a covalent bond.

In some embodiments Q is O.

In some embodiments R is substituted alkyl. In some embodiments, R is alkyl substituted with aryl, heterocycloalkyl, or substituted heterocycloalkyl. In some embodiments, R is alkyl substituted with NR²R³ wherein R² and R³ together form a morpholino or piperazinyl ring, wherein said ring may be substituted or unsubstituted.

In some embodiments R is phenyl or substituted phenyl. In some embodiments, R is phenyl substituted with —C(O)OH.

In some embodiments n is 0.

In some embodiments n is 1 and R¹ is halo. In some embodiments, R¹ is fluoro.

In some embodiments R⁶ is selected from the group consisting of halo, trifluoromethyl, trifluoromethoxy, alkylsulfonyl, and haloalkylsulfonyl. In some embodiments, R^{6′} is selected from the group consisting of chloro, fluoro, and trifluoromethyl. In some embodiments, R^{6′} is trifluoromethoxy.

In some embodiments provided is a compound or a pharmaceutically acceptable salt thereof selected from Table 1.

TABLE 1

| Compound | Structure | Name |
|---|---|---|
| 1 | 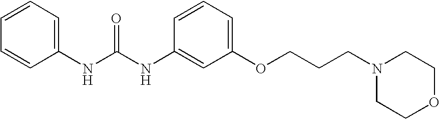 | 1-[3-(3-Morpholin-4-yl-propoxy)-phenyl]-3-phenyl-urea |
| 2 | 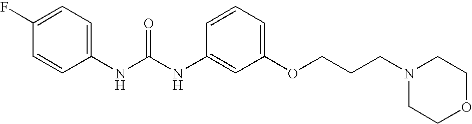 | 1-(4-Fluoro-phenyl)-3-[3-(3-morpholin-4-yl-propoxy)-phenyl]-urea |
| 3 | 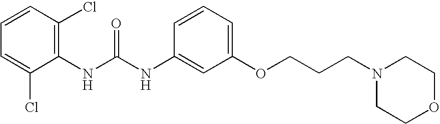 | 1-(2,6-Dichloro-phenyl)-3-[3-(3-morpholin-4-yl-propoxy)-phenyl]-urea |
| 4 | 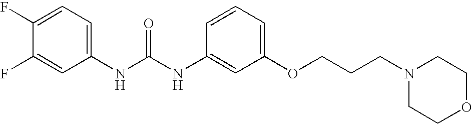 | 1-(3,4-Difluoro-phenyl)-3-[3-(3-morpholin-4-yl-propoxy)-phenyl]-urea |
| 5 | 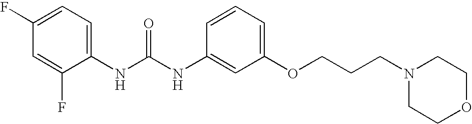 | 1-(2,4-Difluoro-phenyl)-3-[3-(3-morpholin-4-yl-propoxy)-phenyl]-urea |
| 6 | 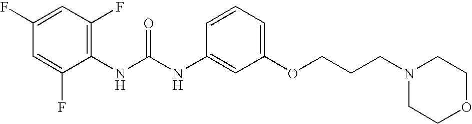 | 1-[3-(3-Morpholin-4-yl-propoxy)-phenyl]-3-(2,4,6-trifluoro-phenyl)-urea |
| 7 | 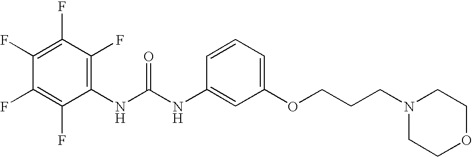 | 1-[3-(3-Morpholin-4-yl-propoxy)-phenyl]-3-pentafluorophenyl-urea |
| 8 | 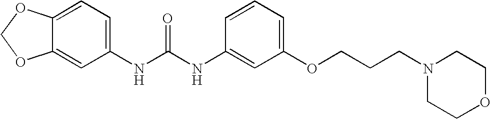 | 1-Benzo[1,3]dioxol-5-yl-3-[3-(3-morpholin-4-yl-propoxy)-phenyl]-urea |
| 9 | 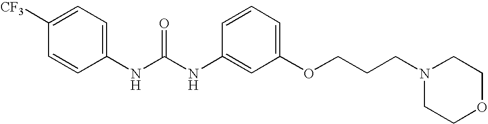 | 1-[3-(3-Morpholin-4-yl-propoxy)-phenyl]-3-(4-trifluoromethyl-phenyl)-urea |
| 10 | 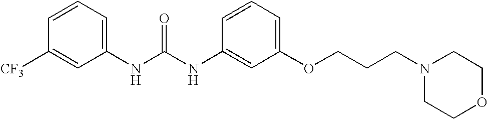 | 1-[3-(3-Morpholin-4-yl-propoxy)-phenyl]-3-(3-trifluoromethyl-phenyl)-urea |
| 11 | 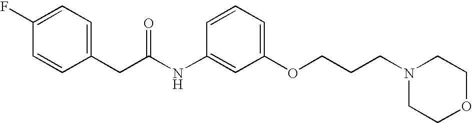 | 2-(4-Fluoro-phenyl)-N-[3-(3-morpholin-4-yl-propoxy)-phenyl]-acetamide |
| 12 | 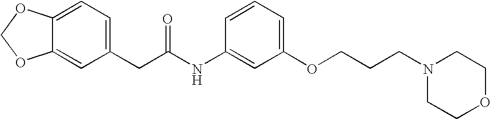 | 2-Benzo[1,3]dioxol-5-yl-N-[3-(3-morpholin-4-yl-propoxy)-phenyl]-acetamide |
| 13 | 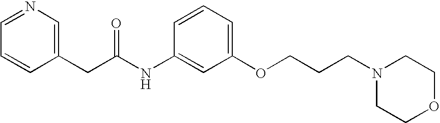 | N-[3-(3-Morpholin-4-yl-propoxy)-phenyl]-2-pyridin-3-yl-acetamide |
| 14 | 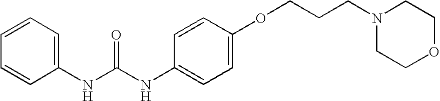 | 1-[4-(3-Morpholin-4-yl-propoxy)-phenyl]-3-phenyl-urea |
| 15 | 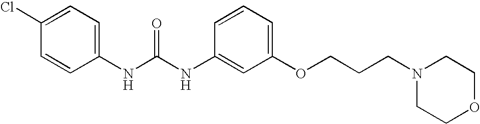 | 1-(4-Chloro-phenyl)-3-[3-(3-morpholin-4-yl-propoxy)-phenyl]-urea |
| 16 | 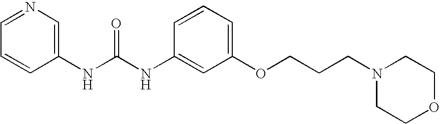 | 1-[3-(3-Morpholin-4-yl-propoxy)-phenyl]-3-pyridin-3-yl-urea |
| 17 | 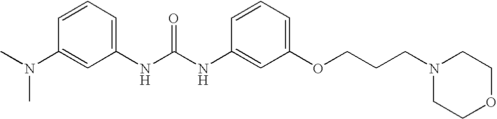 | 1-(3-Dimethylamino-phenyl)-3-[3-(3-morpholin-4-yl-propoxy)-phenyl]-urea |
| 18 | 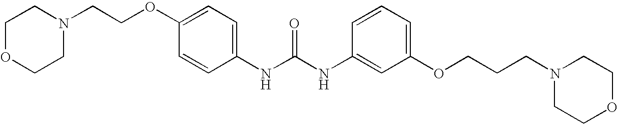 | 1-[4-(2-Morpholin-4-yl-ethoxy)-phenyl]-3-[3-(3-morpholin-4-yl-propoxy)-phenyl]-urea |
| 19 | 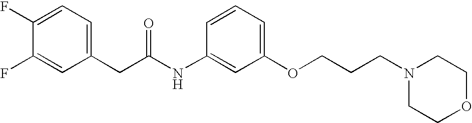 | 2-(3,4-Difluoro-phenyl)-N-[3-(3-morpholin-4-yl-propoxy)-phenyl]-acetamide |
| 20 | 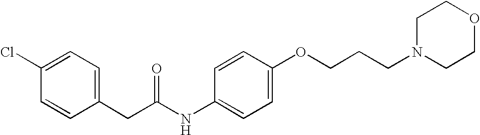 | 2-(4-Chloro-phenyl)-N-[4-(3-morpholin-4-yl-propoxy)-phenyl]-acetamide |
| 21 | 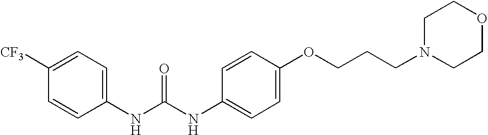 | 1-[4-(3-Morpholin-4-yl-propoxy)-phenyl]-3-(4-trifluoromethyl-phenyl)-urea |
| 22 | 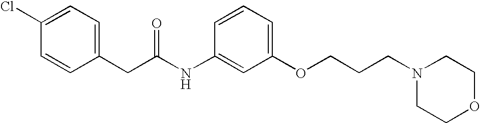 | 2-(4-Chloro-phenyl)-N-[3-(3-morpholin-4-yl-propoxy)-phenyl]-acetamide |
| 23 | 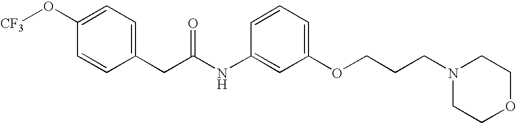 | N-[3-(3-Morpholin-4-yl-propoxy)-phenyl]-2-(4-trifluoromethoxy-phenyl)-acetamide |
| 24 | 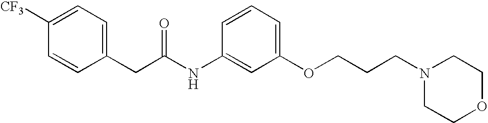 | N-[3-(3-Morpholin-4-yl-propoxy)-phenyl]-2-(4-trifluoromethyl-phenyl)-acetamide |
| 25 | 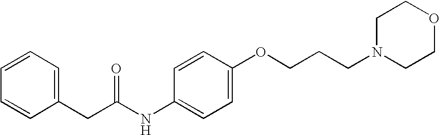 | N-[4-(3-Morpholin-4-yl-propoxy)-phenyl]-2-phenyl-acetamide |
| 26 | 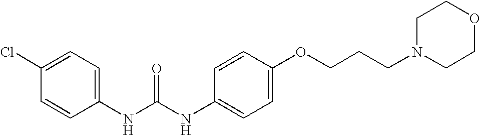 | 1-(4-Chloro-phenyl)-3-[4-(3-morpholin-4-yl-propoxy)-phenyl]-urea |
| 27 | 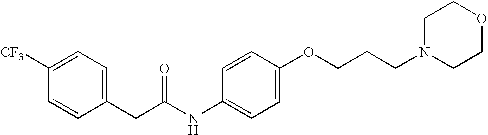 | N-[4-(3-Morpholin-4-yl-propoxy)-phenyl]-2-(4-trifluoromethyl-phenyl)-acetamide |
| 28 | 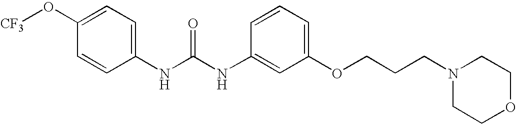 | 1-[3-(3-Morpholin-4-yl-propoxy)-phenyl]-3-(4-trifluoromethoxy-phenyl)-urea |
| 29 | 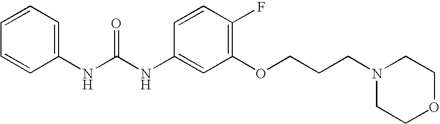 | 1-[4-Fluoro-3-(3-morpholin-4-yl-propoxy)-phenyl]-3-phenyl-urea |
| 30 | 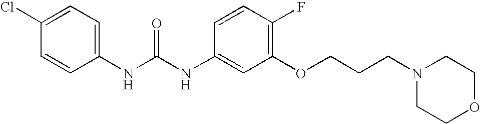 | 1-(4-Chloro-phenyl)-3-[4-fluoro-3-(3-morpholin-4-yl-propoxy)-phenyl]-urea |
| 31 | 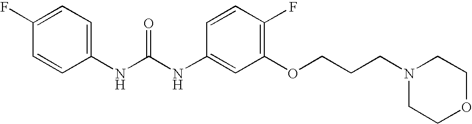 | 1-[4-Fluoro-3-(3-morpholin-4-yl-propoxy)-phenyl]-3-(4-fluoro-phenyl)-urea |
| 32 | 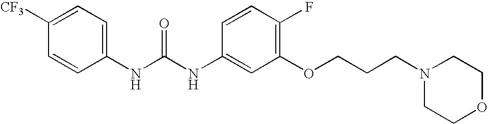 | 1-[4-Fluoro-3-(3-morpholin-4-yl-propoxy)-phenyl]-3-(4-trifluoromethyl-phenyl)-urea |
| 33 | 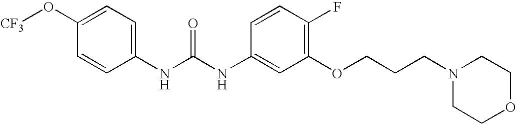 | 1-[4-Fluoro-3-(3-morpholin-4-yl-propoxy)-phenyl]-3-(4-trifluoromethoxy-phenyl)-urea |
| 34 | 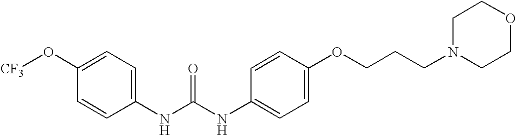 | 1-[4-(3-Morpholin-4-yl-propoxy)-phenyl]-3-(4-trifluoromethoxy-phenyl)-urea |
| 35 | 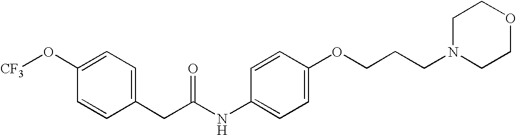 | N-[4-(3-Morpholin-4-yl-propoxy)-phenyl]-2-(4-trifluoromethoxy-phenyl)-acetamide |
| 36 | 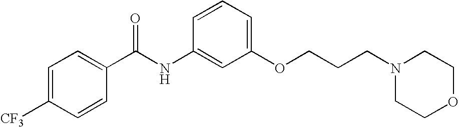 | N-[3-(3-Morpholin-4-yl-propoxy)-phenyl]-4-trifluoromethyl-benzamide |
| 37 | 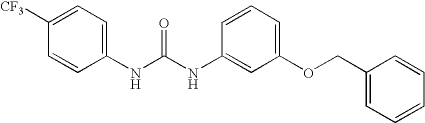 | 1-(3-Benzyloxy-phenyl)-3-(4-trifluoromethyl-phenyl)-urea |
| 38 | 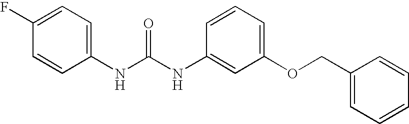 | 1-(3-Benzyloxy-phenyl)-3-(4-fluoro-phenyl)-urea |
| 39 | 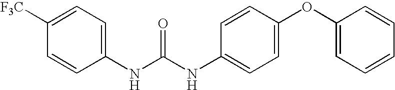 | 1-(4-Phenoxy-phenyl)-3-(4-trifluoromethyl-phenyl)-urea |
| 40 | 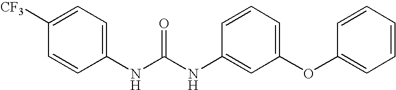 | 1-(3-Phenoxy-phenyl)-3-(4-trifluoromethyl-phenyl)-urea |
| 60 | 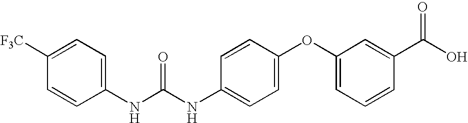 | 3-(4-(3-(4-(trifluoromethyl)phenyl)ureido)phenoxy)benzoic acid |
| 64 | 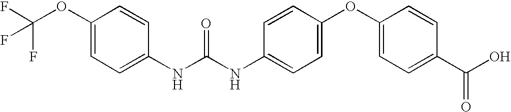 | 4-(4-(3-(4-(trifluoromethoxy)phenyl)ureido)phenoxy)benzoic acid |
| 66 | 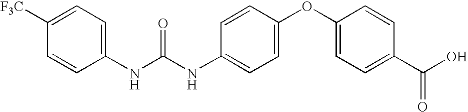 | 4-(4-(3-(4-(trifluoromethyl)phenyl)ureido)phenoxy)benzoicacid |
| 67 | 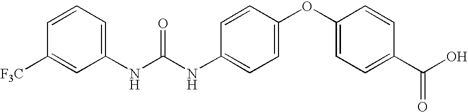 | 4-(4-(3-(3-(trifluoromethyl)phenyl)ureido)phenoxy)benzoic acid |
| 69 | 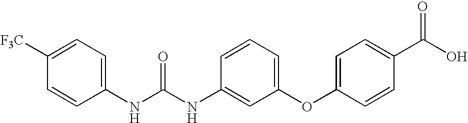 | 4-(3-(3-(4-(trifluoromethyl)phenyl)ureido)phenoxy)benzoicacid |
| 71 | 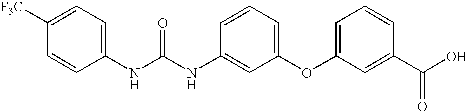 | 3-(3-(3-(4-(trifluoromethyl)phenyl)ureido)phenoxy)benzoicacid |

In one embodiment provided is a compound or a pharmaceutically acceptable salt thereof selected from Table 2.

TABLE 2

| Compound | Structure | Name |
|---|---|---|
| 41 | 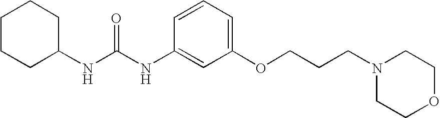 | 1-Cyclohexyl-3-[3-(3-morpholin-4-yl-propoxy)-phenyl]-urea |
| 42 | 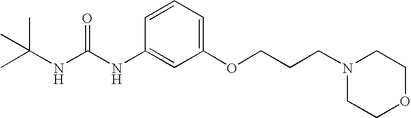 | 1-tert-Butyl-3-[3-(3-morpholin-4-yl-propoxy)-phenyl]-urea |
| 43 | 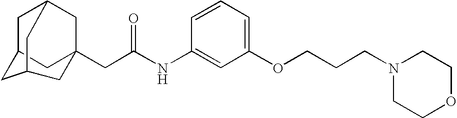 | 2-Adamantan-1-yl-N-[3-(3-morpholin-4-yl-propoxy)-phenyl]-acetamide |
| 44 | 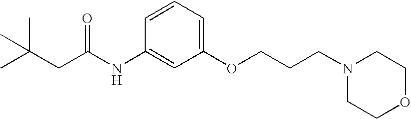 | 3,3-Dimethyl-N-[3-(3-morpholin-4-yl-propoxy)-phenyl]-butyramide |
| 45 | 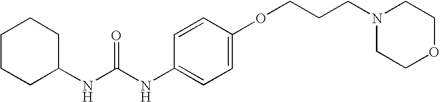 | 1-Cyclohexyl-3-[4-(3-morpholin-4-yl-propoxy)-phenyl]-urea |
| 46 | 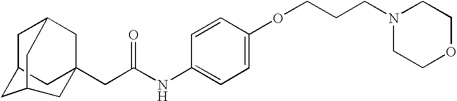 | 2-Adamantan-1-yl-N-[4-(3-morpholin-4-yl-propoxy)-phenyl]-acetamide |
| 47 | 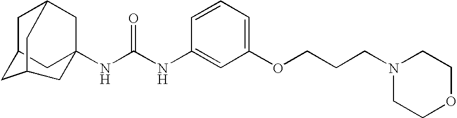 | 1-Adamantan-1-yl-3-[3-(3-morpholin-4-yl-propoxy)-phenyl]-urea |
| 48 | 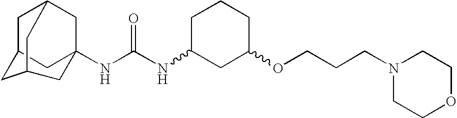 | 1-Adamantan-1-yl-3-[3-(3-morpholin-4-yl-propoxy)-cyclohexyl]-urea |
| 49 | 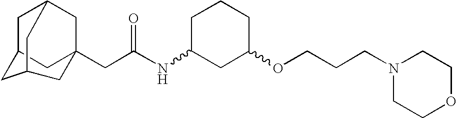 | 2-Adamantan-1-yl-N-[3-(3-morpholin-4-yl-propoxy)-cyclohexyl]-acetamide |
| 50 | 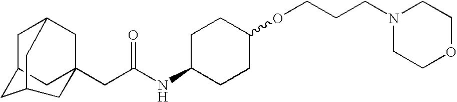 | 2-Adamantan-1-yl-N-[4-(3-morpholin-4-yl-propoxy)-cyclohexyl]-acetamide |
| 51 | 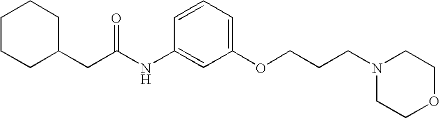 | 2-Cyclohexyl-N-[3-(3-morpholin-4-yl-propoxy)-phenyl]-acetamide |
| 52 | 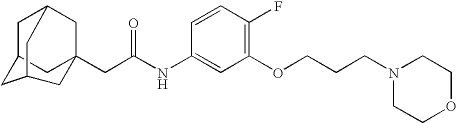 | 2-Adamantan-1-yl-N-[4-fluoro-3-(3-morpholin-4-yl-propoxy)-phenyl]-acetamide |
| 53 | 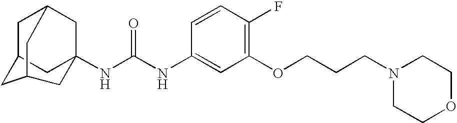 | 1-Adamantan-1-yl-3-[4-fluoro-3-(3-morpholin-4-yl-propoxy)-phenyl]-urea |
| 54 | 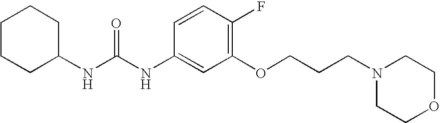 | 1-Cyclohexyl-3-[4-fluoro-3-(3-morpholin-4-yl-propoxy)-phenyl]-urea |
| 55 | 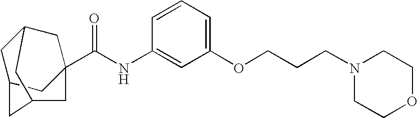 | Adamantane-1-carboxylicacid [3-(3-morpholin-4-yl-propoxy)-phenyl]-amide |
| 56 | 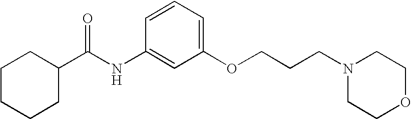 | Cyclohexanecarboxylicacid [3-(3-morpholin-4-yl-propoxy)-phenyl]-amide |
| 57 | 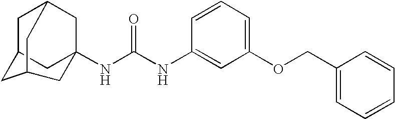 | 1-Adamantan-1-yl-3-(3-benzyloxy-phenyl)-urea |
| 58 | 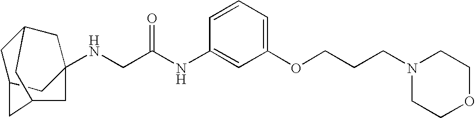 | 2-(Adamantan-1-ylamino)-N-[3-(3-morpholin-4-yl-propoxy)-phenyl]-acetamide |
| 59 | 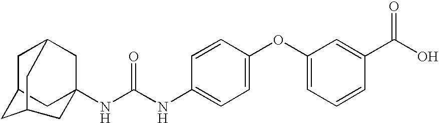 | 3-(4-(3-Adamantan-1-ylureido)phenoxy)benzoicacid |
| 61 | 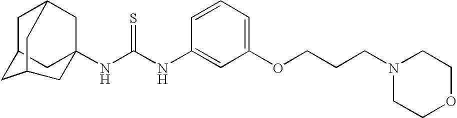 | 1-adamantan-1-yl-3-[3-(3-morpholin-4-yl-propoxy)cyclohexyl]thiourea |
| 62 | 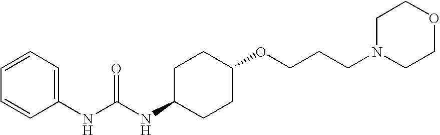 | 1-[4-(3-morpholin-4-yl-propoxy)cyclohexyl]-3-phenylurea |
| 63 | 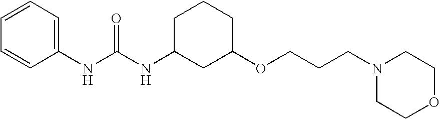 | 1-[3-(3-Morpholin-4-yl-propoxy)cyclohexyl]-3-phenylurea |
| 65 | 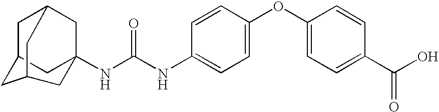 | 4-(4-(3-(adamantyl)ureido)phenoxy)benzoic acid |
| 68 | 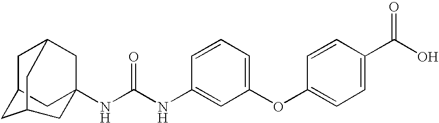 | 4-(3-(3-(adamantyl)ureido)phenoxy)benzoicacid |
| 70 | 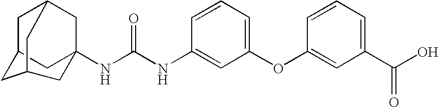 | 3-(3-(3-(adamantyl)ureido)phenoxy)benzoicacid |
| 72 | 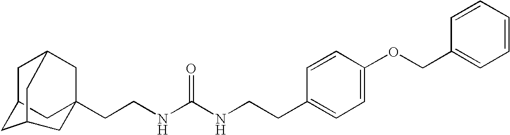 | 1-(4-(benzyloxy)phenethyl)-3-(adamantylethyl)urea |
| 73 | 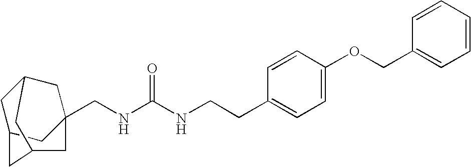 | 1-(4-(benzyloxy)phenethyl)-3-(adamantylmethyl)urea |
| 74 | 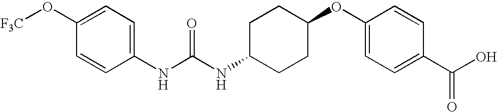 | 4-((1r,4r)-4-(3-(4-(trifluoromethoxy)phenyl)ureido)cyclohexyloxy)benzoic acid |
| 75 | 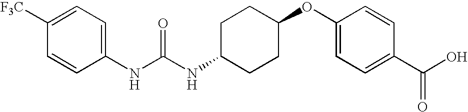 | 4-((1R,4R)-4-(3-(4-(trifluoromethyl)phenyl)ureido)cyclohexyl)benzoic acid |

In another aspect of this invention, provided is a pharmaceutical composition comprising a pharmaceutically acceptable carrier and a therapeutically effective amount of a compound or pharmaceutically acceptable salt of any one of Formula (I′), (I), (Ia)-(If) or of Tables 1 or 2 for treating a soluble expoxide hydrolase mediated disease.

In another aspect of the invention, provided is a method for treating a soluble expoxide hydrolase mediated disease, said method comprising administering to a patient a pharmaceutical composition comprising a pharmaceutically acceptable carrier and a therapeutically effective amount of a compound or pharmaceutically acceptable salt of Formula (II):

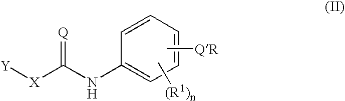

wherein:
Q is O or S;
Q′ is O or S;
R is selected from the group consisting of alkyl, substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3;
X is selected from the group consisting of a covalent bond, NH, or CR′R″ where R′ and R″ are independently H or alkyl or R′ and R″ together form a C₃-C₆ cycloalkyl ring; and
Y is selected from the group consisting of heteroaryl, substituted heteroaryl, and

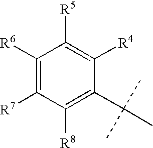

wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring.

In some embodiments, said method comprising administering to a patient a pharmaceutical composition comprising a pharmaceutically acceptable carrier and a therapeutically effective amount of a compound or pharmaceutically acceptable salt of any one of Formula (I′), (I), (Ia)-(If) or of Tables 1 or 2.

It has previously been shown that inhibitors of soluble epoxide hydrolase (“sEH”) can reduce hypertension (see, e.g., U.S. Pat. No. 6,351,506). Such inhibitors can be useful in controlling the blood pressure of persons with undesirably high blood pressure, including those who suffer from diabetes.

In preferred embodiments, compounds of the invention are administered to a subject in need of treatment for hypertension, specifically renal, hepatic, or pulmonary hypertension; inflammation, specifically renal inflammation, hepatic inflammation, vascular inflammation, and lung inflammation; adult respiratory distress syndrome; diabetic complications; end stage renal disease; Raynaud syndrome; and arthritis.

### Methods to Treat ARDS and SIRS

Adult respiratory distress syndrome (ARDS) is a pulmonary disease that has a mortality rate of 50% and results from lung lesions that are caused by a variety of conditions found in trauma patients and in severe burn victims. Ingram, R. H. Jr., “Adult Respiratory Distress Syndrome,” Harrison's Principals of Internal Medicine, 13, p. 1240, 1995. With the possible exception of glucocorticoids, there have not been therapeutic agents known to be effective in preventing or ameliorating the tissue injury, such as microvascular damage, associated with acute inflammation that occurs during the early development of ARDS.

ARDS, which is defined in part by the development of alveolar edema, represents a clinical manifestation of pulmonary disease resulting from both direct and indirect lung injury. While previous studies have detailed a seemingly unrelated variety of causative agents, the initial events underlying the pathophysiology of ARDS are not well understood. ARDS was originally viewed as a single organ failure, but is now considered a component of the multisystem organ failure syndrome (MOFS). Pharmacologic intervention or prevention of the inflammatory response is presently viewed as a more promising method of controlling the disease process than improved ventilatory support techniques. See, for example, Demling, Annu. Rev. Med., 46, pp. 193-203, 1995.

Another disease (or group of diseases) involving acute inflammation is the systematic inflammatory response syndrome, or SIRS, which is the designation recently established by a group of researchers to describe related conditions resulting from, for example, sepsis, pancreatitis, multiple trauma such as injury to the brain, and tissue injury, such as laceration of the musculature, brain surgery, hemorrhagic shock, and immune-mediated organ injuries (JAMA, 268(24):3452-3455 (1992)).

The ARDS ailments are seen in a variety of patients with severe burns or sepsis. Sepsis in turn is one of the SIRS symptoms. In ARDS, there is an acute inflammatory reaction with high numbers of neutrophils that migrate into the interstitium and alveoli. If this progresses there is increased inflammation, edema, cell proliferation, and the end result is impaired ability to extract oxygen. ARDS is thus a common complication in a wide variety of diseases and trauma. The only treatment is supportive. There are an estimated 150,000 cases per year and mortality ranges from 10% to 90%.

The exact cause of ARDS is not known. However it has been hypothesized that over-activation of neutrophils leads to the release of linoleic acid in high levels via phospholipase A₂ activity. Linoleic acid in turn is converted to 9,10-epoxy-12-octadecenoate enzymatically by neutrophil cytochrome P-450 epoxygenase and/or a burst of active oxygen. This lipid epoxide, or leukotoxin, is found in high levels in burned skin and in the serum and bronchial lavage of burn patients. Furthermore, when injected into rats, mice, dogs, and other mammals it causes ARDS. The mechanism of action is not known. However, the leukotoxin diol produced by the action of the soluble epoxide hydrolase appears to be a specific inducer of the mitochondrial inner membrane permeability transition (MPT). This induction by leukotoxin diol, the diagnostic release of cytochrome c, nuclear condensation, DNA laddering, and CPP32 activation leading to cell death were all inhibited by cyclosporin A, which is diagnostic for MPT induced cell death. Actions at the mitochondrial and cell level were consistent with this mechanism of action suggesting that the inhibitors of this invention could be used therapeutically with compounds which block MPT.

Thus in one embodiment provided is a method for treating ARDS. In another embodiment, provided is a method for treating SIRS.

### Methods for Inhibiting Progression of Kidney Deterioration (Nephropathy) and Reducing Blood Pressure:

In another aspect of the invention, the compounds of the invention can reduce damage to the kidney, and especially damage to kidneys from diabetes, as measured by albuminuria. The compounds of the invention can reduce kidney deterioration (nephropathy) from diabetes even in individuals who do not have high blood pressure. The conditions of therapeutic administration are as described above.

cis-Epoxyeicosantrienoic acids (“EETs”) can be used in conjunction with the compounds of the invention to further reduce kidney damage. EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into DHETs is reduced. Without wishing to be bound by theory, it is believed that raising the level of EETs interferes with damage to kidney cells by the microvasculature changes and other pathologic effects of diabetic hyperglycemia. Therefore, raising the EET level in the kidney is believed to protect the kidney from progression from microalbuminuria to end stage renal disease.

EETs are well known in the art. EETs useful in the methods of the present invention include 14,15-EET, 8,9-EET and 11,12-EET, and 5,6 EETs, in that order of preference. Preferably, the EETs are administered as the methyl ester, which is more stable. Persons of skill will recognize that the EETs are regioisomers, such as 8S,9R— and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

EETs produced by the endothelium have anti-hypertensive properties and the EETs 11,12-EET and 14,15-EET may be endothelium-derived hyperpolarizing factors (EDHFs). Additionally, EETs such as 11,12-EET have profibrinolytic effects, anti-inflammatory actions and inhibit smooth muscle cell proliferation and migration. In the context of the present invention, these favorable properties are believed to protect the vasculature and organs during renal and cardiovascular disease states.

Inhibition of sEH activity can be effected by increasing the levels of EETs. This permits EETs to be used in conjunction with one or more sEH inhibitors to reduce nephropathy in the methods of the invention. It further permits EETs to be used in conjunction with one or more sEH inhibitors to reduce hypertension, or inflammation, or both. Thus, medicaments of EETs can be made which can be administered in conjunction with one or more sEH inhibitors, or a medicament containing one or more sEH inhibitors can optionally contain one or more EETs.

The EETs can be administered concurrently with the sEH inhibitor, or following administration of the sEH inhibitor. It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, therefore, it is desirable that the EETs be administered during the period in which the inhibitor will be present in amounts to be effective to delay hydrolysis of the EETs. Typically, the EET or EETs will be administered within 48 hours of administering an sEH inhibitor. Preferably, the EET or EETs are administered within 24 hours of the inhibitor, and even more preferably within 12 hours. In increasing order of desirability, the EET or EETs are administered within 10, 8, 6, 4, 2, hours, 1 hour, or one half hour after administration of the inhibitor. Most preferably, the EET or EETs are administered concurrently with the inhibitor.

In preferred embodiments, the EETs, the compound of the invention, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage through the acidic environment of the stomach into the basic environment of the intestines. Such coatings are well known in the art. For example, aspirin coated with so-called “enteric coatings” is widely available commercially. Such enteric coatings may be used to protect EETs during passage through the stomach. An exemplary coating is set forth in the Examples.

While the anti-hypertensive effects of EETs have been recognized, EETs have not been administered to treat hypertension because it was thought endogenous sEH would hydrolyse the EETs too quickly for them to have any useful effect. Surprisingly, it was found during the course of the studies underlying the present invention that exogenously administered inhibitors of sEH succeeded in inhibiting sEH sufficiently that levels of EETs could be further raised by the administration of exogenous EETs. These findings underlie the co-administration of sEH inhibitors and of EETs described above with respect to inhibiting the development and progression of nephropathy. This is an important improvement in augmenting treatment. While levels of endogenous EETs are expected to rise with the inhibition of sEH activity caused by the action of the sEH inhibitor, and therefore to result in at least some improvement in symptoms or pathology, it may not be sufficient in all cases to inhibit progression of kidney damage fully or to the extent intended. This is particularly true where the diseases or other factors have reduced the endogenous concentrations of EETs below those normally present in healthy individuals. Administration of exogenous EETs in conjunction with an sEH inhibitor is therefore expected to be beneficial and to augment the effects of the sEH inhibitor in reducing the progression of diabetic nephropathy.

The present invention can be used with regard to any and all forms of diabetes to the extent that they are associated with progressive damage to the kidney or kidney function. The chronic hyperglycemia of diabetes is associated with long-term damage, dysfunction, and failure of various organs, especially the eyes, kidneys, nerves, heart, and blood vessels. The long-term complications of diabetes include retinopathy with potential loss of vision; nephropathy leading to renal failure; peripheral neuropathy with risk of foot ulcers, amputation, and Charcot joints.

In addition, persons with metabolic syndrome are at high risk of progression to type 2 diabetes, and therefore at higher risk than average for diabetic nephropathy. It is therefore desirable to monitor such individuals for microalbuminuria, and to administer an sEH inhibitor and, optionally, one or more EETs, as an intervention to reduce the development of nephropathy. The practitioner may wait until microalbuminuria is seen before beginning the intervention. Since a person can be diagnosed with metabolic syndrome without having a blood pressure of 130/85 or higher, both persons with blood pressure of 130/85 or higher and persons with blood pressure below 130/85 can benefit from the administration of sEH inhibitors and, optionally, of one or more EETs, to slow the progression of damage to their kidneys. In some preferred embodiments, the person has metabolic syndrome and blood pressure below 130/85.

Dyslipidemia or disorders of lipid metabolism is another risk factor for heart disease. Such disorders include an increased level of LDL cholesterol, a reduced level of HDL cholesterol, and an increased level of triglycerides. An increased level of serum cholesterol, and especially of LDL cholesterol, is associated with an increased risk of heart disease. The kidneys are also damaged by such high levels. It is believed that high levels of triglycerides are associated with kidney damage. In particular, levels of cholesterol over 200 mg/dL, and especially levels over 225 mg/dL, would suggest that sEH inhibitors and, optionally, EETs, should be administered. Similarly, triglyceride levels of more than 215 mg/dL, and especially of 250 mg/dL or higher, would indicate that administration of sEH inhibitors and, optionally, of EETs, would be desirable. The administration of compounds of the present invention with or without the EETs, can reduce the need to administer statin drugs (HMG-COA reductase inhibitors) to the patients, or reduce the amount of the statins needed. In some embodiments, candidates for the methods, uses, and compositions of the invention have triglyceride levels over 215 mg/dL and blood pressure below 130/85. In some embodiments, the candidates have triglyceride levels over 250 mg/dL and blood pressure below 130/85. In some embodiments, candidates for the methods, uses and compositions of the invention have cholesterol levels over 200 mg/dL and blood pressure below 130/85. In some embodiments, the candidates have cholesterol levels over 225 mg/dL and blood pressure below 130/85.

### Methods of Inhibiting the Proliferation of Vascular Smooth Muscle Cells:

In other embodiments, compounds of Formula (I′), (I), (Ia)-(If), (II), or of Tables 1 or 2 inhibit proliferation of vascular smooth muscle (VSM) cells without significant cell toxicity, (e.g. specific to VSM cells). Because VSM cell proliferation is an integral process in the pathophysiology of atherosclerosis, these compounds are suitable for slowing or inhibiting atherosclerosis. These compounds are useful to subjects at risk for atherosclerosis, such as individuals who have diabetes and those who have had a heart attack or a test result showing decreased blood circulation to the heart. The conditions of therapeutic administration are as described above.

The methods of the invention are particularly useful for patients who have had percutaneous intervention, such as angioplasty to reopen a narrowed artery, to reduce or to slow the narrowing of the reopened passage by restenosis. In some preferred embodiments, the artery is a coronary artery. The compounds of the invention can be placed on stents in polymeric coatings to provide a controlled localized release to reduce restenosis. Polymer compositions for implantable medical devices, such as stents, and methods for embedding agents in the polymer for controlled release, are known in the art and taught, for example, in U.S. Pat. Nos. 6,335,029; 6,322,847; 6,299,604; 6,290,722; 6,287,285; and 5,637,113. In preferred embodiments, the coating releases the inhibitor over a period of time, preferably over a period of days, weeks, or months. The particular polymer or other coating chosen is not a critical part of the present invention.

The methods of the invention are useful for slowing or inhibiting the stenosis or restenosis of natural and synthetic vascular grafts. As noted above in connection with stents, desirably, the synthetic vascular graft comprises a material which releases a compound of the invention over time to slow or inhibit VSM proliferation and the consequent stenosis of the graft. Hemodialysis grafts are a particularly preferred embodiment.

In addition to these uses, the methods of the invention can be used to slow or to inhibit stenosis or restenosis of blood vessels of persons who have had a heart attack, or whose test results indicate that they are at risk of a heart attack.

Removal of a clot such as by angioplasty or treatment with tissue plasminogen activator (tPA) can also lead to reperfusion injury, in which the resupply of blood and oxygen to hypoxic cells causes oxidative damage and triggers inflammatory events. In some embodiments, provided are methods for administering the compounds and compositions of the invention for treating reperfusion injury. In some such embodiments, the compounds and compositions are administered prior to or following angioplasty or administration of tPA.

In one group of preferred embodiments, compounds of the invention are administered to reduce proliferation of VSM cells in persons who do not have hypertension. In another group of embodiments, compounds of the invention are used to reduce proliferation of VSM cells in persons who are being treated for hypertension, but with an agent that is not an sEH inhibitor.

The compounds of the invention can be used to interfere with the proliferation of cells which exhibit inappropriate cell cycle regulation. In one important set of embodiments, the cells are cells of a cancer. The proliferation of such cells can be slowed or inhibited by contacting the cells with a compound of the invention. The determination of whether a particular compound of the invention can slow or inhibit the proliferation of cells of any particular type of cancer can be determined using assays routine in the art.

In addition to the use of the compounds of the invention, the levels of EETs can be raised by adding EETs. VSM cells contacted with both an EET and a compound of the invention exhibited slower proliferation than cells exposed to either the EET alone or to the compound of the invention alone. Accordingly, if desired, the slowing or inhibition of VSM cells of a compound of the invention can be enhanced by adding an EET along with a compound of the invention. In the case of stents or vascular grafts, for example, this can conveniently be accomplished by embedding the EET in a coating along with a compound of the invention so that both are released once the stent or graft is in position.

### Methods of Inhibiting the Progression of Obstructive Pulmonary Disease, Interstitial Lung Disease, or Asthma:

Chronic obstructive pulmonary disease, or COPD, encompasses two conditions, emphysema and chronic bronchitis, which relate to damage caused to the lung by air pollution, chronic exposure to chemicals, and tobacco smoke. Emphysema as a disease relates to damage to the alveoli of the lung, which results in loss of the separation between alveoli and a consequent reduction in the overall surface area available for gas exchange. Chronic bronchitis relates to irritation of the bronchioles, resulting in excess production of mucin, and the consequent blocking by mucin of the airways leading to the alveoli. While persons with emphysema do not necessarily have chronic bronchitis or vice versa, it is common for persons with one of the conditions to also have the other, as well as other lung disorders.

Some of the damage to the lungs due to COPD, emphysema, chronic bronchitis, and other obstructive lung disorders can be inhibited or reversed by administering inhibitors of the enzyme known as soluble epoxide hydrolase, or “sEH”. The effects of sEH inhibitors can be increased by also administering EETs. The effect is at least additive over administering the two agents separately, and may indeed be synergistic.

The studies reported herein show that EETs can be used in conjunction with sEH inhibitors to reduce damage to the lungs by tobacco smoke or, by extension, by occupational or environmental irritants. These findings indicate that the co-administration of sEH inhibitors and of EETs can be used to inhibit or slow the development or progression of COPD, emphysema, chronic bronchitis, or other chronic obstructive lung diseases which cause irritation to the lungs.

Animal models of COPD and humans with COPD have elevated levels of immunomodulatory lymphocytes and neutrophils. Neutrophils release agents that cause tissue damage and, if not regulated, will over time have a destructive effect. Without wishing to be bound by theory, it is believed that reducing levels of neutrophils reduces tissue damage contributing to obstructive lung diseases such as COPD, emphysema, and chronic bronchitis. Administration of sEH inhibitors to rats in an animal model of COPD resulted in a reduction in the number of neutrophils found in the lungs. Administration of EETs in addition to the sEH inhibitors also reduced neutrophil levels. The reduction in neutrophil levels in the presence of sEH inhibitor and EETs was greater than in the presence of the sEH inhibitor alone.

While levels of endogenous EETs are expected to rise with the inhibition of sEH activity caused by the action of the sEH inhibitor, and therefore to result in at least some improvement in symptoms or pathology, it may not be sufficient in all cases to inhibit progression of COPD or other pulmonary diseases. This is particularly true where the diseases or other factors have reduced the endogenous concentrations of EETs below those normally present in healthy individuals. Administration of exogenous EETs in conjunction with an sEH inhibitor is therefore expected to augment the effects of the sEH inhibitor in inhibiting or reducing the progression of COPD or other pulmonary diseases.

In addition to inhibiting or reducing the progression of chronic obstructive airway conditions, the invention also provides new ways of reducing the severity or progression of chronic restrictive airway diseases. While obstructive airway diseases tend to result from the destruction of the lung parenchyma, and especially of the alveoli, restrictive diseases tend to arise from the deposition of excess collagen in the parenchyma. These restrictive diseases are commonly referred to as “interstitial lung diseases”, or “ILDs”, and include conditions such as idiopathic pulmonary fibrosis. The methods, compositions, and uses of the invention are useful for reducing the severity or progression of ILDs, such as idiopathic pulmonary fibrosis. Macrophages play a significant role in stimulating interstitial cells, particularly fibroblasts, to lay down collagen. Without wishing to be bound by theory, it is believed that neutrophils are involved in activating macrophages, and that the reduction of neutrophil levels found in the studies reported herein demonstrate that the methods and uses of the invention will also be applicable to reducing the severity and progression of ILDs.

In some preferred embodiments, the ILD is idiopathic pulmonary fibrosis. In other preferred embodiments, the ILD is one associated with an occupational or environmental exposure. Exemplars of such ILDs, are asbestosis, silicosis, coal worker's pneumoconiosis, and berylliosis. Further, occupational exposure to any of a number of inorganic dusts and organic dusts is believed to be associated with mucus hypersecretion and respiratory disease, including cement dust, coke oven emissions, mica, rock dusts, cotton dust, and grain dust (for a more complete list of occupational dusts associated with these conditions, see Table 254-1 of Speizer, “Environmental Lung Diseases,” Harrison's Principles of Internal Medicine, infra, at pp. 1429-1436). In other embodiments, the ILD is sarcoidosis of the lungs. ILDs can also result from radiation in medical treatment, particularly for breast cancer, and from connective tissue or collagen diseases such as rheumatoid arthritis and systemic sclerosis. It is believed that the methods, uses and compositions of the invention can be useful in each of these interstitial lung diseases.

In another set of embodiments, the invention is used to reduce the severity or progression of asthma. Asthma typically results in mucin hypersecretion, resulting in partial airway obstruction. Additionally, irritation of the airway results in the release of mediators which result in airway obstruction. While the lymphocytes and other immunomodulatory cells recruited to the lungs in asthma may differ from those recruited as a result of COPD or an ILD, it is expected that the invention will reduce the influx of immunomodulatory cells, such as neutrophils and eosinophils, and ameliorate the extent of obstruction. Thus, it is expected that the administration of sEH inhibitors, and the administration of sEH inhibitors in combination with EETs, will be useful in reducing airway obstruction due to asthma.

In each of these diseases and conditions, it is believed that at least some of the damage to the lungs is due to agents released by neutrophils which infiltrate into the lungs. The presence of neutrophils in the airways is thus indicative of continuing damage from the disease or condition, while a reduction in the number of neutrophils is indicative of reduced damage or disease progression. Thus, a reduction in the number of neutrophils in the airways in the presence of an agent is a marker that the agent is reducing damage due to the disease or condition, and is slowing the further development of the disease or condition. The number of neutrophils present in the lungs can be determined by, for example, bronchoalveolar lavage.

### Prophylactic and Therapeutic Methods to Reduce Stroke Damage:

Inhibitors of soluble epoxide hydrolase (“sEH”) and EETs administered in conjunction with inhibitors of sEH have been shown to reduce brain damage from strokes. Based on these results, we expect that inhibitors of sEH taken prior to an ischemic stroke will reduce the area of brain damage and will likely reduce the consequent degree of impairment. The reduced area of damage should also be associated with a faster recovery from the effects of the stroke.

While the pathophysiologies of different subtypes of stroke differ, they all cause brain damage. Hemorrhagic stroke differs from ischemic stroke in that the damage is largely due to compression of tissue as blood builds up in the confined space within the skull after a blood vessel ruptures, whereas in ischemic stroke, the damage is largely due to loss of oxygen supply to tissues downstream of the blockage of a blood vessel by a clot. Ischemic strokes are divided into thrombotic strokes, in which a clot blocks a blood vessel in the brain, and embolic strokes, in which a clot formed elsewhere in the body is carried through the blood stream and blocks a vessel there. In both hemorrhagic stroke and ischemic stroke, the damage is due to the death of brain cells. Based on the results observed in our studies, we would expect at least some reduction in brain damage in all types of stroke and in all subtypes.

A number of factors are associated with an increased risk of stroke. Given the results of the studies underlying the present invention, sEH inhibitors administered to persons with any one or more of the following conditions or risk factors: high blood pressure, tobacco use, diabetes, carotid artery disease, peripheral artery disease, atrial fibrillation, transient ischemic attacks (TIAs), blood disorders such as high red blood cell counts and sickle cell disease, high blood cholesterol, obesity, alcohol use of more than one drink a day for women or two drinks a day for men, use of cocaine, a family history of stroke, a previous stroke or heart attack, or being elderly, will reduce the area of brain damaged by a stroke. With respect to being elderly, the risk of stroke increases for every 10 years. Thus, as an individual reaches 60, 70, or 80, administration of sEH inhibitors has an increasingly larger potential benefit. As noted in the next section, the administration of EETs in combination with one or more sEH inhibitors can be beneficial in further reducing the brain damage.

In some preferred uses and methods, the sEH inhibitors and, optionally, EETs, are administered to persons who use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

Clot dissolving agents, such as tissue plasminogen activator (tPA), have been shown to reduce the extent of damage from ischemic strokes if administered in the hours shortly after a stroke. For example, tPA is approved by the FDA for use in the first three hours after a stroke. Thus, at least some of the brain damage from a stoke is not instantaneous, but rather occurs over a period of time or after a period of time has elapsed after the stroke. It is contemplated that administration of sEH inhibitors, optionally with EETs, can also reduce brain damage if administered within 6 hours after a stroke has occurred, more preferably within 5, 4, 3, or 2 hours after a stroke has occurred, with each successive shorter interval being more preferable. Even more preferably, the inhibitor or inhibitors are administered 2 hours or less or even 1 hour or less after the stroke, to maximize the reduction in brain damage. Persons of skill are well aware of how to make a diagnosis of whether or not a patient has had a stroke. Such determinations are typically made in hospital emergency rooms, following standard differential diagnosis protocols and imaging procedures.

In some preferred uses and methods, the sEH inhibitors and, optionally, EETs, are administered to persons who have had a stroke within the last 6 hours who: use tobacco, have carotid artery disease, have peripheral artery disease, have atrial fibrillation, have had one or more transient ischemic attacks (TIAs), have a blood disorder such as a high red blood cell count or sickle cell disease, have high blood cholesterol, are obese, use alcohol in excess of one drink a day if a woman or two drinks a day if a man, use cocaine, have a family history of stroke, have had a previous stroke or heart attack and do not have high blood pressure or diabetes, or are 60, 70, or 80 years of age or more and do not have hypertension or diabetes.

### Metabolic Syndrome

Inhibitors of soluble epoxide hydrolase (“sEH”) and EETs administered in conjunction with inhibitors of sEH have been shown to treat one or more conditions associated with metabolic syndrome as provided for in U.S. Provisional Application Ser. No. 60/887,124 which is incorporated herein by reference in its entirety.

Metabolic syndrome is characterized by a group of metabolic risk factors present in one person. The metabolic risk factors include central obesity (excessive fat tissue in and around the abdomen), atherogenic dyslipidemia (blood fat disorders—mainly high triglycerides and low HDL cholesterol), insulin resistance or glucose intolerance, prothrombotic state (e.g., high fibrinogen or plasminogen activator inhibitor in the blood), and high blood pressure (130/85 mmHg or higher).

Metabolic syndrome, in general, can be diagnosed based on the presence of three or more of the following clinical manifestations in one subject:
a) Abdominal obesity characterized by a elevated waist circumference equal to or greater than 40 inches (102 cm) in men and equal to or greater than 35 inches (88 cm) in women;
b) Elevated triglycerides equal to or greater than 150 mg/dL;
c) Reduced levels of high-density lipoproteins of less than 40 mg/dL in women and less than 50 mg/dL in men;
d) High blood pressure equal to or greater than 130/85 mm Hg; and
e) Elevated fasting glucose equal to or greater than 100 mg/dL.

It is desirable to provide early intervention to prevent the onset of metabolic syndrome so as to avoid the medical complications brought on by this syndrome. Prevention or inhibition of metabolic syndrome refers to early intervention in subjects predisposed to, but not yet manifesting, metabolic syndrome. These subjects may have a genetic disposition associated with metabolic syndrome and/or they may have certain external acquired factors associated with metabolic syndrome, such as excess body fat, poor diet, and physical inactivity. Additionally, these subjects may exhibit one or more of the conditions associated with metabolic syndrome. These conditions can be in their incipient form.

Accordingly, one aspect, the invention provides a method for inhibiting the onset of metabolic syndrome by administering to the subject predisposed thereto an effective amount of a sEH inhibitor.

Another aspect provides a method for treating one or more conditions associated with metabolic syndrome in a subject where the conditions are selected from incipient diabetes, obesity, glucose intolerance, high blood pressure, elevated serum cholesterol, and elevated triglycerides. This method comprises administering to the subject an amount of an sEH inhibitor effective to treat the condition or conditions manifested in the subject. In one embodiment of this aspect, two or more of the noted conditions are treated by administering to the subject an effective amount of an sEH inhibitor. In this aspect, the conditions to be treated include treatment of hypertension.

sEH inhibitors are also useful in treating metabolic conditions comprising obesity, glucose intolerance, hypertension, high blood pressure, elevated levels of serum cholesterol, and elevated levels of triglycerides, or combinations thereof, regardless if the subject is manifesting, or is predisposed to, metabolic syndrome.

Accordingly, another aspect of the invention provides for methods for treating a metabolic condition in a subject, comprising administering to the subject an effective amount of a sEH inhibitor, wherein the metabolic condition is selected from the group consisting of conditions comprising obesity, glucose intolerance, high blood pressure, elevated serum cholesterol, and elevated triglycerides, and combinations thereof.

In general, levels of glucose, serum cholesterol, triglycerides, obesity, and blood pressure are well known parameters and are readily determined using methods known in the art.

Several distinct categories of glucose intolerance exist, including for example, type 1 diabetes mellitus, type 2 diabetes mellitus, gestational diabetes mellitus (GDM), impaired glucose tolerance (IGT), and impaired fasting glucose (IFG). IGT and IFG are transitional states from a state of normal glycemia to diabetes. IGT is defined as two-hour glucose levels of 140 to 199 mg per dL (7.8 to 11.0 mmol) on the 75-g oral glucose tolerance test (OGTT), and IFG is defined as fasting plasma glucose (FG) values of 100 to 125 mg per dL (5.6 to 6.9 mmol per L) in fasting patients. These glucose levels are above normal but below the level that is diagnostic for diabetes. Rao, et al., Amer. Fam. Phys. 69:1961-1968 (2004).

Current knowledge suggests that development of glucose intolerance or diabetes is initiated by insulin resistance and is worsened by the compensatory hyperinsulinemia. The progression to type 2 diabetes is influenced by genetics and environmental or acquired factors including, for example, a sedentary lifestyle and poor dietary habits that promote obesity. Patients with type 2 diabetes are usually obese, and obesity is also associated with insulin resistance.

“Incipient diabetes” refers to a state where a subject has elevated levels of glucose or, alternatively, elevated levels of glycosylated hemoglobin, but has not developed diabetes. A standard measure of the long term severity and progression of diabetes in a patient is the concentration of glycosylated proteins, typically glycosylated hemoglobin. Glycosylated proteins are formed by the spontaneous reaction of glucose with a free amino group, typically the N-terminal amino group, of a protein. HbA1c is one specific type of glycosylated hemoglobin (Hb), constituting approximately 80% of all glycosylated hemoglobin, in which the N-terminal amino group of the Hb A beta chain is glycosylated.

Formation of HbA1c irreversible and the blood level depends on both the life span of the red blood cells (average 120 days) and the blood glucose concentration. A buildup of glycosylated hemoglobin within the red cell reflects the average level of glucose to which the cell has been exposed during its life cycle. Thus the amount of glycosylated hemoglobin can be indicative of the effectiveness of therapy by monitoring long-term serum glucose regulation. The HbA1c level is proportional to average blood glucose concentration over the previous four weeks to three months. Therefore HbA1c represents the time-averaged blood glucose values, and is not subject to the wide fluctuations observed in blood glucose values, a measurement most typically taken in conjunction with clinical trials of candidate drugs for controlling diabetes.

Obesity can be monitored by measuring the weight of a subject or by measuring the Body Mass Index (BMI) of a subject. BMI is determined by dividing the subject's weight in kilograms by the square of his/her height in metres (BMI=kg/m2). Alternatively, obesity can be monitored by measuring percent body fat. Percent body fat can be measured by methods known in the art including by weighing a subject underwater, by a skinfold test, in which a pinch of skin is precisely measured to determine the thickness of the subcutaneous fat layer, or by bioelectrical impedance analysis.

### Combination Therapy

As noted above, the compounds of the present invention will, in some instances, be used in combination with other therapeutic agents to bring about a desired effect. Selection of additional agents will, in large part, depend on the desired target therapy (see, e.g., Turner, N. et al. Prog. Drug Res. (1998) 51: 33-94; Haffner, S. Diabetes Care (1998) 21: 160-178; and DeFronzo, R. et al. (eds), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W., (ed), Current Therapy In Endocrinology And Metabolism, 6th Edition (Mosby-Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). Combination therapy includes administration of a single pharmaceutical dosage formulation which contains a compound of Formula (I′), (I), (Ia)-(If) or of Tables 1 or 2 and one or more additional active agents, as well as administration of the compound and each active agent in its own separate pharmaceutical dosage formulation. For example, a compound of Formula (I′), (I), (Ia)-(If), (II), or of Tables 1 or 2 and one or more angiotensin receptor blockers, angiotensin converting enzyme inhibitors, calcium channel blockers, diuretics, alpha blockers, beta blockers, centrally acting agents, vasopeptidase inhibitors, renin inhibitors, endothelin receptor agonists, AGE (advanced glycation end-products) crosslink breakers, sodium/potassium ATPase inhibitors, endothelin receptor agonists, endothelin receptor antagonists, angiotensin vaccine, and the like; can be administered to the human subject together in a single oral dosage composition, such as a tablet or capsule, or each agent can be administered in separate oral dosage formulations. Where separate dosage formulations are used, the compound of Formula (I′), (I), (Ia)-(If), (II), or of Tables 1 or 2 and one or more additional active agents can be administered at essentially the same time (i.e., concurrently), or at separately staggered times (i.e., sequentially). Combination therapy is understood to include all these regimens.

### Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day. All of these factors are within the skill of the attending clinician.

Therapeutically effective amounts of the compounds may range from approximately 0.05 to 50 mg per kilogram body weight of the recipient per day; preferably about 0.1-25 mg/kg/day, more preferably from about 0.5 to 10 mg/kg/day. Thus, for administration to a 70 kg person, the dosage range would most preferably be about 35-70 mg per day.

In general, compounds of this invention will be administered as pharmaceutical compositions by any one of the following routes: oral, systemic (e.g., transdermal, intranasal or by suppository), parenteral (e.g., intramuscular, intravenous or subcutaneous), or intrathecal administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation. This is an effective method for delivering a therapeutic agent directly to the respiratory tract (see U.S. Pat. No. 5,607,915).

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the invention in combination with at least one pharmaceutically acceptable excipient. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of the compound of based on the total formulation, with the balance being one or more suitable pharmaceutical excipients. Preferably, the compound is present at a level of about 1-80 wt %. Representative pharmaceutical formulations containing a compound of Formula (I′), (I), (Ia)-(If), (II), or of Tables 1 or 2 are described below.

### General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

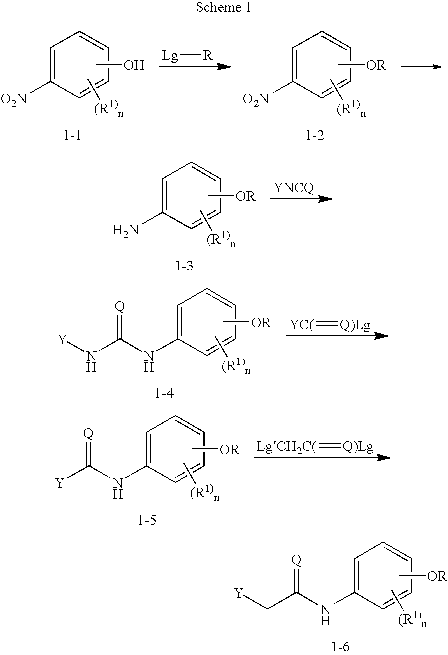

A synthesis of the compounds of the invention is shown in Scheme 1, where Q, Y, R, R¹, and n are previously defined, and where Lg and Lg′ are leaving groups such as halogen. In addition, R can be chemically modified to synthesize various compounds of the invention. For example, when R is a carboxyl ester, soponification can yield the corresponding compounds wherein R is carboxillic acid. Phenol 1-1 is treated with the appropriate compound Lg-R under suitable S_{N}2 or S_{N}Ar displacement conditions to form ether 1-2. When Lg is OH, 1-2 can be formed under Mitsunobu conditions. The resulting compound 1-2 is then reduced to the amine to form 1-3. Suitable reducing agents to effect this transformation include hydrogenation in the precense of a catalyst such as Ni or Pd or treatment of 1-2 with iron and an acid such as ammonium formate.

Compound 1-3 can be used as a starting material to form a variety of compounds having a urea, thiourea, or amide linkage. Reaction of 1-3 with isocyanate or isothiocyanate YNCQ gives the corresponding urea or thiourea 1-4. Typically, the preparation of the urea is conducted using a polar solvent such as DMF (dimethylformamide) or ethanol at 60 to 85° C.

Amides 1-5 and 1-6 can be formed by reacting 1-3 with YC(═O)Lg where Lg is a leaving group or OH or reacting 1-3 with Lg′CH₂C(═O)Lg where Lg and Lg′ are leaving groups such as halogen under amide forming conditions to give the respective amides 1-4 and 1-5.

When Lg is OH, a variety of amide coupling reagents may be used to from the amide bond, including the use of carbodiimides such as N—N′-dicyclohexylcarbodiimide (DCC), N—N′-diisopropylcarbodiimide (DIPCDI), and 1-ethyl-3-(3′-dimethylaminopropyl)carbodiimide (EDCI). The carbodiimides may be used in conjunction with additives such as dimethylaminopyridine (DMAP) or benzotriazoles such as 7-aza-1-hydroxybenzotriazole (HOAt), 1-hydroxybenzotriazole (HOBt), and 6-chloro-1-hydroxybenzotriazole (Cl-HOBt).

Amide coupling reagents also include amininum and phosphonium based reagents. Aminium salts include N-[(dimethylamino)-1H-1,2,3-triazolo[4,5-b]pyridine-1-ylmethylene]-N-methylmethanaminium hexafluorophosphate N-oxide (HATU), N-[(1H-benzotriazol-1-yl)(dimethylamino) methylene]-N-methylmethanaminium hexafluorophosphate N-oxide (HBTU), N-[(1H-6-chlorobenzotriazol-1-yl)(dimethylamino)methylene]-N-methylmethanaminium hexafluorophosphate N-oxide (HCTU), N-[(1H-benzotriazol-1-yl)(dimethylamino)methylene]-N-methylmethanaminium tetrafluoroborate N-oxide (TBTU), and N-[(1H-6-chlorobenzotriazol-1-yl)(dimethylamino)methylene]-N-methylmethanaminium tetrafluoroborate N-oxide (TCTU). Phosphonium salts include 7-azabenzotriazol-1-yl-N-oxy-tris(pyrrolidino)phosphonium hexafluorophosphate (PyAOP) and benzotriazol-1-yl-N-oxy-tris(pyrrolidino)phosphonium hexafluorophosphate (PyBOP). Amide formation step may be conducted in a polar solvent such as dimethylformamide (DMF) and may also include an organic base such as diisopropylethylamine (DIEA) or dimethylaminopyridine (DMAP).

The following examples are provided to illustrate certain aspects of the present invention and to aid those of skill in the art in practicing the invention. These examples are in no way to be considered to limit the scope of the invention.

### EXAMPLES

The examples below as well as throughout the application, the following abbreviations have the following meanings. If not defined, the terms have their generally accepted meanings.
aq.=aqueous
Boc=tert-Butoxycarbonyl
brs=broad singlet
d=doublet
DCM=dichloromethane
DIEA=diisopropylethylamine
DMAP=dimethylaminopyridine
DMF=dimethylformamide
DMSO=dimethylsulfoxide
eq or equiv=equivalent
EtOAc=ethyl acetate
g=gram
LCMS=liquid chromatography mass spectroscopy
m=multiplet
MeOH=methanol
mg=milligram
MHz=megahertz
mL=milliliter
mM=Millimolar
mmol=Millimole
m.p.=melting point
MS=Mass spectroscopy
psi=pounds per square inch
rt=room temperature
m.p.=melting point
N=normal
s=singlet
t=triplet
sat=Saturated
TEA=triethylamine
TLC=thin layer chromatography
THF=tetrahydrofuran
μL=Microliters

### Example 1

### 1-[4-(3-morpholin-4-yl-propoxy)cyclohexyl]-3-phenylurea (62)

### Synthesis of 4-(3-bromopropyl)morpholine (A-2)

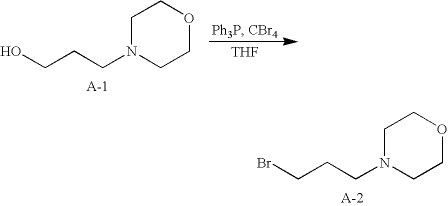

3-Morpholin-4-yl-propan-1-ol (A-1) (2.01 g, 13.9 mmol) and triphenylphosphine (3.89 g, 14.8 mmol) were dissolved in THF (29 mL) and the reaction mixture chilled in an ice-bath (0° C.) under a N₂ atmosphere. Carbon tetrabromide (4.92 g, 14.8 mmol) was added in portions over ca. 15 minutes. After ca. 30 minutes, the mixture was allowed to warm to ambient temperature. After ca. 18 h, the reaction was quenched with H₂O (10 mL) and Et₂O (30 mL). The layers were separated and the organic layer was extracted with 1 N HCl (2×ca. 15 mL). The pH of the combined aqueous extracts was adjusted to 10-11 with 4 N NaOH. The aqueous phase was extracted with EtOAc (3×ca. 30 mL), dried over Na₂SO₄, and evaporated to provide 4-(3-bromopropyl)morpholine (A-2) as a tan solid (2.53 g, 87%): ¹H NMR indicated a purity of 72% (mixture of bromide A-2 and triphenylphosphine oxide). The crude material was used in the next step without additional purification.

### Synthesis of 1-[4-(3-morpholin-4-yl-propoxy)cyclohexyl]-3-phenylurea (62)

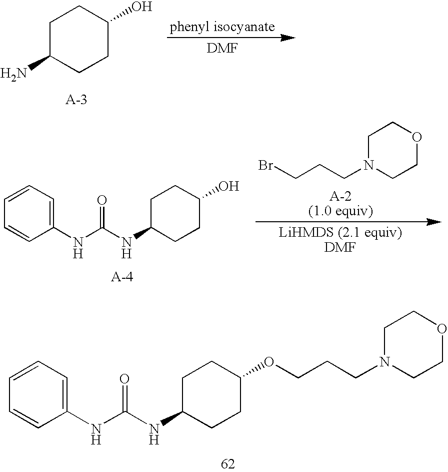

Phenyl isocyanate (0.146 mL, 1.34 mmol) was added to a mixture of trans-4-aminocyclohexanol (A-3) (163 mg, 1.42 mmol) in DMF (3.0 mL). The mixture was stirred at ambient temperature and monitored by LCMS. After 1 h the reaction was complete as determined by LCMS analysis. The mixture was chilled in an ice bath and quenched with H₂O (ca. 10 mL) and 1 N HCl (ca. 3 mL). After 30 minutes, the white solid that precipitated was collected by filtration, washed with H₂O, and transferred to a round-bottom flask with the aid of MeCN. The MeCN was evaporated and the solid was dried under high vacuum to provide 1-(4-hydroxycyclohexyl)-3-phenylurea (A-4) as a white solid (299 mg, 95%): 100% purity (% AUC at UV 214 nm): LCMS m/z 235.1 [M+H]⁺.

The crude phenyl urea (A-4) (299 mg, 1.28 mmol) was dissolved in dry DMF (3.2 mL). LiHMDS (1.0 M in THF, 2.70 mL) was added dropwise. Additional DMF (3 mL) was added to form a slurry. Crude bromide A-2 (368 mg, ca. 1.27 mmol) was added as a solid in one portion. After stirring for 20 h at ambient temperature, the THF was removed by rotary evaporation and the reaction was quenched with H₂O (ca. 20 mL) and EtOAc. LCMS analysis indicated little or no product was present in the EtOAc layer. The aqueous layer was extracted with 10% isopropanol in chloroform (4×ca. 10 mL). The combined organic layers were washed with brine (ca. 10 mL), dried over Na₂SO₄, and evaporated to provide crude 1-[4-(3-morpholin-4-yl-propoxy)cyclohexyl]-3-phenylurea (62) as a tan oil (253 mg): LCMS m/z 362.5 [M+H]⁺. LCMS indicated the presence of two mono-alkylated products (one major). The crude material was purified by reverse-phase HPLC.

### Example 2

### 2-adamantan-1-yl-N-[4-(3-morpholin-4-yl-propoxy)cyclohexyl]acetamide (50)

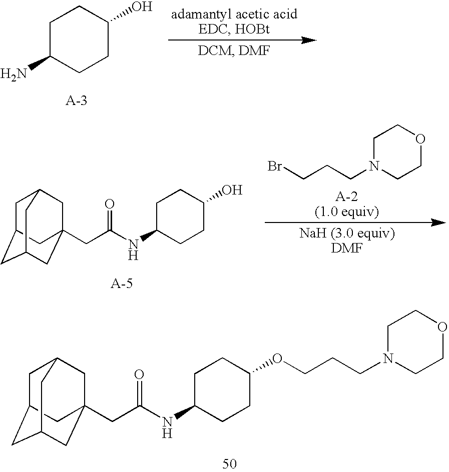

Adamantan-1-yl-acetic acid (218 mg, 1.12 mmol), HOBt.H₂O (172 mg, 1.12 mmol), and EDC.HCl (323 mg, 1.68 mmol) were combined in CH₂Cl₂ (5.0 mL). trans-4-Aminocyclohexanol (A-3) (135 mg, 1.17 mmol) was added in one portion. DMF (2 mL) was added to dissolve the aminocyclohexanol, and the mixture was stirred at ambient temperature. After 3 h, the reaction was complete by LCMS analysis. The CH₂Cl₂ was removed by rotary evaporation, and the residue was dissolved in EtOAc (ca. 20 mL). The EtOAc was washed with 1 M HCl (1×10 mL), 0.5 M NaOH (2×5 mL), and brine (1×5 mL). The EtOAc layer was dried over Na₂SO₄ and evaporated to provide 2-adamantan-1-yl-N-(4-hydroxycyclohexyl)acetamide (A-5) (228 mg, 70%): LCMS m/z 292.4 [M+H]⁺.

The crude amide A-5 (228 mg, 0.784 mmol) was dissolved in dry DMF (4.0 mL) and added to NaH (60% dispersion in mineral oil, 78 mg, 2.0 mmol). After 5 minutes, crude bromide A-2 (238 mg, ca. 0.82 mmol) was added in one portion. After stirring for 18 h at ambient temperature, the reaction was quenched with H₂O (ca. 15 mL) and EtOAc (20 mL). The EtOAc layer was washed with H₂O (2×ca. 5 mL) and brine (ca. 5 mL), dried over Na₂SO₄, and evaporated. The residue was dissolved in MeCN (2 mL) and washed with hexanes (3×1 mL) to remove the mineral oil. The MeCN was evaporated to provide crude 2-adamantan-1-yl-N-[4-(3-morpholin-4-yl-propoxy)cyclohexyl]acetamide (50) as a colorless oil (334 mg): LCMS m/z 419.42 [M+H]⁺. The crude material was purified by reverse-phase HPLC.

### Example 3

### 1-[3-(3-Morpholin-4-yl-propoxy)cyclohexyl]-3-phenylurea (63)

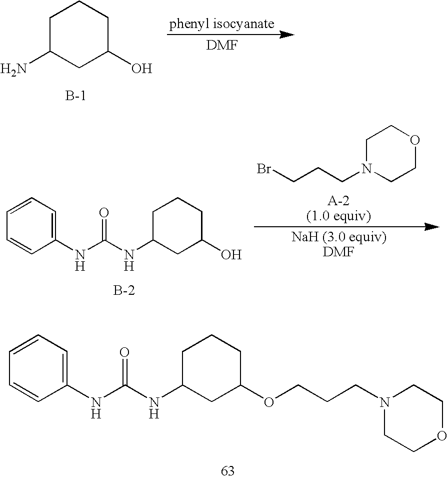

Phenyl isocyanate (0.117 mL, 1.08 mmol) was added to a mixture of 3-aminocyclohexanol (B-1) (131 mg, 1.13 mmol) in DMF (2.5 mL). The mixture was stirred at ambient temperature and monitored by LCMS. After 1.5 h, the reaction was complete as determined by LCMS analysis. The mixture was chilled in an ice bath, quenched with H₂O (ca. 5 mL) and 1 N HCl (ca. 3 mL), and extracted into EtOAc (2×ca. 10 mL). The EtOAc layers were washed with H₂O (2×10 mL) and brine (5 mL), dried over Na₂SO₄, and concentrated to provide 1-(3-hydroxycyclohexyl)-3-phenylurea (B-2) as a white solid (174 mg, 69%). LCMS m/z 235.2 [M+H]⁺.

The crude phenyl urea B-2 (174 mg, 0.744 mmol) was dissolved in dry DMF (4.0 mL) and added to NaH (60% dispersion in mineral oil, 89 mg, 2.2 mmol). After 5 minutes, crude bromide A-2 (226 mg, ca. 0.78 mmol) was added in one portion. After stirring for 20 h at ambient temperature, the reaction was quenched with H₂O (ca. 15 mL) and EtOAc (20 mL) was added. The aqueous layer was extracted with additional EtOAc (2×5 mL). The EtOAc layers were extracted with 1.0 M HCl (2×ca. 15 mL). The pH of the combined aqueous layers was adjusted to 10-11 with 2 M NaOH, and the product was extracted into EtOAc (2×ca. 15 mL). The organic layers were dried over Na₂SO₄ and evaporated to provide crude 1-[3-(3-morpholin-4-yl-propoxy)cyclohexyl]-3-phenylurea (63) as a tan oil (190 mg): LCMS m/z 362.45 [M+H]⁺. LCMS analysis indicated the presence of at least two mono-alkylated products (one major). The crude material was purified by reverse-phase HPLC.

### Example 4

### 1-adamantan-1-yl-3-[3-(3-morpholin-4-yl-propoxy)cyclohexyl]urea (48)

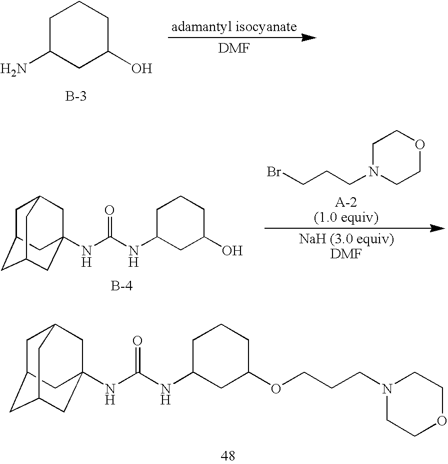

1-Isocyanato-adamantane (180 mg, 1.02 mmol) was added to a mixture of 3-aminocyclohexanol (B-3) (123 mg, 1.07 mmol) in DMF (2.5 mL). The mixture was stirred at ambient temperature and monitored by LCMS. After 1.5 h, the reaction was determined to be complete by LCMS analysis. The mixture was chilled in an ice bath and quenched with H₂O (ca. 5 mL) and 1 N HCl (ca. 3 mL). After 30 minutes, the white solid that formed was collected by filtration, washed with H₂O (ca. 20 mL), and transferred to a round-bottom flask with the aid of MeCN. The MeCN was evaporated and the solid was dried under high vacuum to provide 1-(3-Hydroxycyclohexyl)-3-phenylurea (B-4) as a white solid (220 mg, 74%): LCMS m/z 293.4 [M+H]⁺.

The crude urea B-4 (220 mg, 0.751 mmol) was dissolved in dry DMF (4.0 mL) and added to NaH (60% dispersion in mineral oil, 90 mg, 2.2 mmol). After 5 minutes, crude bromide A-2 (228 mg, ca. 0.789 mmol) was added in one portion. After stirring for 20 h at ambient temperature, the reaction was quenched with H₂O (ca. 15 mL) and EtOAc (20 mL) was added. The EtOAc layer was washed with H₂O (2×ca. 5 mL) and brine (ca. 5 mL), dried over Na₂SO₄, and evaporated. The residue was dissolved in MeCN (ca. 2 mL) and washed with hexanes (3×1 mL) to remove the mineral oil. The MeCN was evaporated to provide crude 1-adamantan-1-yl-3-[3-(3-morpholin-4-yl-propoxy)cyclohexyl]urea (48) as a tan oil (298 mg): LCMS m/z 420.48 [M+H]⁺. The crude material was purified by reverse-phase HPLC.

### Example 5

### 2-adamantan-1-yl-N-[3-(3-morpholin-4-yl-propoxy)cyclohexyl]acetamide (49)

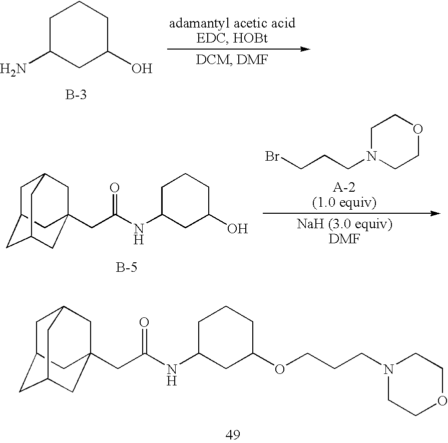

2-Adamantan-1-yl-N-[3-(3-morpholin-4-yl-propoxy)cyclohexyl]acetamide (49) was prepared according to the procedure described for the synthesis of 2-adamantan-1-yl-N-[4-(3-morpholin-4-yl-propoxy)-cyclohexyl]acetamide (50). Adamantan-1-yl-acetic acid (278 mg, 1.43 mmol), HOBt.H₂O (219 mg, 1.43 mmol), EDC.HCl (411 mg, 2.14 mmol), and 3-aminocyclohexanol (B-3) (173 mg, 1.50 mmol) provided 2-adamantan-1-yl-N-(3-hydroxycyclohexyl)acetamide (B-5) (451 mg) were used: LCMS m/z 292.35 [M+H]⁺. The crude amide B-5 (451 mg, ca. 1.43 mmol) was treated with NaH (60% dispersion in mineral oil, 143 mg, 3.57 mmol) and bromide A-2 (413 mg, ca. 1.43 mmol) to provide 2-adamantan-1-yl-N-[3-(3-morpholin-4-yl-propoxy)cyclohexyl]acetamide (49) (522 mg): LCMS m/z 419.45 [M+H]⁺. The crude material was purified by reverse-phase HPLC.

### Example 6

### 1-cyclohexyl-3-[4-(3-morpholin-4-yl-propoxy)phenyl]urea (45)

### Synthesis of 3-morpholinopropyl methanesulfonate (E-2)

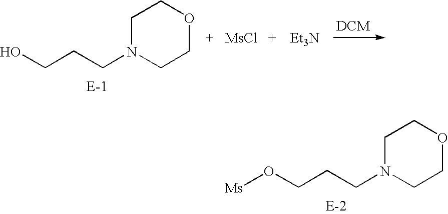

A solution of 4-(3-hydroxypropyl)morpholine (E-1) (5.08 g, 35.0 mmol, 1.0 equiv), CH₂Cl₂ (175 mL) and Et₃N (5.85 mL, 42.0 mmol, 1.2 equiv) was purged under nitrogen (2 min) and cooled to 0° C. A mixture of methane sulfonyl chloride (2.98 mL, 38.5 mmol, 1.1 equiv.) in CH₂Cl₂ (25 mL) was added dropwise over a 15 min. period. The mixture was allowed to slowly warm to rt and stirred for 3 h. The mixture was washed with water (50 mL) and brine (50 mL), dried (Na₂SO₄), filtered, and evaporated to provide 3-morpholinopropyl methanesulfonate (E-2) (7.47 g, 96%). LCMS m/z 224.1 [M+H]⁺. Due to the potential volatility of the product, it was only dried under high vacuum for 45 min. and then used in the next step without further purification.

### Synthesis of [4-(3-morpholin-4-yl-propoxy)phenyl]carbamic acid tert-butyl ester (E-4)

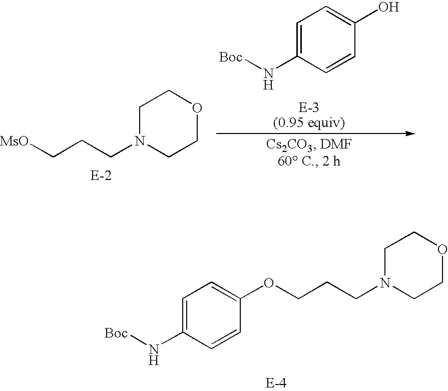

3-Morpholinopropyl methanesulfonate (E-2) (783 mg, 3.51 mmol) was dissolved in ca. 3 mL DMF and added to a mixture of (4-hydroxyphenyl)carbamic acid tert-butyl ester (E-3) (697 mg, 3.33 mmol) and cesium carbonate (2.29 g, 7.03 mmol) in DMF (5 mL). The flask was stirred under N₂ and heated in an oil bath at 60° C. (external bath temperature). The reaction was monitored by HPLC for the disappearance of the starting material E-3. After 2 h, only traces of starting material E-3 were present. The mixture was cooled to ambient temperature and quenched with H₂O (ca. 20 mL) and EtOAc (ca. 30 mL). The layers were separated and the aqueous layer was extracted with EtOAc (20 mL). The combined EtOAc extracts were washed with H₂O (2×ca. 20 mL) and brine (ca. 20 mL), dried over Na₂SO₄, filtered, evaporated, and dried under high vacuum (18 h) to provide (3-hydroxyphenyl)carbamic acid tert-butyl ester (E-4) as a tan oil (1.12 g, 99%). Purity: 100% (% AUC, UV detection@214 nm); LCMS m/z 337.30 [M+H]⁺. The crude material was used in the next step without additional purification.

### Synthesis of 4-(3-morpholin-4-yl-propoxy)phenylamine (E-5)

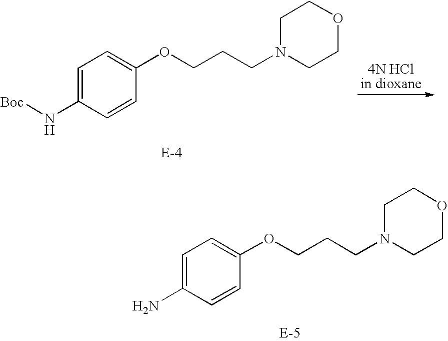

Crude (3-hydroxyphenyl)carbamic acid tert-butyl ester (E-4) (1.12 g, 3.33 mmol) was treated with 4 N HCl in 1,4-dioxane (8 mL) at ambient temperature. The biphasic mixture was allowed to stand at ambient temperature for 1 h. HPLC analysis indicated complete consumption of E-4 and formation of a new product (product eluted in the solvent front). The HCl in 1,4-dioxane solution was removed by rotary evaporation, and the residue was dissolved in a mixture of EtOAc (ca. 20 mL) and saturated NaHCO₃ (ca. 20 mL). The aqueous layer was extracted with additional EtOAc (2×ca. 15 mL). The combined EtOAc layers were dried over Na₂SO₄, and evaporated to provide crude 4-(3-morpholin-4-yl-propoxy)phenylamine (E-5) as a tan oil (841 mg, quantitative recovery). LCMS m/z 237.10 [M+H]⁺ (product eluted at solvent front). The crude material was used in the next step without additional purification.

### Synthesis of 1-cyclohexyl-3-[4-(3-morpholin-4-yl-propoxy)phenyl]urea (45)

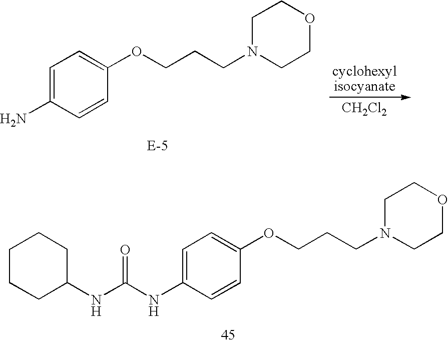

4-(3-Morpholin-4-yl-propoxy)phenylamine (E-5) (284 mg, 1.20 mmol) was dissolved in CH₂Cl₂ (3 mL). Cyclohexyl isocyanate (0.161 mL, 1.26 mmol) was added and the mixture was stirred at ambient temperature. After 18 h, LCMS analysis indicated formation of the desired product 45 (LCMS m/z 362.30 [M+H]⁺). Traces of starting material E-5 (LCMS m/z 237.10 [M+H]⁺) were observed at the solvent front. The CH₂Cl₂ was evaporated and the residue was purified by reverse-phase HPLC.

### Example 7

### 1-[4-(3-morpholin-4-yl-propoxy)phenyl]-3-phenylurea (14)

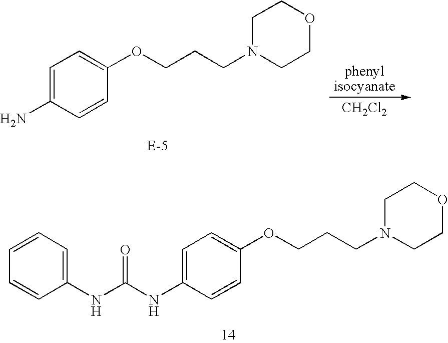

4-(3-Morpholin-4-yl-propoxy)phenylamine (E-5) (257 mg, 1.09 mmol) was dissolved in CH₂Cl₂ (3 mL). Phenyl isocyanate (0.124 mL, 1.14 mmol) was added and the mixture was stirred at ambient temperature. After 18 h, LCMS analysis indicated complete consumption of starting material E-5 and formation of desired product 14 (LCMS m/z 356.27 [M+H]⁺). The CH₂Cl₂ was evaporated and the residue was purified by reverse-phase HPLC.

### Example 8

### 2-adamantan-1-yl-N-[4-(3-morpholin-4-yl-propoxy)phenyl]acetamide (46)

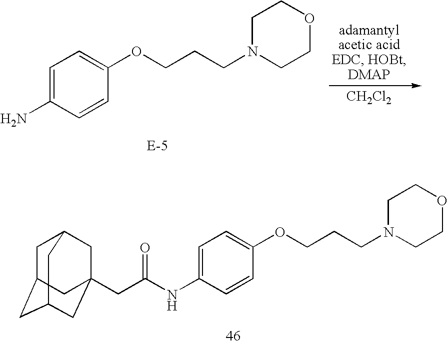

Adamantan-1-yl-acetic acid (200 mg, 1.03 mmol), HOBt.H₂O (158 mg, 1.03 mmol), and EDC.HCl (297 mg, 1.55 mmol) were combined in CH₂Cl₂ (ca. 2 mL). 4-(3-Morpholin-4-yl-propoxy)phenylamine (E-5) (244 mg, 1.03 mmol) was dissolved in CH₂Cl₂ (ca. 2 mL) and added to the activated acid solution. The mixture was stirred at ambient temperature. After 2 h, LCMS analysis indicated formation of desired product 46 (LCMS m/z 413.36 [M+H]⁺). A trace of starting material E-5 (LCMS m/z 237.10 [M+H]⁺) was observed at the solvent front. After 18 h, the CH₂Cl₂ was evaporated and the residue was purified by reverse-phase HPLC.

### Example 9

### Synthesis of 1-cyclohexyl-3-(3-(3-morpholinopropoxy)phenyl)urea (41)

### Synthesis of (3-hydroxyphenyl)carbamic acid tert-butyl ester (F-2)

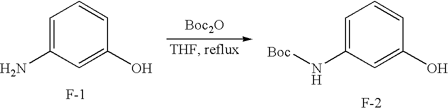

3-Aminophenol (F-1) (3.25 g, 29.8 mmol) was dissolved in THF (60 mL). Boc₂O (7.15 g, 32.8 mmol) was dissolved in THF (10 mL) and added in one portion. The mixture was heated at reflux temperature for 18 h. HPLC analysis indicated complete consumption of the aminophenol starting material and formation of a single product. The THF was evaporated and the residue was dissolved in EtOAc (100 mL). The EtOAc layer was washed with 1% aqueous HCl (ca. 30 mL), saturated NaHCO₃ (ca. 30 mL), and brine (ca. 30 mL). The EtOAc extract was dried over Na₂SO₄, filtered, and evaporated to provide (3-hydroxyphenyl)carbamic acid tert-butyl ester (F-2) as a colorless foam (7.18 g). Purity: 100% (% AUC, UV detection@214 nm); LCMS m/z 210.04 [M+H]⁺. The crude material was used in the next step without additional purification.

### Synthesis of 3-(3-morpholinopropoxy)aniline (F-4)

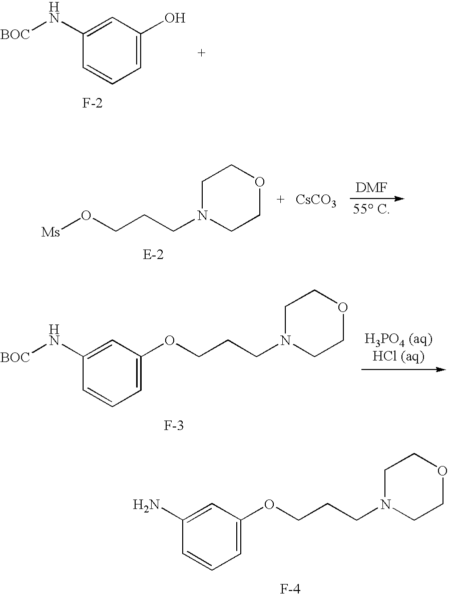

To a solution of 3-N—BOC-aminophenol (F-2) (6.65 g, 29.8 mmol, 1.0 equiv) in DMF (100 mL) was added cesium carbonate (19.4 g, 59.6 mmol, 2.0 equiv) followed by a solution of 3-morpholinopropyl methanesulfonate (E-2) (6.24 g, 29.8 mmol, 1.0 equiv) in DMF (50 mL). The mixture was stirred at 55° C. for 3 days then diluted with ethyl acetate (200 mL), and water (200 mL). The layers were separated and the aqueous layer was back extracted with EtOAc (50 mL) and combined with the first organic extract. The combined organics were washed with water (2×100 mL), 1 M NaH₂PO₄ (50 mL) and brine (100 mL). The combined NaH₂PO₄ and brine washes were back extracted with EtOAc (50 mL). Intermediate F-3 (tert-butyl 3-(3-morpholinopropoxy)phenylcarbamate) was extracted from the EtOAc into aqueous 10% H₃PO₄ (2×50 mL). Concentrated HCl (aq) (25 mL, ca. 300 mmol) was added to the phosphoric acid extracts and the mixture was stirred at rt for 2 h. Additional HCl (12 mL, ca. 150 mmol) was added and the mixture was stirred at rt for 30 minutes. The solution was basified to pH≧12 with 6 M NaOH, cooled with ice (100 g), and extracted using CH₂Cl₂ (2×50 mL), dried over Na₂SO₄, filtered, and concentrated in vacuo to provide 3-(3-morpholinopropoxy)aniline (F-4) as a tan powder (5.79 g, 82%): LCMS m/z 237.1 [M+H]⁺ (product eluted at the solvent front). The crude material was used in the next step without additional purification.

### Synthesis of 1-cyclohexyl-3-(3-(3-morpholinopropoxy)phenyl)urea (41)

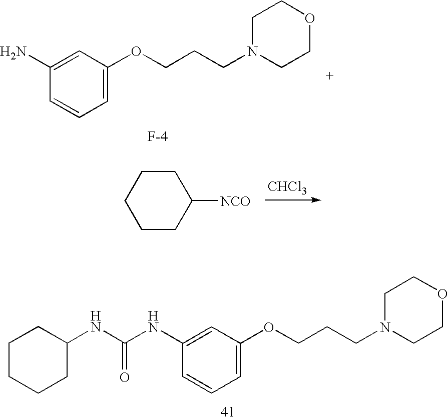

To cyclohexyl isocyanate (107 μL, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 6 days and then quenched with MeOH (ca. 1 mL). The solvent was removed in vacuo and the residue was purified by reverse-phase HPLC.

### Example 10

### 1-(3-(3-morpholinopropoxy)phenyl)-3-phenylurea (1)

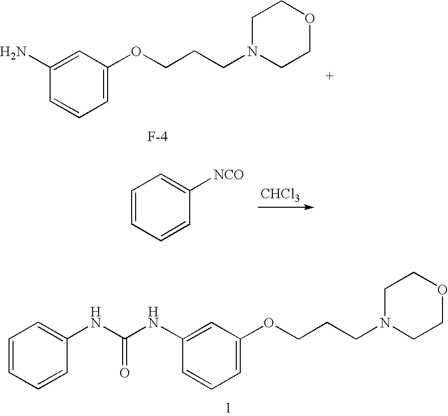

To phenyl isocyanate (91 μL, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days then quenched with MeOH (ca. 1 mL). The solvent was removed in vacuo and the residue was purified by reverse-phase HPLC.

### Example 11

### 1-adamantyl-3-(3-(3-morpholinopropoxy)phenyl)urea (47)

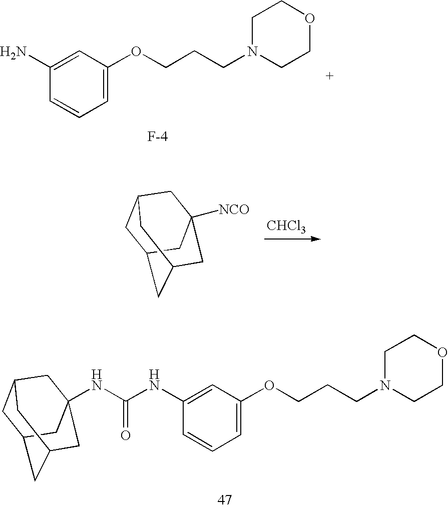

To adamantyl isocyanate (149 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 3 days followed by 3 days at 50° C. and then quenched with MeOH (ca. 1 mL). The solvent was removed in vacuo and the residue was purified by reverse-phase HPLC.

### Example 12

### 2-adamantyl-N-(3-(3-morpholinopropoxy)phenyl)acetamide (43)

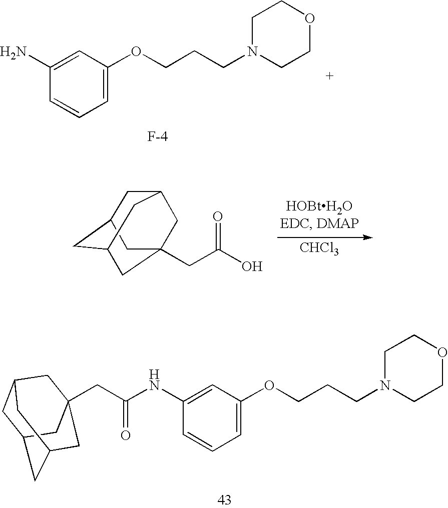

A solution of 3-(3-morpholinopropoxy)aniline (F-4) (189 mg, 0.80 mmol, 1.0 equiv) and DMAP (5 mg, 0.04 mmol, 0.05 equiv) in CHCl₃ (2.7 mL) was added to a mixture of solid HOBt-H₂O (129 mg, 0.84 mmol, 1.05 equiv) and adamantyl acetic acid (163 mg, 0.84 mmol, 1.05 equiv). The solution was stirred and EDC.HCl (184 mg, 0.96 mmol, 1.2 equiv) was then added. The resulting mixture was stirred at rt for 19 h followed by 5 h at 55° C. and then at rt for an additional 3 days. The reaction was quenched with MeOH (ca. 1 mL) and the solvent was removed in vacuo. The crude material obtained was purified by reverse-phase HPLC.

### Example 13

### 1-(4-chlorophenyl)-3-(3-(3-morpholinopropoxy)phenyl)urea (15)

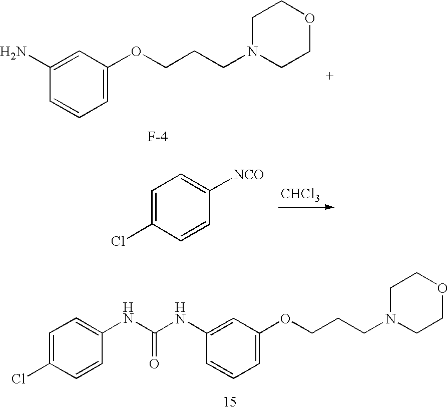

To 4-chlorophenyl isocyanate (129 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days, quenched with MeOH (ca. 1 mL), and the solvent was removed in vacuo. The crude material obtained was purified by reverse-phase HPLC.

### Example 14

### 1-(4-fluorophenyl)-3-(3-(3-morpholinopropoxy)phenyl)urea (2)

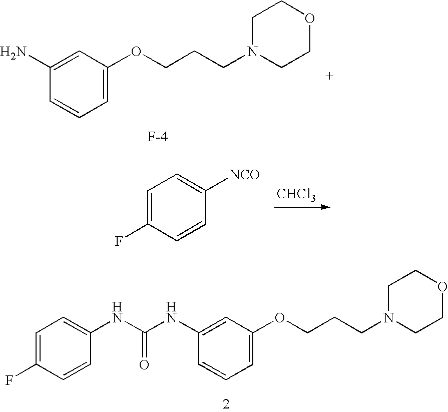

To 4-fluorophenyl isocyanate (115 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days, quenched with MeOH (ca. 1 mL), and the solvent removed in vacuo. The crude material obtained was purified by reverse-phase HPLC.

### Example 15

### 2-(4-fluorophenyl)-N-(3-(3-morpholinopropoxy)phenyl)acetamide (11)

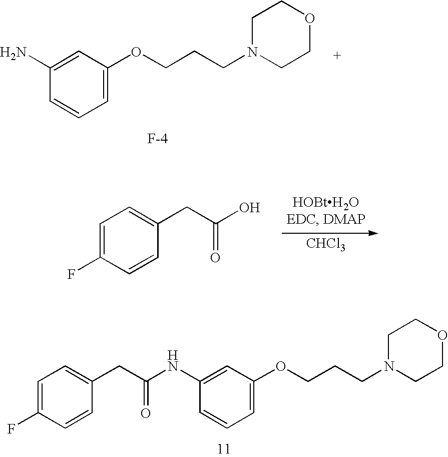

A solution of 3-(3-morpholinopropoxy)aniline (F-4) (189 mg, 0.80 mmol, 1.0 equiv) and DMAP (5 mg, 0.04 mmol, 0.05 equiv) in CHCl₃ (2.7 mL) was added to a mixture of solid HOBt.H₂O (129 mg, 0.84 mmol, 1.05 equiv) and 4-fluorophenylacetic acid (129 mg, 0.84 mmol, 1.05 equiv). The solution was stirred, EDC.HCl (184 mg, 0.96 mmol, 1.2 equiv) added, and the reaction continued at rt for 4 days. The reaction was quenched with MeOH (ca. 1 mL) and the solvent removed in vacuo. The crude material obtained was purified by reverse-phase HPLC.

### Example 16

### 1-(2,6-dichlorophenyl)-3-(3-(3-morpholinopropoxy)phenyl)urea (3)

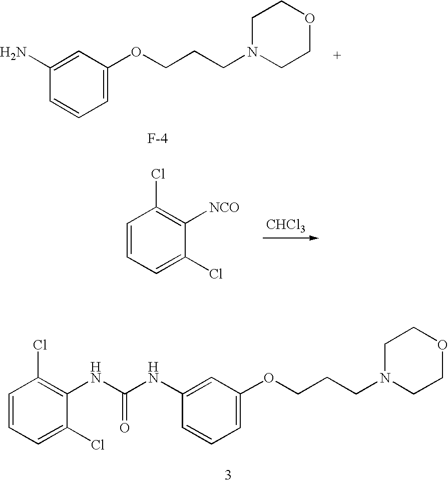

To 2,6-dichlorophenyl isocyanate (158 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days and then quenched with MeOH (ca. 1 mL). The solvent was removed in vacuo. and the crude material obtained was purified by reverse-phase HPLC.

### Example 17

### 1-(3,4-difluorophenyl)-3-(3-(3-morpholinopropoxy)phenyl)urea (4)

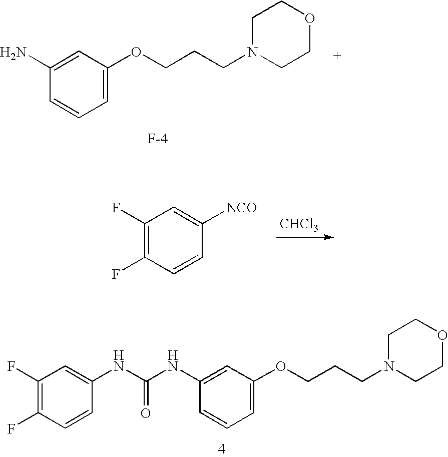

To 3,4-difluorophenyl isocyanate (130 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days, quenched with MeOH (ca. 1 mL), and the solvent removed in vacuo. The crude material obtained was purified by reverse-phase HPLC.

### Example 18

### 2-(3,4-difluorophenyl)-N-(3-(3-morpholinopropoxy)phenyl)acetamide (19)

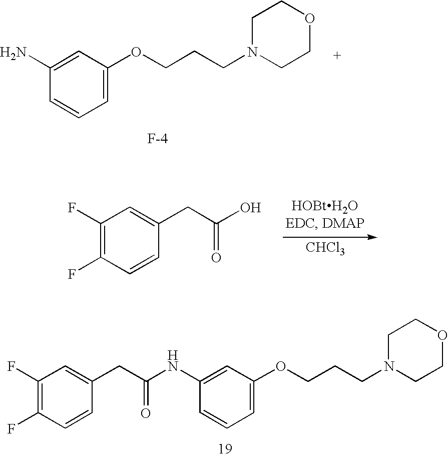

A solution of 3-(3-morpholinopropoxy)aniline (F-4) (189 mg, 0.80 mmol, 1.0 equiv) and DMAP (5 mg, 0.04 mmol, 0.05 equiv) in CHCl₃ (2.7 mL) was added to a mixture of solid HOBt-H₂O (129 mg, 0.84 mmol, 1.05 equiv) and 3,4-difluorophenylacetic acid (145 mg, 0.84 mmol, 1.05 equiv). The solution was stirred, EDC.HCl (184 mg, 0.96 mmol, 1.2 equiv) added, and the reaction continued at rt for 4 days. The reaction was quenched with MeOH (ca. 1 mL) and the solvent removed in vacuo. The crude material obtained was purified by reverse-phase HPLC.

### Example 19

### 1-(2,4-difluorophenyl)-3-(3-(3-morpholinopropoxy)phenyl)urea (5)

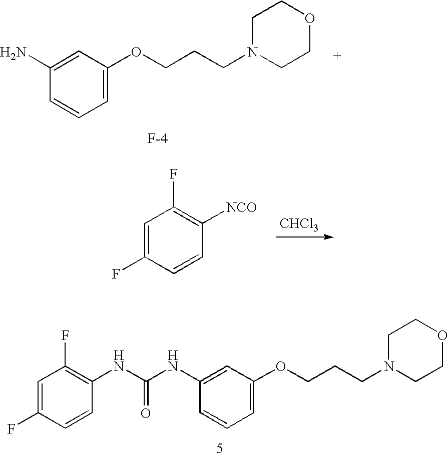

To 2,4-difluorophenyl isocyanate (130 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days, quenched with MeOH (ca. 1 mL), and the solvent was removed in vacuo. The crude material obtained was purified by reverse-phase HPLC.

### Example 20

### 1-(3-(3-morpholinopropoxy)phenyl)-3-(2,4,6-trifluorophenyl)urea (6)

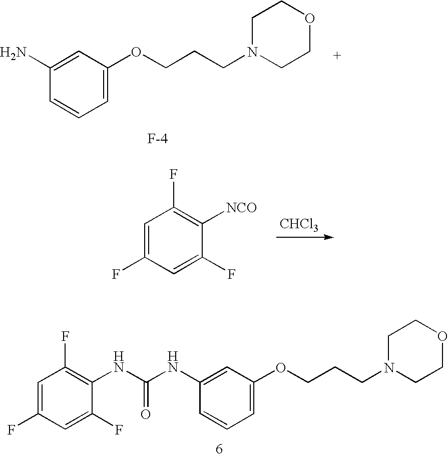

To 2,4,6-trifluorophenyl isocyanate (145, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days, quenched with MeOH (ca. 1 mL), and the solvent was removed in vacuo. The crude material obtained was purified by reverse-phase HPLC.

### Example 21

### 1-(3-(3-morpholinopropoxy)phenyl)-3-(perfluorophenyl)urea (7)

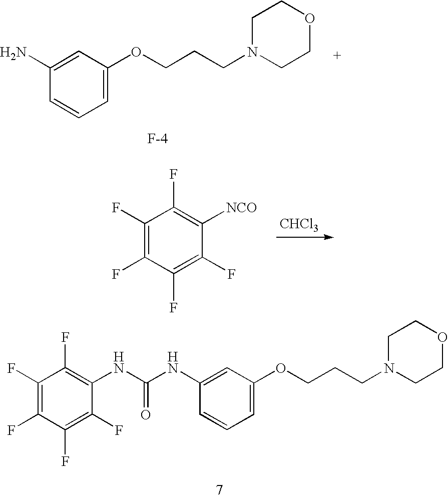

To pentafluorophenyl isocyanate (176 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days, quenched with MeOH (ca. 1 mL), and the solvent was removed in vacuo. The crude material obtained was purified by reverse-phase HPLC.

### Example 22

### 1-(benzo[d][1,3]dioxol-5-yl)-3-(3-(3-morpholinopropoxy)phenyl)urea (8)

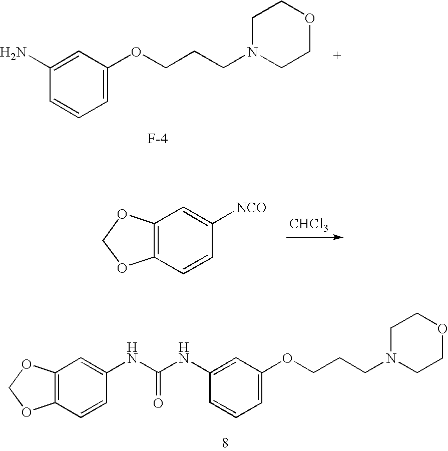

To 3,4-(methylenedioxy)phenyl isocyanate (137 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days, quenched with MeOH (ca. 1 mL), and the solvent was removed in vacuo. The crude material obtained was purified by reverse-phase HPLC.

### Example 23

### 2-(benzo[d][1,3]dioxol-5-yl)-N-(3-(3-morpholinopropoxy)phenyl)acetamide (12)

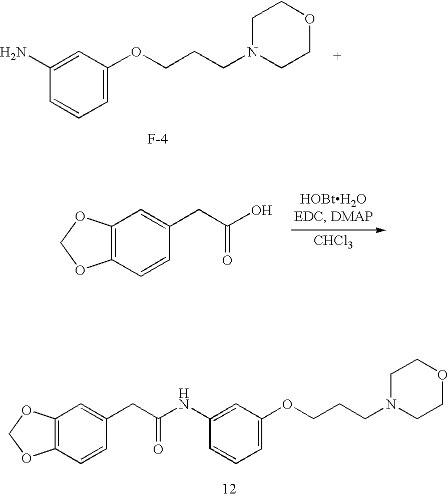

A solution of 3-(3-morpholinopropoxy)aniline (F-4) (189 mg, 0.80 mmol, 1.0 equiv) and DMAP (5 mg, 0.04 mmol, 0.05 equiv) in CHCl₃ (2.7 mL) was added to a mixture of solid HOBt-H₂O (129 mg, 0.84 mmol, 1.05 equiv) and 3,4-(methylenedioxy)phenylacetic acid (151 mg, 0.84 mmol, 1.05 equiv) followed by the addition of Et₃N (117 μL, 0.84 mmol, 1.05 equiv). The solution was stirred, EDC.HCl (184 mg, 0.96 mmol, 1.2 equiv) was added and the reaction continued at rt for 4 days. The reaction was quenched with MeOH (ca. 1 mL) and the solvent was removed in vacuo. The crude material obtained was purified by reverse-phase HPLC.

### Example 24

### 1-(3-(3-morpholinopropoxy)phenyl)-3-(pyridin-3-yl)urea(16)

### Synthesis of 4-nitrophenyl 3-(3-morpholinopropoxy)phenylcarbamate (F-6)

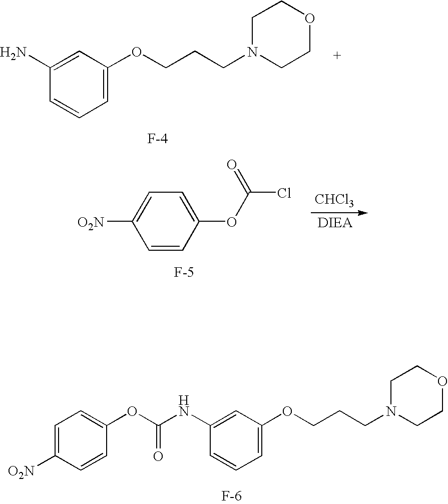

A solution of 4-nitrophenyl chloroformate (F-5) (635 mg, 3.15 mmol, 1.05 equiv) in CHCl₃ (6 mL) was purged with nitrogen and cooled to approximately −25° C. A solution of 3-(3-morpholinopropoxy)aniline (F-4) (709 mg, 3.0 mmol, 1.0 equiv) in CHCl₃ (6 mL) and diisopropylethylamine (575 μL, 3.3 mmol, 1.1 equiv) was added dropwise over a 5 min. The mixture was stirred at −25° C. for 1 min. and then at rt for 25 minutes. The solution was used immediately in the next step without workup or isolation. LCMS m/z 402.3 [M+H]⁺.

### Synthesis of 1-(3-(3-morpholinopropoxy)phenyl)-3-(pyridin-3-yl)urea (16)

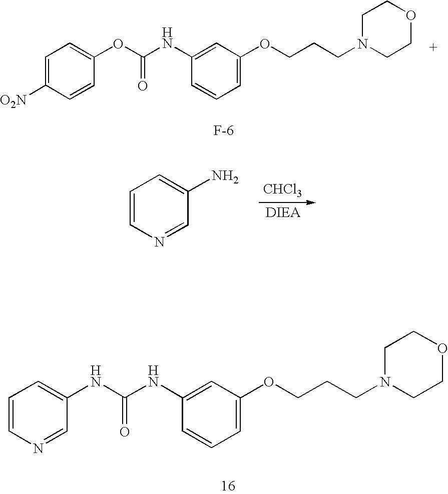

To a solution of 3-aminopyridine (99 mg, 1.05 mmol, 1.05 equiv) in CHCl₃ (1 mL) was added one third of the 4-nitrophenyl 3-(3-morpholinopropoxy)phenylcarbamate solution (F-6) (ca. 1.0 mmol, 1 equiv) obtained from the previous step. The resulting mixture was stirred at rt for 6 days. The reaction was quenched with MeOH (ca. 3 mL) and the solvent removed in vacuo. The crude material obtained was purified by reverse-phase HPLC.

### Example 25

### N-(3-(3-morpholinopropoxy)phenyl)-2-(pyridin-3-yl)acetamide (13)

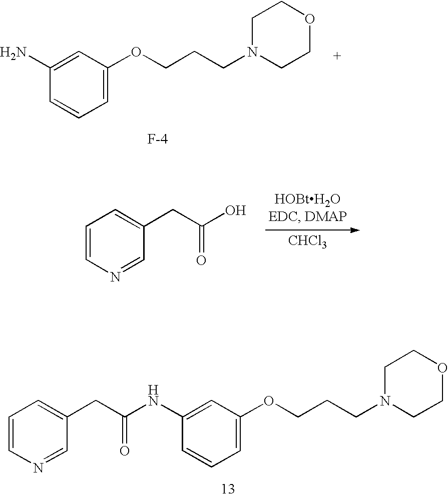

A solution of 3-(3-morpholinopropoxy)aniline (F-4) (189 mg, 0.80 mmol, 1.0 equiv) and DMAP (5 mg, 0.04 mmol, 0.05 equiv) in CHCl₃ (2.7 mL) was added to a mixture of solid HOBt.H₂O (129 mg, 0.84 mmol, 1.05 equiv) and 3-pyridylacetic acid.HCl (146 mg, 0.84 mmol, 1.05 equiv). The solution was stirred, EDC.HCl (184 mg, 0.96 mmol, 1.2 equiv) and Et₃N (117 μL, 0.84 mmol, 1.05 equiv) were added and the reaction continued at rt for 4 days. The reaction was quenched with MeOH (ca. 1 mL) and the solvent was removed in vacuo. The crude material obtained was purified by reverse-phase HPLC.

### Example 26

### 1-(3-(dimethylamino)phenyl)-3-(3-(3-morpholinopropoxy)phenyl)urea (17)

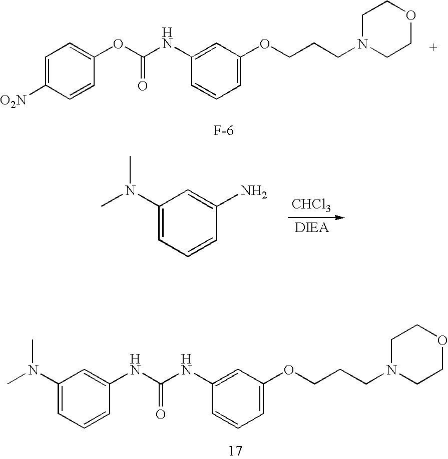

To a solution of 3-(dimethylamino)aniline.2HCl (222 mg, 1.05 mmol, 1.05 equiv) and diisopropylethylamine (366 μL, 2.10 mmol, 2.1 equiv) in CHCl₃ (1 mL) was added one third of the 4-nitrophenyl 3-(3-morpholinopropoxy)phenylcarbamate solution (F-6) (ca. 1.0 mmol, 1 equiv) prepared above and the resulting mixture was stirred at rt for 16 h. The reaction was quenched with MeOH (ca. 3 mL) and the solvent was removed in vacuo. The crude material obtained was purified by reverse-phase HPLC.

### Example 27

### 1-tert-butyl-3-(3-(3-morpholinopropoxy)phenyl)urea (42)

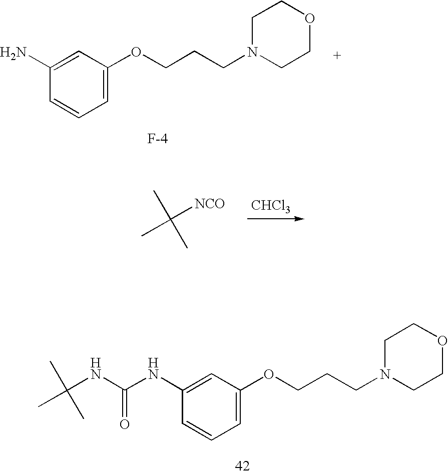

To tert-butyl isocyanate (83 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 3 days followed by 3 days at 50° C., quenched with MeOH (ca. 1 mL), and the solvent was removed in vacuo. The crude material obtained was purified by reverse-phase HPLC.

### Example 28

### 3,3-dimethyl-N-(3-(3-morpholinopropoxy)phenyl)butanamide (44)

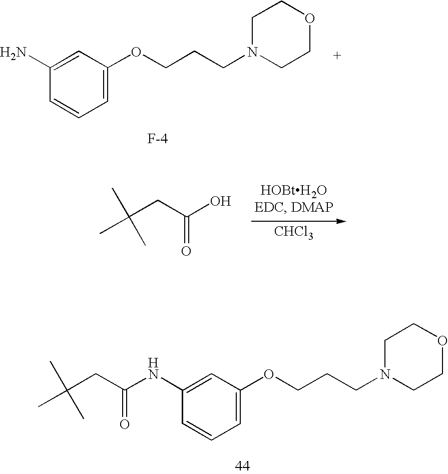

A solution of 3-(3-morpholinopropoxy)aniline (F-4) (189 mg, 0.80 mmol, 1.0 equiv) and DMAP (5 mg, 0.04 mmol, 0.05 equiv) in CHCl₃ (2.7 mL) was added to a mixture of solid HOBt.H₂O (129 mg, 0.84 mmol, 1.05 equiv) and tert-butylacetic acid (98 mg, 0.84 mmol, 1.05 equiv). The solution was stirred, EDC.HCl (184 mg, 0.96 mmol, 1.2 equiv) added and the reaction continued at rt for 19 h, 5 h at 55° C., and then at rt for 3 days. The reaction was quenched with MeOH (ca. 1 mL) and the solvent was removed in vacuo. The crude material obtained was purified by reverse-phase HPLC.

### Example 29

### 1-(3-(3-morpholinopropoxy)phenyl)-3-(4-(trifluoromethyl)phenyl)urea (9)

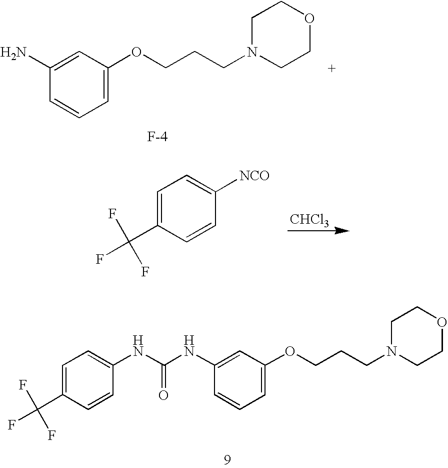

To 4-(trifluoromethyl)phenyl isocyanate (157 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days, quenched with MeOH (ca. 1 mL), and the solvent was removed in vacuo. The crude material obtained was purified by reverse-phase HPLC.

### Example 30

### 1-(3-(3-morpholinopropoxy)phenyl)-3-(3-(trifluoromethyl)phenyl)urea (10)

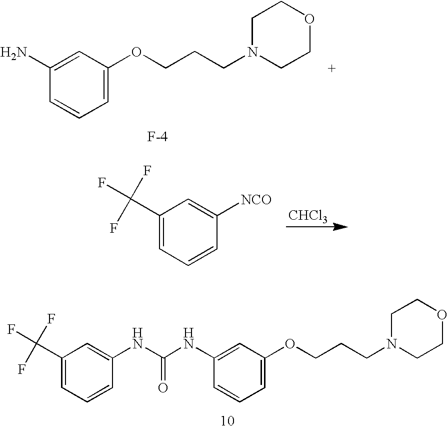

To 3-(trifluoromethyl)phenyl isocyanate (157 mg, 0.84 mmol, 1.05 equiv) was added a solution of 3-(3-morpholinopropoxy)aniline (F-4) (189 mg, 0.80 mmol, 1.0 equiv) in CHCl₃ (2.7 mL). The mixture was stirred at rt for 2 days, quenched with MeOH (ca. 1 mL), and the solvent was removed in vacuo. The crude material obtained was purified by reverse-phase HPLC.

### Example 31

### 1-(3-(2-morpholinoethoxy)phenyl)-3-(3-(3-morpholinopropoxy)phenyl)urea (18)

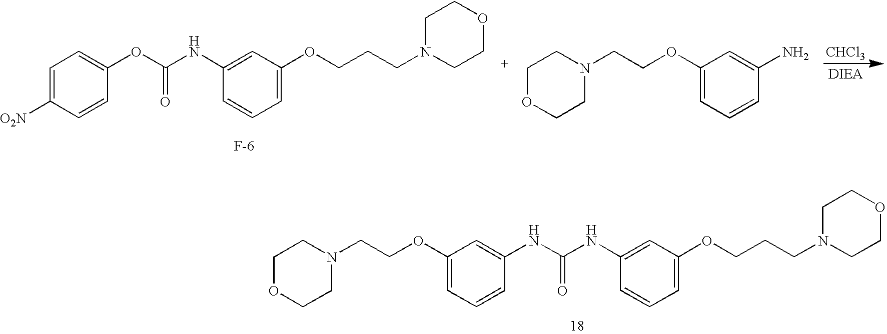

To a solution of 3-(2-morpholine-2-yl-ethoxy)phenylamine (233 mg, 1.05 mmol, 1.05 equiv) in CHCl₃ (1 mL) was added one third of the 4-nitrophenyl 3-(3-morpholinopropoxy)phenyl-carbamate solution (F-6) (ca. 1.0 mmol, 1 equiv) prepared above and the resulting mixture was stirred at rt for 16 h. The reaction was quenched with MeOH (ca. 3 mL) and the solvent was removed in vacuo. The crude material obtained was purified by reverse-phase HPLC.

### Example 32

### 1-(3-Benzyloxy-phenyl)-3-(4-fluoro-phenyl)-urea (38)

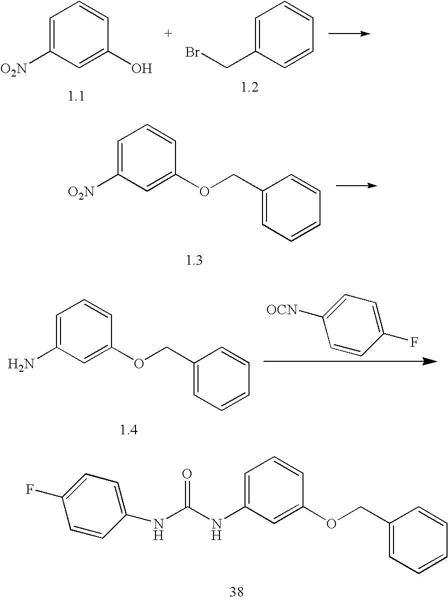

To a stirred solution of m-nitrophenol 1.1 (4.00 g, 28.7 mmol) in dry DMF was added potassium carbonate (9.90 g, 71.6 mmol), and the resulting mixture was stirred for 10 min at room temperature. Benzyl bromide 1.2 (3.5 mL, 28.3 mmol) was added to the mixture, and the temperature was slowly raised to 80° C. and maintained at this temperature for 8 hr. The progress of the reaction was monitored by TLC. On completion of the reaction, the reaction mixture was partitioned between ethyl acetate and water. The organic layer was dried over sodium sulfate, and the solvent was removed under reduced pressure to give the crude intermediate 1.3 which was purified by chromatography on silica gel (100-200 mesh) eluting with 15% ethyl acetate in hexane to afford 6.0 g of pure compound 1.3.

Compound 1.3 was then taken in a 1:1 mixture in toluene and to it was added iron powder (10 g, 28 mmol) and ammonium formate (7.0 g, 28 mmol). The reaction mixture was then heated at 80° C. for 6 hrs. The progress of the reaction was monitored by TLC. On completion of the reaction; the reaction mixture was filtered and partitioned between ethyl acetate and water. The organic layer was dried over sodium sulfate, and the solvent was removed under reduced pressure to give the crude compound which was purified by chromatography on silica gel (100-200 mesh) eluting with 25% ethyl acetate in hexane as eluent to afford 4.0 g of pure amine 1.4.

To a stirred solution of 3-benzyloxy aniline 1.4 (0.200 g, 1 mmol) in DMF at 0-5° C. was added 4-fluorophenyl isocyanate (0.14 g, 0.84 mmol), and the resulting mixture was stirred for 30 min under a nitrogen atmosphere. The progress of the reaction was monitored by TLC (10% MeOH-DCM). On completion of the reaction; the mixture was partitioned between ethyl acetate and water. The organic layer was dried over sodium sulfate, and the solvent was removed under reduced pressure to give the crude which was purified by chromatography on silica gel (100-200 mesh) eluting with 45% ethyl acetate in hexane to afford 196 mg of the title compound 38. ¹HNMR (CDCl₃): δ 8.02 (d, 2H, J=10 Hz); 7.50-7.25 (m, 7H); 7.20 (m, 1H); 7.05-7.0 (m, 2H); 6.90-6.85 (m, 2H); 6.70-6.65 (d, 1H, J=10 Hz); 5.05-5.2 (s, 2H). Mass: (M+1, 336, 100%). m.p. 187-189° C.

### Example 33

### Adamantane-1-carboxylic acid [3-(3-morpholin-4-yl-propoxy)-phenyl]-amide (55)

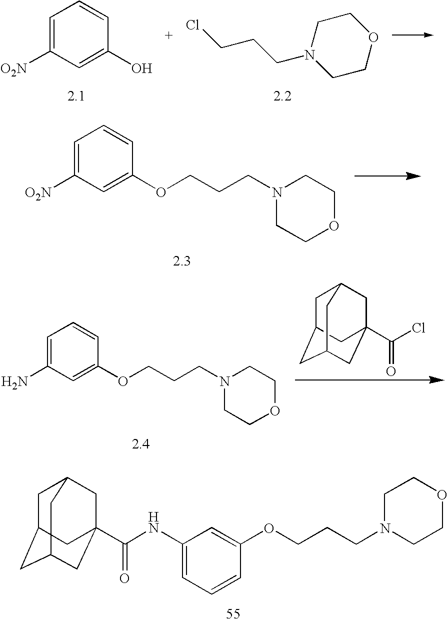

To a stirred solution of m-nitrophenol 2.1 (11.0 g, 79.1 mmol) in dry DMF was added potassium carbonate (21.9 g, 158 mmol), and the resulting mixture was stirred at room temperature for 10 min. The 3-chloropropyl morpholine 2.2 was then added, and the temperature of the reaction mixture was slowly raised to 80° C. and maintained for 6 h. The progress of the reaction was monitored by TLC. On completion of the reaction; the reaction mixture was partitioned between ethyl acetate and water. The organic layer was dried over sodium sulfate, and the solvent was removed under reduced pressure to give the crude nitro intermediate 2.3 which was purified by 10% ether-hexane washings. Yield: 17g.

To a stirred solution of the nitro compound 2.3 (170 g, 63.9 mmol) in 250 mL of toluene and 250 mL of water was added iron powder (17.0 g, 320 mmol) and ammonium formate (13 g, 80 mmol) and the resulting mixture was heated at 80° C. for 4-6 h. After completion of the reaction, the reaction mixture was filtered through celite, washed with hot ethyl acetate and finally partitioned between ethyl acetate and water. The organic phase was dried over anhydrous sodium sulfate and concentrated under reduced pressure. The crude product was purified by chromatography on silica gel (100-200 mesh) eluting with 20% ethyl acetate in hexane to afford 15 g of intermediate 2.4.

To a stirred solution of 3-(3-morpholinopropoxy)benzenamine 2.4 (0.200 g, 0.847 mmol) in dry dichloromethane and triethyl amine (0.251 g, 2.54 mmol) at 0-5° C. under a nitrogen atmosphere was added adamantyl carbonyl chloride (0.168 g, 0.841 mmol) and the resulting mixture was stirred for 30 min. The progress of the reaction was monitored by TLC. On completion of the reaction; the reaction mixture was partitioned between DCM and water. The organic layer was dried over sodium sulfate, and the solvent was removed under reduced pressure to give a crude product which was purified by chromatography on silica gel (100-200 mesh) eluting with 5% ethyl acetate in hexane to afford 210 mg of the title compound 55. ¹HNMR (CDCl₃): δ 7.65 (s, 1H); 7.20-7.15 (m, 1H); 6.90-6.85 (m, 1H); 6.70-6.65 (m, 1H); 4.15-4.05 (m, 2H); 3.70-3.65 (m, 4H); 2.60-2.40 (m, 6H); 2.2-1.65 (m, 17H). Mass: (M+1, 399, 100%). m.p. 160-164° C.

### Example 34

### 2-(Adamantan-1-ylamino)-N-[3-(3-morpholin-4-yl-propoxy)-phenyl]-acetamide (58)

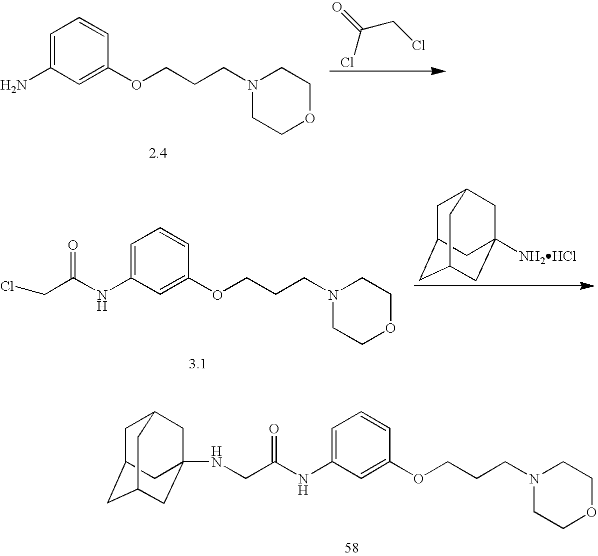

To a stirred solution the 3-(3-morpholinopropoxy)benzenamine 2.4 (2.00 g, 8.47 mmol, prepared as in Example 33) in dry dichloromethane and triethylamine (3.25 g, 32.2 mmol) at 0-5° C. under nitrogen atmosphere was added chloroacetyl chloride (1.67 mL, 14.8 mmol), and the resulting mixture was stirred for 30 min. The progress of the reaction was monitored by TLC. On completion of the reaction, the reaction mixture was partitioned between DCM and water. The organic layer was dried over sodium sulfate, and the solvent was removed under reduced pressure to give 1.52 g of crude intermediate 3.1. To a stirred solution of the adamantylamine hydrochloride (0.144 g, 0.96 mmol) in dry DMF and potassium carbonate (0.176 mg, 1.28 mmol) was added 3.1 (0.200 g, 0.64 mmol) at room temperature, and the temperature of the reaction mixture was raised to 80° C. and maintained for 12 h. The progress of the reaction was monitored by TLC. On completion of the reaction, the reaction mixture was partitioned between ethyl acetate and water. The organic layer was dried over sodium sulfate, and the solvent was removed under reduced pressure to give the crude compound which was purified by column silica gel (100-200 mesh) eluting with 5% ethyl acetate in hexane to afford 196 mg of the title compound 58. ¹HNMR (CDCl₃): δ 9.5 (bs, 1H); 7.40 (s, 1H); 7.25-7.10 (m, 1H); 6.90-6.85 (m, 1H); 6.70-6.65 (m, 1H); 4.15-4.05 (2H, m); 3.70-3.65 (m, 4H); 3.40 (s, 2H); 2.4-2.6 (m, 6H). Mass: (M+1, 428, 100%).

### Example 35

### Adamantane-1-carboxylic acid [3-(3-morpholin-4-yl-propoxy)-phenyl]-amide (56)

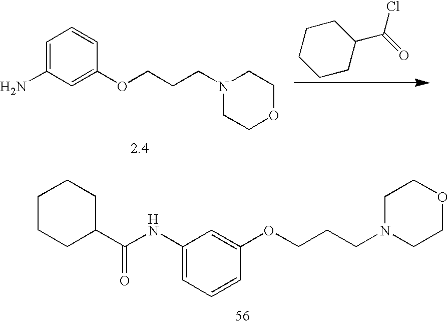

To a stirred solution of the 3-(3-morpholinopropoxy)benzenamine 2.4 (0.200 g, 0.847 mmol) in dry dichloromethane and triethyl amine (0.251 g, 2.542 mmol) at 0-5° C. under nitrogen atmosphere was added cyclohexylcarbonyl chloride (0.168 g, 0.841 mmol) and stirred for 30 min. The progress of the reaction was monitored by TLC. On completion of the reaction, the reaction mixture was partitioned between DCM and water. The organic layer was dried over sodium sulfate, and the solvent was removed under reduced pressure to give the crude which was purified by chromatography on silica gel (100-200 mesh) eluting with 25% ethyl acetate in hexane to afford 160 mg of the title compound 56. ¹HNMR (CDCl₃): δ7.65 (s, 1H); 7.15-7.10 (m, 1H); 6.90-6.85 (d, 1H); 6.70-6.65 (d, 1H); 4.15-4.05 (2H, m); 3.70-3.65 (m, 4H); 2.4-2.6 (m, 6H); 2.20-1.65 (m, 13H). Mass: (M+1, 346,100%).

### Example 36

### N-[3-(3-Morpholin-4-yl-propoxy)-phenyl]-4-trifluoromethyl-benzamide (36)

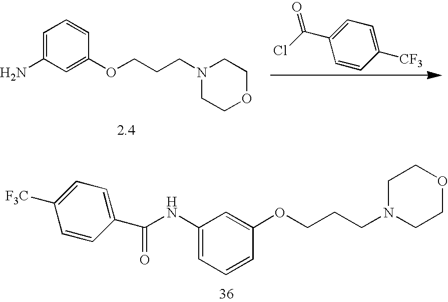

To a stirred solution of the 3-(3-morpholinopropoxy)benzenamine 2.4 (0.200 g, 0.847 mmol) in dry dichloromethane and triethyl amine (0.251 g, 2.54 mmol) at 0-5° C. under a nitrogen atmosphere was added 4-trifluoromethylphenylcarbonyl chloride (0.168 g, 0.841 mmol), and the resulting mixture was stirred for 30 min. The progress of the reaction was monitored by TLC (5% MeOH-DCM). On completion of the reaction; the reaction mixture was partitioned between DCM and water. The organic layer was dried over sodium sulfate, and the solvent was removed under reduced pressure to give the crude which was purified by chromatography on silica gel (100-200 mesh) eluting with 25% ethyl acetate in hexane to afford 220 mg of the title compound 36. ¹HNMR (CDCl₃): δ 8.01 (s, 1H); 7.90-7.80 (m, 2H); 7.75-7.70 (m, 2H); 7.65 (s, 1H); 6.90-6.85 (d, 1H, J=9 Hz); 6.70-6.65 (d, 1H, J=9 Hz); 4.15-4.05 (2H, m); 3.70-3.65 (m, 4H); 2.60-2.40 (m, 6H); 1.90-1.75 (m, 2H); Mass: (M+1, 408, 100%).

### Example 37

### General Synthesis of 6.6

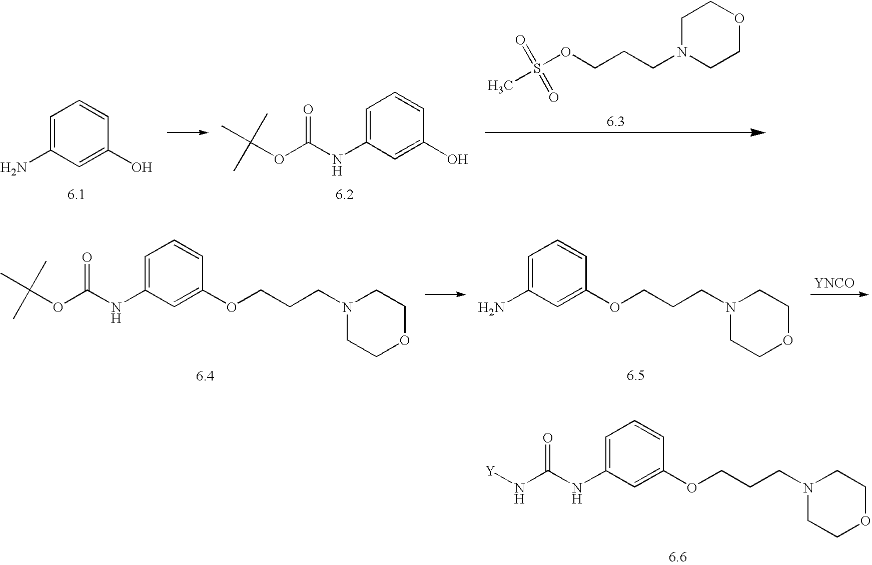

A solution of (Boc)₂O (1.1 equiv.) in THF was added to a stirred solution of 3-aminophenol 6.1 (1 equiv.) in THF. The mixture was heated at reflux for 18 hours. The reaction was followed by TLC which indicated consumption of the starting material and formation of a single product. The solvent was evaporated, and the residue was dissolved in ethyl acetate. The ethyl acetate layer was washed with 1N HCl, saturated NaHCO₃ and brine then dried over sodium sulphate, filtered, and concentrated to afford Boc protected 3-amino phenol 6.2.

To a solution of compound 6.2 (1 equiv.) in DMF (15 volumes) was added cesium carbonate (2 equiv.) followed by a solution of 3-morpholinopropyl methanesulfonate 6.3 (1 equiv.) in DMF. The mixture was stirred at 55° C. for 24 hours then partitioned between ethyl acetate and water. The layers were separated, and the aqueous layer was back extracted with ethyl acetate and combined with the first organic extract. The combined organic extracts were washed with water, 1 M aq. NaH₂PO₄ and brine. The combined NaH₂PO₄ and brine and water layers were back extracted with ethyl acetate. Intermediate 6.4 was then extracted from the ethyl acetate into aqueous 10% H₃PO₄ Concentrated aq. HCl was added to the combined phosphoric acid extracts, and the mixture was stirred at room temperature for 12 hours. The pH of the aqueous solution was adjusted to pH >12 with 6 M aq. NaOH, cooled with ice (100 g), and extracted with DCM. The DCM extracts were dried over sodium sulphate, filtered and concentrated in vacuo to afford compound 6.5.

Amine 6.5 and the appropriate isocyanate YNCO were dissolved in anhydrous DMF and heated to 55° C. overnight. The reaction mixture was allowed to cool to room temperature and then poured into water. The resulting mixture was extracted with ethyl acetate. The combined organic layers were washed with water, dried over sodium sulfate, filtered, and concentrated to afford the desired product 6.6.

### Example 38

### General Synthesis of 7.1

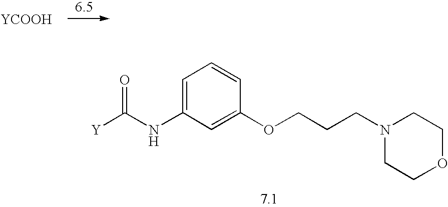

To a solution of the acid YCOOH in DMF was added DMAP and EDC hydrochloride at room temperature. The resulting mixture was stirred for 15 min and then aniline 6.5 was added. The reaction mixture was then stirred overnight. The reaction mixture was poured into water, and the resulting mixture was extracted with DCM. The combined organic extracts were washed with water, 1 N aq. HCl, water, sat. aq. sodium bicarbonate, and finally saturated brine solution. The organic layer was separated, dried over sodium sulfate, filtered and concentrated to give the target molecule.

The following Examples 39-64 were made in a similar fashion to the above examples 37 and 38 or by methods described herein or known to skilled artisans.

### Example 39

### N-[4-(3-Morpholin-4-yl-propoxy)-phenyl]-2-phenyl-acetamide (25)

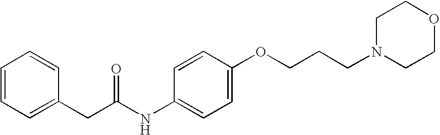

Pale yellow solid; M.P.: 161-165° C.; Mass: 355 [M+1], ¹H NMR (300 MHz; DMSO-d₆); δ 1.8-2.0 (m, 2H, CH₂); 2.4-2.6 (m, 6H, CH₂); 3.6-3.8 (m, 6H, CH₂); 3.8-4.0 (t, 2H, CH₂); 6.6-6.8 (d, 2H, CH₂); 6.9-7.0 (brs, NH); 7.2-7.4 (m, 7H, Ar—CH); LCMS purity: 98.2. %; Yield: 45.5%.

### Example 40

### 1-(4-Chloro-phenyl)-3-[4-(3-morpholin-4-yl-propoxy)-phenyl]-urea (26)

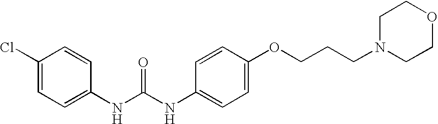

White solid; M.P.: 226-229° C.; Mass: 390 [M+1], ¹H NMR (300 MHz; DMSO-d₆); δ: 2.0-2.2 (m, 2H, CH₂); 1.8-2.0 (d, 2H, CH₂); 4.1-4.3 (m, 2H, CH₂); 4.7-4.9 (m, 2H, CH₂); 3.7 (brs, 2H, CH₂); 3.8-3.9 (m, 1H, CH); 7.6-7.8 (m, 4H, Ar—CH); 8.8 & 9.2 (brs, 2H, NH); LCMS purity: 93.5. %; Yield: 60.2%.

### Example 41

### 2-(4-Chloro-phenyl)-N-[4-(3-morpholin-4-yl-propoxy)-phenyl]-acetamide (20)

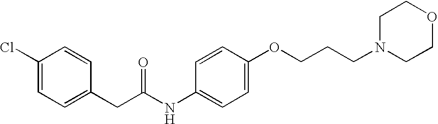

White Solid; M.P.:130-133° C.; Mass: 389 [M+1], ¹H NMR (300 MHz; CDCl₃): δ 1.8-2.0 (m, 2H, CH₂); 2.4-2.6 (m, 6H, CH₂); 3.6-3.8 (m, 6H, CH₂); 3.9-4.1 (t, 2H, CH₂); 6.8-6.9 (d, 2H, CH₂); 7.0 (brs, 1H, NH); 7.2-7.4 (m, 6H, Ar—CH); LCMS purity: 98.5. %; Yield: 50.5%.

### Example 42

### 2-Cyclohexyl-N-[3-(3-morpholin-4-yl-propoxy)-phenyl]-acetamide(51)

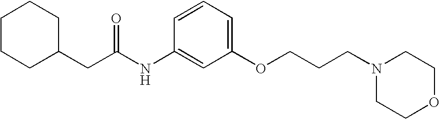

Dark brown liquid; Mass: 361 [M+1], ¹H NMR (300 MHz; CDCl₃): δ 0.9-1.4 (m, 7H, CH2); 1.6-2.0 (m, 10H, CH₂); 2.2-2.3 (d, 2H, CH₂); 2.4-2.6 (m, 6H, CH₂); 3.7-3.9 (t, 4H, CH₂); 4.0-4.2 (t, 2H, CH₂); 6.6-.6.8 (d, 1H, Ar—CH); 6.8-6.9 (t, 1H, Ar—CH); 7.1-7.2 (brs, 2H, ArCH); 7.1-7.2 (1H, NH); 7.4 (s, 1H, Ar—CH); LCMS purity: 98.7. %; Yield: 75%.

### Example 43

### 2-(4-Chloro-phenyl)-N-[3-(3-morpholin-4-yl-propoxy)-phenyl]-acetamide (22)

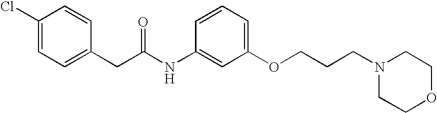

Pale brown solid; Mass: 389 [M+1], M.P.: 125-130° C., ¹H NMR (300 MHz; CDCl₃): δ 2.2-2.4 (m, 2H, CH₂); 2.9-3.3 (m, 6H, CH₂); 3.7-3.8 (s, 2H, CH₂); 4.0-4.2 (m, 6H, CH₂); 6.7 (d, 1H, Ar—CH); 6.9-7.0 (d, 1H, Ar—CH); 7.1-7.2 (t, 1H, Ar—CH); 7.2-7.4 (m, 5H, Ar—CH); 7.5 (brs, 1H, NH); LCMS purity: 97.0. %; Yield: 42.2%.

### Example 44

### 1-Cyclohexyl-3-[4-fluoro-3-(3-morpholin-4-yl-propoxy)-phenyl]-urea (54)

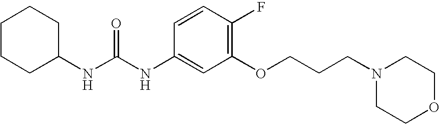

Pale brown solid; Mass: 380 [M+1], MP; 127-131° C., ¹H NMR (300 MHz; CDCl₃): δ 1.0-1.2 (m, 5H, CH₂); 1.2-1.5 (m, 4H, CH₂); 1.9-2.2 (m, 5H, CH₂); 2.4-2.6 (m, 5H, CH₂); 3.4-3.6 (m, 2H, CH₂); 3.6-3.8 (m, 4H, CH₂); 4.0-4.2 (t, 2H, CH); 4.5 (d, 1H, CH); 6.3 (s, 1H, Ar—CH); 6.5 (d, 1H, Ar—CH); 6.9-7.0 (t, 1H, Ar—CH); LCMS purity: 99.5. %; Yield: 50%.

### Example 45

### 1-[4-Fluoro-3-(3-morpholin-4-yl-propoxy)-phenyl]-3-phenyl-urea (29)

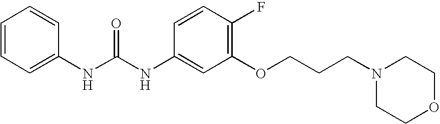

Pale brown solid; Mass: 374 [M+1], MP:179-182° C., ¹H NMR (300 MHz; DMSO-d₆): δ 1.8-2.0 (m, 2H, CH₂); 2.3-2.5 (m, 6H, CH₂); 3.5 (t, 4H, CH₂); 4.0-4.2 (t, 2H, CH₂); 6.8 (m, 1H, Ar—CH); 6.9-7.0 (t, 1H, Ar—CH); 7.1-7.2 (m, 1H, Ar—CH); 7.2-7.3 (t, 2H, Ar—CH); 7.4-7.5 (m, 3H, Ar—CH); LCMS purity:97.9. %; Yield 60%.

### Example 46

### 1-Adamantan-1-yl-3-[4-fluoro-3-(3-morpholin-4-yl-propoxy)-phenyl]-urea (53)

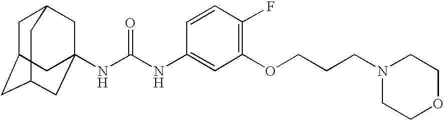

Pale brown solid; Mass: 432 [M+1], M.P.: 189-192° C., ¹H NMR (300 MHz; DMSO-d₆): δ 1.5-1.7 (m, 13H, adamantyl-H); 1.8-2.0 (m, 2H, CH₂); 2.3-2.5 (m, 5H, CH₂); 3.5 (t, 3H, CH₂); 4.0 (t, 2H, CH₂); 5.4 (s, 2H, CH₂); 5.8 (brs, 1H, NH); 6.6 (m, 1H, Ar—CH); 7.1 (t, 1H, Ar—CH); 7.4 (d, 1H, Ar—CH); 8.3 (brs, 1H, NH); LCMS purity: 99.7%; Yield: 45%

### Example 47

### 2-Adamantan-1-yl-N-[4-fluoro-3-(3-morpholin-4-yl-propoxy)-phenyl]-acetamide (52)

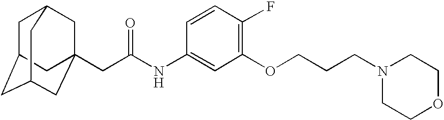

Pale brown solid; Mass: 431 [M+1], MP; 86-90° C.; ¹H NMR (300 MHz; CDCl₃): δ 1.52-1.8 (m, 15H, adamantyl-H); 1.8-2.0 (m, 4H, CH₂); 2.4-2.6 (m, 6H, CH₂); 3.6-3.8 (t, 4H, CH₂); 4.0-4.2 (t, 2H, CH₂); 6.6-6.8 (brs, 1H, NH); 7.0 (m, 2H, Ar—CH); 7.6 (d, 1H, Ar—CH); LCMS purity:95.6%; Yield: 45.2%

### Example 48

### 1-(4-Chloro-phenyl)-3-[4-fluoro-3-(3-morpholin-4-yl-propoxy)-phenyl]-urea (30)

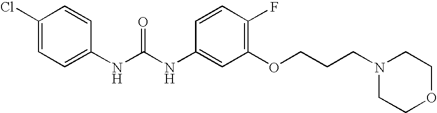

Pale brown solid; Mass: 408 [M+1], M.P.: 204-208° C.; ¹H NMR (300 MHz; CDCl₃; DMSO-d₆): δ 1.8-2.0 (t, 2H, CH₂); 2.4-2.6 (m, 4H, CH₂); 3.3 (s, 2H, CH₂); 3.5-3.7 (t, 4H, v); 4.2 (t, 2H, CH₂); 6.6-6.8 (m, 1H, Ar—CH); 6.8-7.0 (t, 1H, Ar—CH); 7.2 (dd, 2H, Ar—CH); 7.5 (d, 3H, Ar—CH); 8.4 & 8.6 (brs, 2H, NH); LCMS purity: 98.6%; Yield: 45%

### Example 49

### 1-[4-Fluoro-3-(3-morpholin-4-yl-propoxy)-phenyl]-3-(4-fluoro-phenyl)-urea (31)

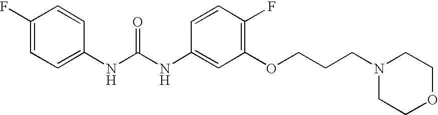

Pale brown solid; Mass: 392 [M+1]; M.P.: 190-194° C.; ¹H NMR (300 MHz; CDCl₃; DMSO-d₆): δ 1.9-2.1 (m, 2H, CH₂); 2.4-2.7 (m, 3H, CH₂); 2.9-3.0 (s, 3H, CH₂); 3.6-3.8 (t, 4H, CH₂); 4.2 (t, 2H, CH₂); 6.6 (m, 1H, Ar—CH); 6.9-7.1 (m, 3H, Ar—CH); 7.4-7.5 (m, 2H, Ar—CH); 7.6 (s, 1H, Ar—CH); 8.2 (brs, 2H, NH); LCMS purity: 97.9%; Yield: 75%.

### Example 50

### 1-Adamantan-1-yl-3-(3-benzyloxy-phenyl)-urea (57)

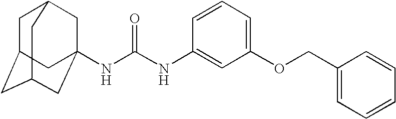

White solid; Mass: 377 [M+1]; M.P.: 232-235° C.; ¹H NMR (300 MHz; CDCl₃): δ 1.6-1.8 (s, 6H, CH₂); 1.9-2.1 (m, 9H, CH₂); 4.6 (brs, 1H, NH); 5.1 (s, 2H, CH₂); 6.2 (brs, 1H, NH); 6.6 (d, Ar—CH); 6.8 (d, 1H, Ar—CH); 7.1 (d, 1H, Ar—CH); 7.2 (t, 1H, Ar—CH); 7.3 (s, 1H, Ar—CH); 7.4-7.5 (m, 5H, Ar—CH); LCMS purity: 99.8%; Yield: 50%.

### Example 51

### 1-[4-(3-Morpholin-4-yl-propoxy)-phenyl]-3-(4-trifluoromethyl-phenyl)-urea (21)

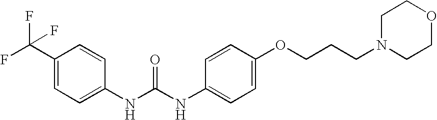

White solid; Mass: 424 [M+1]; M.P.: 213-217° C.; ¹H NMR (300 MHz; DMSO-d₆): δ 2.2-2.4 (m, 2H, CH); 3.0-3.6 (m, 6H, CH₂); 4.1-4.3 (m, 6H, CH₂); 6.8 (d, 2H, Ar—CH); 7.4 (d, 2H, Ar—CH); 7.6 (d, 2H, Ar—CH); 7.7 (d, 2H, Ar—CH); 8.4 & 8.8 (brs, 2H, NH); LCMS purity: 98.3%; Yield: 65%.

### Example 52

### N-[4-(3-Morpholin-4-yl-propoxy)-phenyl]-2-(4-trifluoromethyl-phenyl)-acetamide (27)

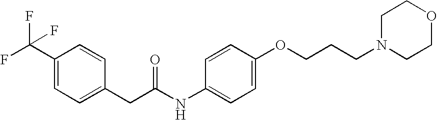

White solid; Mass: 423 [M+1]; M.P.: 106-111° C.; ¹H NMR (300 MHz; CDCl₃): δ 1.8-2.0 (m, 2H, CH₂); 2.4-2.6 (m, 2H, CH₂); 3.6-3.8 (m, 2H, CH₂); 4.0 (t, 2H, CH₂); 6.8 (d, 2H, Ar—CH); 7.0 (brs, 1H, NH); 7.4 (d, 2H, Ar—CH); 7.4-7.58 (d, 2H, Ar—CH); 7.7 (d, 2H, Ar—CH); LCMS purity: 99.1%; Yield: 60.5%.

### Example 53

### 1-[4-(3-Morpholin-4-yl-propoxy)-phenyl]-3-(4-trifluoromethoxy-phenyl)-urea (34)

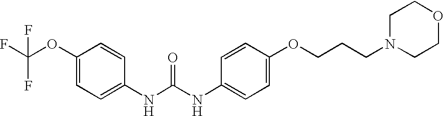

Pale white solid; Mass: 440 [M+1]; M.P.: 155-159° C.; ¹H NMR (300 MHz; CDCl₃): δ 1.8-2.0 (m, 2H, CH₂); 2.4-2.8 (m, 6H, CH₂); 3.8 (t, 4H, CH₂); 4.0 (t, 2H, CH₂); 6.6 & 6.8 (brs, 2H, NH); 6.9 (d, 2H, Ar—CH); 7.2 (d, 1H, Ar—CH); 7.3 (d, 2H, Ar—CH); 7.4 (d, 2H, Ar—CH); LCMS purity: 99.1%; Yield: 50%.

### Example 54

### N-[4-(3-Morpholin-4-yl-propoxy)-phenyl]-2-(4-trifluoromethoxy-phenyl)-acetamide (35)

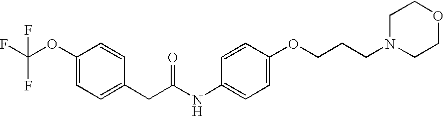

Pale brown solid; Mass: 439 [M+1]; M.P.: 143-146° C.; ¹H NMR (300 MHz; CDCl₃): δ 1.8-2.0 (m, 2H, CH₂); 2.4-2.7 (m, 2H, CH₂); 3.6-3.8 (t, 6H, CH₂); 3.8-4.0 (t, 2H, CH₂); 6.7-6.9 (d, 2H, Ar—CH); 7.0 (brs, 1H, NH); 7.2 (dd, 2H, Ar—CH); 7.4 (m, 4H, Ar—CH); LCMS purity: 88.2%; Yield: 35%.

### Example 55

### 1-[3-(3-Morpholin-4-yl-propoxy)-phenyl]-3-(4-trifluoromethoxy-phenyl)-urea (28)

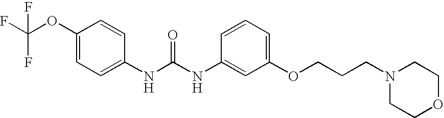

Pale yellow solid; Mass: 440 [M+1]; M.P.: above 300° C.; ¹H NMR (300 MHz; DMSO-d₆): δ 2.1-2.2 (m, 2H, CH₂); 2.9-3.6 (m, 4H, CH₂); 3.6-4.2 (m, 8H, CH₂); 6.5 (d, 1H, Ar—CH); 6.8-7.0 (d, 1H, Ar—CH); 7.1-7.2 (t, 1H, Ar—CH); 7.3 (d, 3H, Ar—CH); 7.6 (d, 2H, CH); 9.0 & 9.2 (brs, 2H, NH); LCMS purity: 95.7%; Yield: 50.5%.

### Example 56

### N-[3-(3-Morpholin-4-yl-propoxy)-phenyl]-2-(4-trifluoromethoxy-phenyl)-acetamide (23)

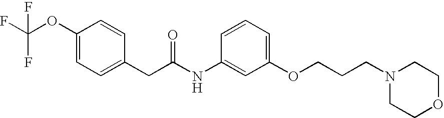

Pale yellow solid; Mass: 439 [M+1]; M.P.: 84-89° C.; ¹H NMR (300 MHz; CDCl₃): δ 1.8-2.0 (m, 2H, CH₂); 2.4-2.6 (m, 6H, CH₂); 3.6-3.8 (m, 6H, CH₂); 4.0 (m, 2H, CH₂); 6.6 (d, 2H, Ar—CH); 6.8 (d, 1H, Ar—CH); 7.0 (brs, 1H, NH); 7.2 (m, 2H, Ar—CH); 7.3 (m, 2H Ar—CH); 7.4 (d, 2H, Ar—CH); LCMS purity: 98.6%; Yield: 60%.

### Example 57

### 1-[4-Fluoro-3-(3-morpholin-4-yl-propoxy)-phenyl]-3-(4-trifluoromethyl-phenyl)-urea (32)

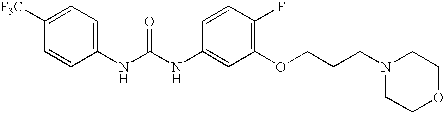

Pale brown solid; Mass: 442 [M+1]; M.P.: 178-182° C.; ¹H NMR (300 MHz; CDCl₃; DMSO-d₆): δ 1.8-2.1 (m, 2H, CH₂); 2.4-2.6 (m, 4H, CH₂); 3.7 (t, 4H, CH₂); 4.2 (t, 2H, CH₂); 6.8 (m, 1H, Ar—CH); 7.0 (m, 1H, Ar—CH); 7.4-7.7 (m, 4H, Ar—CH); 7.8-8.0 (m, 2H, Ar—CH); 8.4 & 8.6 (brs, 2H, NH); LCMS purity: 99.0%; Yield: 45.5%.

### Example 58

### 1-[4-Fluoro-3-(3-morpholin-4-yl-propoxy)-phenyl]-3-(4-trifluoromethoxy-phenyl)-urea (33)

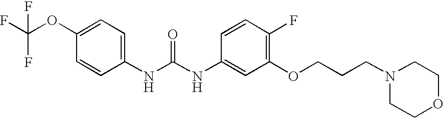

Off white solid; Mass: 458 [M+1]; M.P.: 181-185° C.; ¹H NMR (300 MHz; DMSO-d₆): δ 2.2-2.3 (m, 2H, CH₂); 3.1-3.2 (m, 2H, CH₂); 3.3 (m, 2H, CH₂); 3.4 (m, 2H, CH₂); 3.5 (d, 2H, CH₂); 3.6 (t, 2H, CH₂;) 3.8-4.1 (t, 2 h, CH₂); 6.8 (m, 1H, Ar—CH); 7.2 (t, 1H, Ar—CH); 7.2-7.4 (d, 2H, Ar—CH); 7.4-7.6 (m, 3H, Ar—CH); 8.9 & 9.1 (brs, 2H, NH); LCMS purity: 99.1%; Yield: 55.5%.

### Example 59

### 1-(3-Phenoxy-phenyl)-3-(4-trifluoromethyl-phenyl)-urea (40)

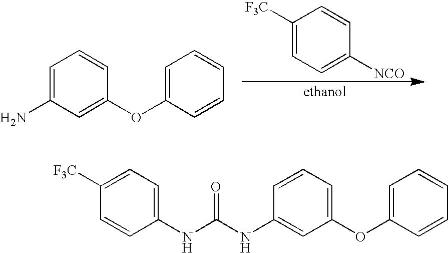

To a stirred suspension of compound 3-phenoxyaniline (1 equivalent) in ethanol was added 1-isocyanato-4-(trifluoromethyl)benzene (1 equivalent). The reaction mixture was stirred at reflux overnight, and the reaction mixture was cooled to room temperature and evaporated in vacuo. The residue was washed first with n-pentane and then with diethyl ether to give compound 40 as an off white solid. Mass: 373 [M+1]; M.P.: 178-181° C.; ¹H NMR (300 MHz; DMSO-d₆): δ 6.6-6.7 (m, 1H, Ar—CH); 7.0 (d, 2H, Ar—CH); 7.1 (m, 2H, Ar—CH); 7.2 (m, 2H, Ar—CH); 7.3-7.4 (t, 2H, Ar—CH); 7.5 (m, 1H, Ar—CH); 7.5-7.6 (m, 2H, Ar—CH); 8.4 & 8.5 (brs, 2H, NH); LCMS purity: 99.1%; Yield: 65.5%. ¹H NMR (400 MHz; CD₃OD) δ: 6.9-7.0 (d, 2H, ArCH); 7.1-7.2 (d, 3H, ArCH); 7.4-7.6 (m, 6H, ArCH); 8.0 (d, 2H, ArCH); Yield: 40%.

### Example 60

### N-[3-(3-Morpholin-4-yl-propoxy)-phenyl]-2-(4-trifluoromethyl-phenyl)-acetamide (24)

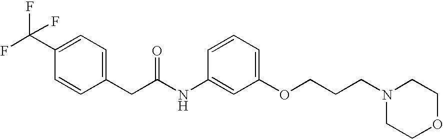

White solid; Mass: 423 [M+1]; M.P.: 105-109° C.; ¹HNMR (300 MHz; CDCl₃): δ 1.8-2.0 (m, 2H, CH₂); 2.4-2.6 (m, 6H, CH₂); 3.6-3.8 (m, 6H, CH₂); 4.0 (t, 2H, CH₂); 6.6 (d, 1H, Ar—CH); 6.8 (d, 1H, Ar—CH); 7.0 (brs, 1H, NH); 7.1 (t, 1H, Ar—CH); 7.3 (s, 1H, Ar—CH); 7.4 (d, 2H, Ar—CH); 7.6 (d, 2H, Ar—CH); LCMS purity: 98.9%; Yield: 45%.

### Example 61

### 1-(4-Phenoxy-phenyl)-3-(4-trifluoromethyl-phenyl)-urea (39)

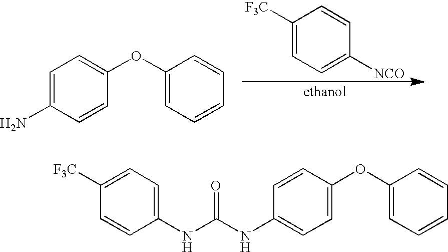

To a stirred suspension of 4-phenoxyaniline (1 equivalent) in ethanol was added 1-isocyanato-4-(trifluoromethyl)benzene (1 equivalent). The reaction mixture was stirred at reflux overnight, and the reaction mixture was cooled to room temperature and evaporated in vacuo. The residue was washed first with n-pentane and then with diethyl ether to give compound 39 as a white solid. Mass: 373 [M+1]; M.P.: 211-213° C.; ¹H NMR (300 MHz; DMSO-d₆): δ 7.0 (q, 4H, Ar—CH); 7.1 (t, 1H, Ar—CH); 7.3-7.4 (t, 2H, Ar—CH); 7.4-7.5 (d, 2H, Ar—CH); 7.6-7.7 (d, 4H, Ar—CH); 8.8 & 9.1 (brs, 2H, NH); LCMS purity: 99.55%; Yield: 45%. Off-white powder; ¹H NMR (400 MHz; CD₃OD) δ: 6.9-7.0 (d, 2H, ArCH); 7.1-7.2 (d, 3H, ArCH); 7.4-7.6 (m, 6H, ArCH); 8.0 (d, 2H, ArCH); Yield: 50%.

### Example 62

### 1-(3-Benzyloxy-phenyl)-3-(4-trifluoromethyl-phenyl)-urea (37)

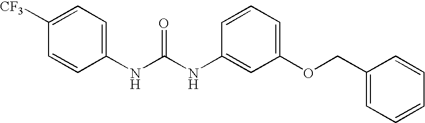

Off-white solid; Mass: 387 [M+1]; M.P.: 208-210° C.; ¹H NMR (300 MHz; DMSO-d₆): δ 5.08, s, 2H); 6.94, 6.74 (m, 1H, NH); 7.15-7.65 (m, 13H, Ar—CH); 8.6, 8.3 (bs, 1H, NH); LCMS purity: 94.8%.

### Example 63

### 3-(4-(3-Adamantan-1-ylureido)phenoxy)benzoic acid (59)

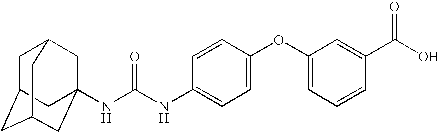

White solid; Mass: 407 [M+1]; M.P.: 185-187° C.; ¹H NMR (300 MHz; DMSO-d₆): δ 1.62 (s, 6H); 1.96 (s, 6H); 2.03 (s, 3H); 5.84 (s, 1H, NH); 6.98 (m, 2H); 7.2-7.62 (m, 6H); 8.33 (s, 1H, NH); 13.0 (bs, 1H); LCMS purity: 99.8%.

### Example 64

### 3-(4-(3-(4-(trifluoromethyl)phenyl)ureido)phenoxy)benzoic acid (60)

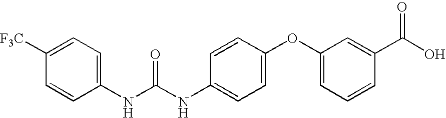

White solid; Mass: 407 [M+1]; M.P.: 308-311° C.; ¹HNMR (300 MHz; DMSO-d₆): δ 7.07 (m, 2H); 7.2-7.8 (m, 10H); 8.85 (s, 1H, NH); 9.13 (s, 1H, NH); 13.0 (bs, 1H); LCMS purity: 98.7%.

### Example 65

### 1-adamantan-1-yl-3-[3-(3-morpholin-4-yl-propoxy)cyclohexyl]thiourea (61)

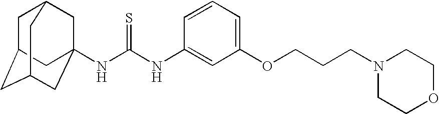

Brown waxy solid; Mass: [M+1] 430; ¹HNMR (CDCl₃): δ 7.7 (bs, 1H); 7.2-7.4 (m, 1H); 6.82-6.97 (m, 3H); 6.2 (bs, 1H); 3.95-4.2 (m, 6H); 3.5-3.7 (m, 2H); 3.2-3.4 (m, 2H); 2.8-3.05 (m, 2H); 2.2-2.4 (m, 11H); 1.6-1.8 (m, 6H); LCMS purity: 90.4%.

### Example 66

### General Synthesis of 9.3

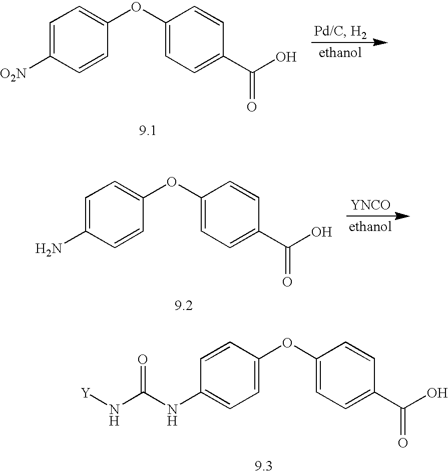

To a stirred solution of compound 9.1 (1 equivalent) in ethanol was added 10% palladium on carbon (0.5 equivalent by weight). The resulting mixture was stirred under hydrogen atmosphere (balloon pressure) overnight. The reaction mixture was filtered through celite and concentrated in vacuo to give compound 9.2 as a solid which was used without purification. The appropriate isocyanate YNCO (1 equivalent) was added to a stirred suspension of compound 9.2 (1 equivalent) in ethanol. The reaction mixture was stirred at reflux overnight, and the reaction mixture was cooled to room temperature and evaporated in vacuo. The residue was washed first with n-pentane and then with diethyl ether to yield the target molecule 9.3.

The following examples 67-70 were made in a similar fashion to the above example 66 or by methods described herein or known to skilled artisans.

### Example 67

### 4-(4-(3-(4-(trifluoromethoxy)phenyl)ureido)phenoxy)benzoic acid (64)

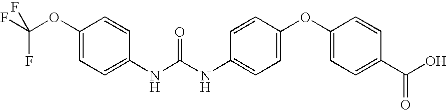

Pale brown powder; Mass 433 [M+1]; M.P.: 299-304° C.; ¹H NMR (400 MHz; DMSO-d₆) δ: 6.9-7.0 (d, 2H, ArCH); 7.1-7.2 (d, 2H, ArCH); 7.3 (d, 1H, ArCH); 7.5-7.6 (m, 4H, ArCH); 7.9-8.0 (d, 2H, ArCH); 8.0-8.1 (s, 1H, ArCH); 8.9-9.0 (s, 1H, NH); 9.0-9.2 (s, 1H, NH); 12.8 (s, 1H, COOH); Yield: 66%.

### Example 68

### 4-(4-(3-(adamantyl)ureido) phenoxy)benzoic acid (65)

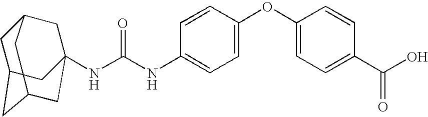

Off-white powder; Mass 407 [M+1]; M.P.: 162-166° C.; ¹H NMR (400 MHz; DMSO-d₆) δ: 1.6-1.8 (s, 6H, CH); 1.8-2.0 (s, 6H, CH); 2.0-2.2 (s, 3H, CH); 5.8-6.0 (s, 1H, NH); 6.8-7.0 (m, 4H, ArCH); 7.4-7.6 (d, 2H, ArCH); 7.8-8.0 (d, 2H, ArCH); 8.2-8.5 (s, 1H, NH); Yield: 70%.

### Example 69

### 4-(4-(3-(4-(trifluoromethyl)phenyl)ureido)phenoxy)benzoic acid (66)

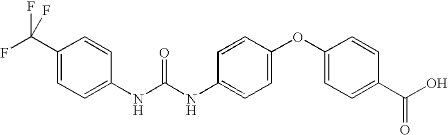

Off-white powder; Mass 417 [M+1]; M.P.: >400° C.; ¹H NMR (400 MHz; CD₃OD) δ: 6.9-7.0 (d, 2H, ArCH); 7.1-7.2 (d, 2H, ArCH); 7.4-7.6 (m, 6H, ArCH); 8.0 (d, 2H, ArCH); Yield: 30%.

### Example 70

### 4-(4-(3-(3-(trifluoromethyl)phenyl)ureido)phenoxy)benzoic acid (67)

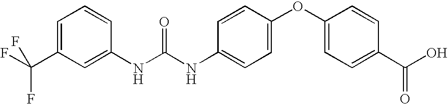

Off-white powder; Mass 417 [M+1]; M.P.: 318° C.; ¹H NMR (400 MHz; DMSO-d₆) δ: 6.9-7.0 (d, 2H, ArCH); 7.1-7.2 (d, 2H, ArCH); 7.3 (d, 1H, ArCH); 7.5-7.6 (m, 4H, ArCH); 7.9-8.0 (d, 2H, ArCH); 8.0-8.1 (s, 1H, Ar—CH); 8.9-9.0 (s, 1H, NH); 9.0-9.2 (s, 1H, NH); 12.8 (s, 1H, COOH); Yield: 62%.

### Example 71

### 3-(3-(3-(adamantyl) ureido)phenoxy)benzoic acid (68)

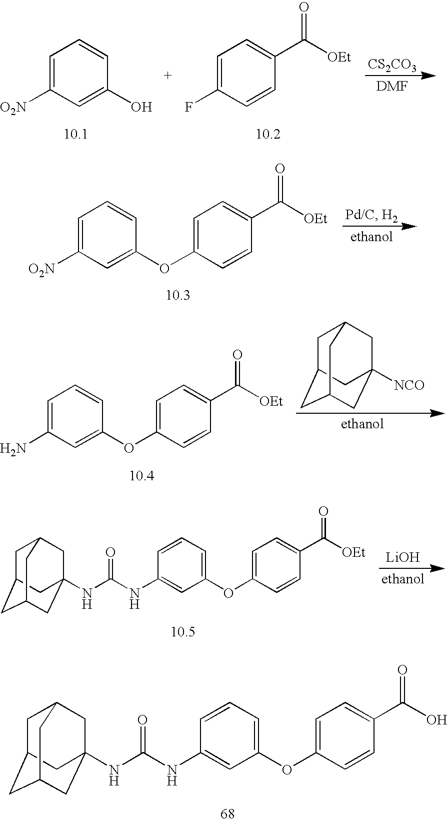

Ethyl 4-fluorobenzoate 10.2 (1 equivalent) was added to a stirred suspension of 3-nitrophenol 10.1 (1 equivalent) and cesium carbonate in dimethyl formamide (DMF) (10 volumes). The reaction mixture was warmed to 100° C. and stirred overnight. The solvent was then removed in vacuo, and the residue was partitioned between water and ethyl acetate. The layers were separated and the organic layer was washed with brine, dried over sodium sulfate, and concentrated in vacuo. The crude product thus obtained was triturated with diethyl ether to give intermediate 10.3 as a solid.

To a stirred solution of intermediate 10.3 (1 equivalent) in ethanol was added 10% palladium on carbon (0.5 equivalent by weight). The resulting mixture was stirred under a hydrogen atmosphere (balloon pressure) overnight. The reaction mixture was filtered through celite and concentrated in vacuo to give ethyl 4-(3-aminophenoxy)benzoate 10.4 as a solid which was used without purification in the next step.

To a stirred suspension of compound 10.4 (1 equivalent) in ethanol was added isocyanatoadamantane (1 equivalent). The reaction mixture was stirred at reflux overnight, then cooled to room temperature and concentrated in vacuo. The residue was washed first with n-pentane and then purified by silica gel column chromatography to give compound 10.5 as a solid.

To a stirred solution of compound 10.5 (1 equivalent) in methanol:tetrahydrofuran:water (9:1:1) was added lithium hydroxide (3 equivalent). The reaction mixture was stirred overnight at room temperature, and the reaction mixture was evaporated in vacuo. The residue was partitioned between water and diethyl ether. The pH of the aqueous layer was adjusted to 2 with 1N HCl and extracted with dichloromethane. The dichloromethane layer was washed with brine, dried over sodium sulfate and concentrated in vacuo to give compound 68 as a light pink powder. M.P: 152-154° C.; Mass 407 [M+1]; ¹H NMR (400 MHz; DMSO-d₆): δ: 1.6-1.8 (s, 6H, CH); 1.8-2.0 (s, 6H, CH); 2.0 (s, 3H, CH); 5.8-6.0 (s, 1H, NH); 6.6 (d, 1H, ArCH); 7.0 (m, 3H, Ar—CH); 7.3 (t, 1H, ArCH); 7.3 (s, 1H, ArCH); 7.8-8.0 (d, 2H, ArCH); 8.5-8.6 (s, 1H, NH); 12.8 (s, 1H, COOH); LCMS purity: 97.9%; Yield: 35%.

### BIOLOGICAL EXAMPLES

### Example 1

### Fluorescent Assay for Mouse and Human Soluble Epoxide Hydrolase

Recombinant mouse sEH (MsEH) and human sEH (HsEH) were produced in a baculovirus expression system as previously reported. Grant et al., J. Biol. Chem., 268:17628-17633 (1993); Beetham et al., Arch. Biochem. Biophys., 305:197-201 (1993). The expressed proteins were purified from cell lysate by affinity chromatography. Wixtrom et al., Anal. Biochem., 169:71-80 (1988). Protein concentration was quantified using the Pierce BCA assay using bovine serum albumin as the calibrating standard. The preparations were at least 97% pure as judged by SDS-PAGE and scanning densitometry. They contained no detectable esterase or glutathione transferase activity which can interfere with the assay. The assay was also evaluated with similar results in crude cell lysates or homogenate of tissues.

The IC₅₀s for each inhibitor were according to the following procedure:

### Substrate:

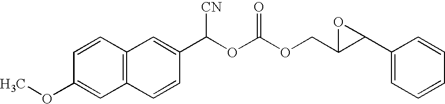

Cyano(2-methoxynaphthalen-6-yl)methyl (3-phenyloxiran-2-yl)methyl carbonate (CMNPC; Jones P. D. et. al.; Analytical Biochemistry 2005; 343: pp. 66-75)

### Solutions:

Bis/Tris HCl 25 mM pH 7.0 containing 0.1 mg/mL of BSA (buffer A) CMNPC at 0.25 mM in DMSO.

Mother solution of enzyme in buffer A (Mouse sEH at 6 μg/mL and Human sEH at 5 μg/mL). Inhibitor dissolved in DMSO at the appropriate concentration.

### Protocol:

In a black 96 well plate, fill all the wells with 150 μL of buffer A.

Add 2 μL of DMSO in well A2 and A3, and then add 2 μL of inhibitor solution in A1 and A4 through A12.

Add 150 L of buffer A in row A, then mix several time and transfer 150 L to row B. Repeat this operation up to row H. The 150 L removed from row H is discarded.

Add 20 L of buffer A in column 1 and 2, then add 20 L of enzyme solution to column 3 to 12.

Incubate the plate for 5 minutes in the plate reader at 30° C.

During incubation prepare the working solution of substrate by mixing 3.68 mL of buffer A (4×0.920 mL) with 266 μL (2×133 μL) of substrate solution).

At t=0, add 30 L of working substrate solution with multi-channel pipette labeled “Briggs 303” and start the reading ([S]final: 5 μM).

Read with ex: 330 nm (20 nm) and em: 465 nm (20 nm) every 30 second for 10 minutes. The velocities are used to analyze and calculate the IC₅₀s.

Table 3 shows the percent inhibition (% Inh) of Compounds 1-61 and 64-75 when tested with the assay at 50, 200, 500, 2000, 5000 nM.

TABLE 3

| | Conc. | |
|---|---|---|
| Cmpd. | (nM) | % Inh |
| 1 | 500 | 62 |
| 2 | 50 | 82 |
| 3 | 50000 | 90 |
| 4 | 50 | 75 |
| 5 | 500 | 78 |
| 6 | 50000 | 92 |
| 7 | 5000 | 87 |
| 8 | 500 | 91 |
| 9 | 50 | 82 |
| 10 | 50 | 74 |
| 11 | 5000 | 85 |
| 12 | 5000 | 87 |
| 13 | 50000 | 75 |
| 14 | 500 | 90 |
| 15 | 50 | 94 |
| 16 | 50000 | 95 |
| 17 | 5000 | 95 |
| 18 | 500 | 66 |
| 19 | 5000 | 92 |
| 20 | 500 | 72 |
| 21 | 50 | 96 |
| 22 | 500 | 73 |
| 23 | 500 | 86 |
| 24 | 500 | 88 |
| 25 | 5000 | 80 |
| 26 | 50 | 88 |
| 27 | 500 | 83 |
| 28 | 50 | 90 |
| 29 | 500 | 73 |
| 30 | 500 | 96 |
| 31 | 500 | 78 |
| 32 | 50 | 87 |
| 33 | 50 | 83 |
| 34 | 50 | 91 |
| 35 | 5000 | 93 |
| 36 | 500 | 79 |
| 37 | 50 | 99 |
| 38 | 50 | 93 |
| 39 | 50 | 90 |
| 40 | 50 | 96 |
| 41 | 50 | 96 |
| 42 | 5000 | 79 |
| 43 | 50 | 93 |
| 44 | 5000 | 70 |
| 45 | 50 | 79 |
| 46 | 50 | 93 |
| 47 | 50 | 99 |
| 48 | 50 | 94 |
| 49 | 50 | 79 |
| 50 | 500 | 85 |
| 51 | 50 | 61 |
| 52 | 500 | 93 |
| 53 | 50 | 92 |
| 54 | 50 | 83 |
| 55 | 5000 | 90 |
| 56 | 5000 | 94 |
| 57 | 50 | 98 |
| 58 | 500 | 81 |
| 59 | 5000 | 99 |
| 60 | 500 | 92 |
| 61 | 500 | 96 |
| 64 | 2000 | 100 |
| 65 | 2000 | 100 |
| 66 | 2000 | 100 |
| 67 | 2000 | 99 |
| 68 | 2000 | 98 |
| 69 | 2000 | 100 |
| 70 | 2000 | 95 |
| 71 | 2000 | 98 |
| 72 | 200 | 89 |
| 73 | 200 | 97 |
| 74 | 2000 | 100 |
| 75 | 2000 | 95 |

### FORMULATION EXAMPLES

The following are representative pharmaceutical formulations containing a compound of the present invention.

### Example 1

### Tablet Formulation

The following ingredients are mixed intimately and pressed into single scored tablets.

| Ingredient | Quantity per tablet, mg |
|---|---|
| Compound of the invention | 400 |
| Cornstarch | 50 |
| Croscarmellose sodium | 25 |
| Lactose | 120 |
| Magnesium stearate | 5 |

### Example 2

### Capsule Formulation

The following ingredients are mixed intimately and loaded into a hard-shell gelatin capsule.

| Ingredient | Quantity per tablet, mg |
|---|---|
| Compound of the invention | 200 |
| Lactose, spray-dried | 148 |
| Magnesium stearate | 2 |

### Example 3

### Suspension Formulation

The following ingredients are mixed to form a suspension for oral administration (q.s.=sufficient amount).

| Ingredient | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| flavoring | 0.035 | mL |
| colorings | 0.5 | mg |

| | |
|---|---|
| distilled water | q.s. to 100 mL |

### Example 4

### Injectable Formulation

The following ingredients are mixed to form an injectable formulation.

| Ingredient | Quantity per tablet, mg |
|---|---|
| Compound of the invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per tablet, mg |
|---|---|
| Compound of the invention | 500 mg |
| Witepsol ® H-15 | balance |

1. A compound of Formula (I′) or a pharmaceutically acceptable salt thereof: 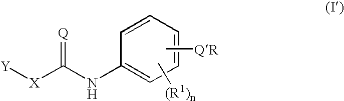
wherein:
Q is O or S;
Q′ is O or S;
R is selected from the group consisting of substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3;
X is selected from the group consisting of a covalent bond, NH, or CR′R″ where R′ and R″ are independently H or alkyl or R′ and R″ together form a C₃-C₆ cycloalkyl ring; and
Y is selected from the group consisting of heteroaryl, substituted heteroaryl, and 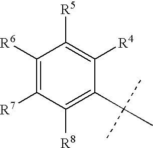
wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring;
provided that
(1) if X is NH and Q is O, then R is not pyridyl, piperidinyl, or piperidinyl substituted with at least one substituent selected from the group consisting of —C(O)H, —C(O)CH₃, —C(O)Oalkyl, —C(O)N(CH₃)₂, dimethylamino, cyanoimino-morpholin-4-yl-methyl, N¹-azetidin-1-yl-N-cyano-amidino, N²-cyano-N¹,N¹-dimethylamidino, N′-cyano-N,N-dimethyl-carbamimidoyl, propionyl, and methylsulfonyl;
(2) if X is NH, Q is O, and Y is methoxyphenyl, then R is not hydroxymethylphenyl, pyridylalkyl, fluoropyridyl, and acetylphenyl;
(3) if Y is pyridyl or substituted pyridyl, then R is alkyl substituted with NR²R³ wherein R² and R³ together form a morpholino or piperazinyl ring;
(4) R is not haloalkyl or mono-substituted alkyl where the substituent is cyano, hydroxyl, or —O—C(O)O-alkyl;
(5) when Y is phenyl, substituted phenyl, heteroaryl or substituted heteroaryl, R is not heteroaryl selected from the group consisting of benzimidazolyl, benzothiazolyl, benzoxazolyl, diazaindolinyl, pyridoimidazolyl, azaindolizinyl, 3,4-diazaindolyl, azaindolyl, 3,4-dihydro-1,4-a,5-triazacarbazolonyl, and 3,4-dihydro-1,4-a-diazacarbazolonyl, wherein the heteroaryl is substituted with at least one substituent selected from the group consisting of amino, (carboxyl ester)amino, acylamino, (substituted sulfonyl)amino, substituted sulfonyl, aminosulfonylamino, and aminocarbonylamino;
and
(6) Formula (I′) is not

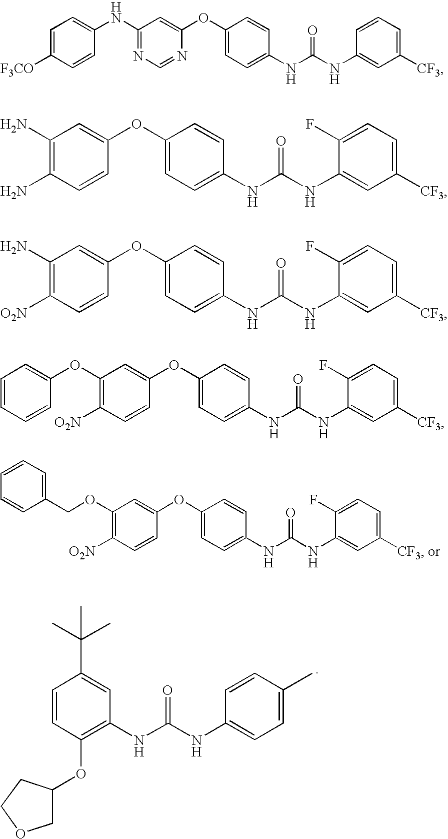

2. A compound of claim 1 of Formula (I) or a pharmaceutically acceptable salt thereof: 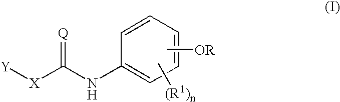
wherein:
Q is O or S;
R is selected from the group consisting of substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3;
X is selected from the group consisting of a covalent bond, NH, or CR′R″ where R′ and R″ are independently H or alkyl or R′ and R″ together form a C₃-C₆ cycloalkyl ring; and
Y is selected from the group consisting of heteroaryl, substituted heteroaryl, and 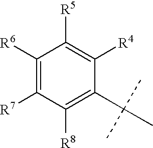
wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring.

3. A compound of claim 2 having Formula (Ia) or (Ib) or a pharmaceutically acceptable salt thereof: 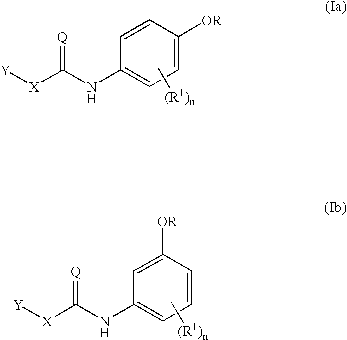
wherein:
Q is O or S;
X is selected from the group consisting of a covalent bond, NH, or CH₂;
R is selected from the group consisting of substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3; and
Y is selected from the group consisting of pyridyl, substituted pyridyl, and 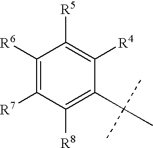
wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring.

4. A compound or salt of claim 3 wherein Q is O.

5. A compound or salt of claim 3 wherein R is substituted alkyl.

6. A compound or salt of claim 5 wherein R is alkyl substituted with aryl, heterocycloalkyl, or substituted heterocycloalkyl.

7. A compound or salt of claim 6 wherein R is alkyl substituted with NR²R³ wherein R² and R³ together form a morpholino or piperazinyl ring, wherein said ring may be substituted or unsubstituted.

8. A compound or salt of claim 3 wherein R is phenyl or substituted phenyl.

9. A compound or salt of claim 3 wherein n is 0.

10. A compound or salt of claim 3 wherein n is 1 and R¹ is halo.

11. A compound or salt of claim 10 wherein R¹ is fluoro.

12. A compound or salt of claim 3 having Formula (Ic) or (Id): 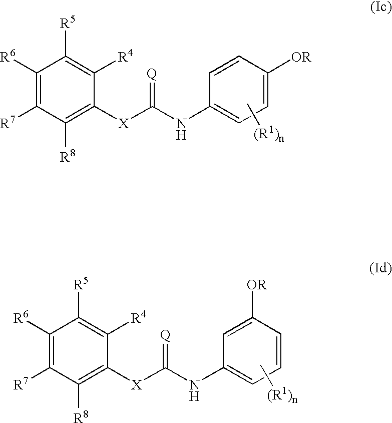
wherein Q, X, n, R¹, R⁴, R⁵, R⁶, R⁷, R⁸, and R are previously defined.

13. A compound or salt of claim 12 wherein Q is O.

14. A compound or salt of claim 12 wherein R is substituted alkyl.

15. A compound or salt of claim 14 wherein R is alkyl substituted with aryl, heterocycloalkyl, or substituted heterocycloalkyl.

16. A compound or salt of claim 15 wherein R is alkyl substituted with NR²R³ wherein R² and R³ together form a morpholino or piperazinyl ring, wherein said ring may be substituted or unsubstituted.

17. A compound or salt of claim 12 wherein R is phenyl or substituted phenyl.

18. A compound or salt of claim 12 wherein n is 0.

19. A compound or salt of claim 12 wherein n is 1 and R¹ is halo.

20. A compound or salt of claim 19 wherein R¹ is fluoro.

21. A compound of claim 12 wherein at least one of R⁴ and R⁸ is fluoro or chloro.

22. A compound of claim 12 wherein R⁵, R⁶, and R⁷ are independently selected from the group consisting of hydrogen, halo, alkyl, haloalkyl, haloalkoxy, alkylamino, heterocycloalkyloxy, alkylthio, haloalkylthio, cyano, alkylsulfonyl, and haloalkylsulfonyl.

23. A compound of claim 22 wherein at least one of R⁵, R⁶, and R⁷ is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl.

24. A compound of claim 23 wherein one of R⁵, R⁶, and R⁷ is selected from the group consisting of halo, alkyl, haloalkyl, haloalkoxy, alkylamino, heterocycloalkyloxy, alkylthio, haloalkylthio, cyano, alkylsulfonyl, and haloalkylsulfonyl, and the remainder of R⁵, R⁶, and R⁷ are hydrogen.

25. A compound of claim 23 wherein at least one of R⁵, R⁶, and R⁷ is selected from the group consisting of halo, trifluoromethyl, trifluoromethoxy, alkylsulfonyl, and haloalkylsulfonyl.

26. A compound of claim 12 wherein R⁶ is selected from the group consisting of chloro, fluoro, trifluoromethyl, and trifluoromethoxy.

27. A compound of claim 26 wherein R⁴, R⁵, R⁷, and R⁸ are hydrogen.

28. A compound of claim 2 or a pharmaceutically acceptable salt thereof selected from Table 1:

| Compound | Structure | Name |
|---|---|---|
| 1 | 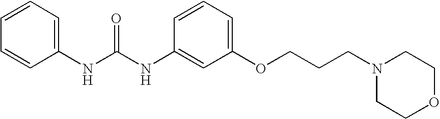 | 1-[3-(3-Morpholin-4-yl-propoxy)-phenyl]-3-phenyl-urea |
| 2 | 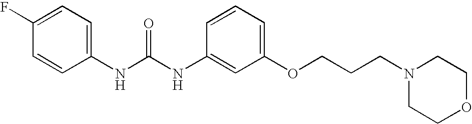 | 1-(4-Fluoro-phenyl)-3-[3-(3-morpholin-4-yl-propoxy)-phenyl]-urea |
| 3 | 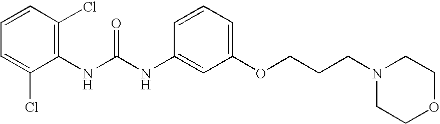 | 1-(2,6-Dichloro-phenyl)-3-[3-(3-morpholin-4-yl-propoxy)-phenyl]-urea |
| 4 | 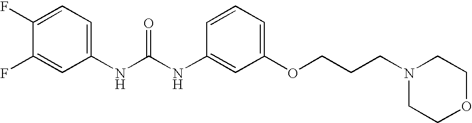 | 1-(3,4-Difluoro-phenyl)-3-[3-(3-morpholin-4-yl-propoxy)-phenyl]-urea |
| 5 | 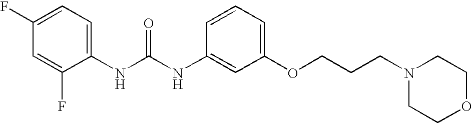 | 1-(2,4-Difluoro-phenyl)-3-[3-(3-morpholin-4-yl-propoxy)-phenyl]-urea |
| 6 | 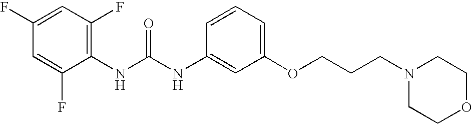 | 1-[3-(3-Morpholin-4-yl-propoxy)-phenyl]-3-(2,4,6-trifluoro-phenyl)-urea |
| 7 | 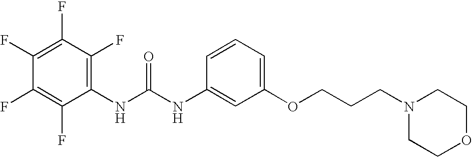 | 1-[3-(3-Morpholin-4-yl-propoxy)-phenyl]-3-pentafluorophenyl-urea |
| 8 | 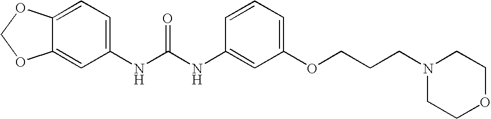 | 1-Benzo[1,3]dioxol-5-yl-3-[3-(3-morpholin-4-yl-propoxy)-phenyl]-urea |
| 9 | 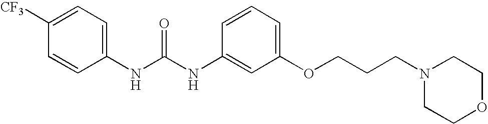 | 1-[3-(3-Morpholin-4-yl-propoxy)-phenyl]-3-(4-trifluoromethyl-phenyl)-urea |
| 10 | 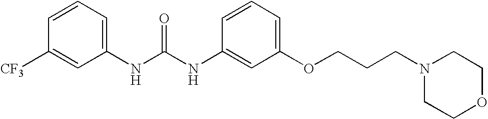 | 1-[3-(3-Morpholin-4-yl-propoxy)-phenyl]-3-(3-trifluoromethyl-phenyl)-urea |
| 11 | 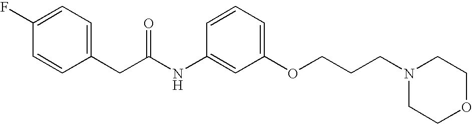 | 2-(4-Fluoro-phenyl)-N-[3-(3-morpholin-4-yl-propoxy)-phenyl]-acetamide |
| 12 | 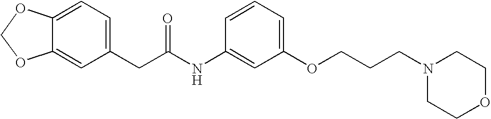 | 2-Benzo[1,3]dioxol-5-yl-N-[3-(3-morpholin-4-yl-propoxy)-phenyl]-acetamide |
| 13 | 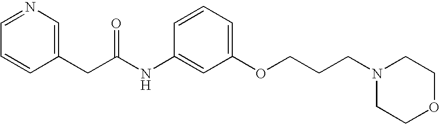 | N-[3-(3-Morpholin-4-yl-propoxy)-phenyl]-2-pyridin-3-yl-acetamide |
| 14 | 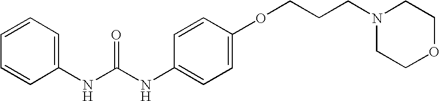 | 1-[4-(3-Morpholin-4-yl-propoxy)-phenyl]-3-phenyl-urea |
| 15 | 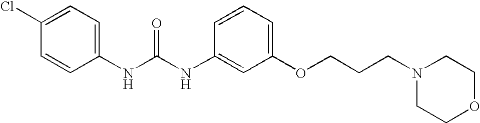 | 1-(4-Chloro-phenyl)-3-[3-(3-morpholin-4-yl-propoxy)-phenyl]-urea |
| 16 | 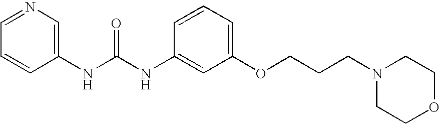 | 1-[3-(3-Morpholin-4-yl-propoxy)-phenyl]-3-pyridin-3-yl-urea |
| 17 | 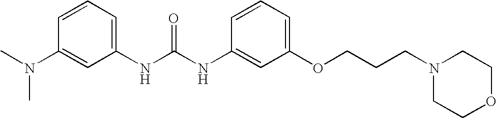 | 1-(3-Dimethylamino-phenyl)-3-[3-(3-morpholin-4-yl-propoxy)-phenyl]-urea |
| 18 | 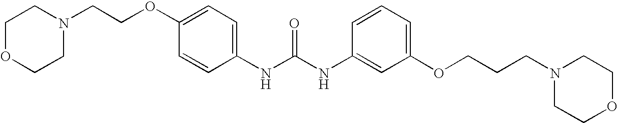 | 1-[4-(2-Morpholin-4-yl-ethoxy)-phenyl]-3-[3-(3-morpholin-4-yl-propoxy)-phenyl]-urea |
| 19 | 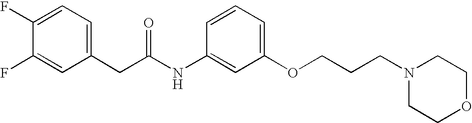 | 2-(3,4-Difluoro-phenyl)-N-[3-(3-morpholin-4-yl-propoxy)-phenyl]-acetamide |
| 20 | 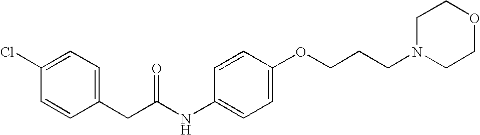 | 2-(4-Chloro-phenyl)-N-[4-(3-morpholin-4-yl-propoxy)-phenyl]-acetamide |
| 21 | 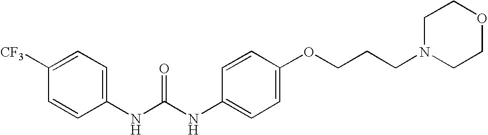 | 1-[4-(3-Morpholin-4-yl-propoxy)-phenyl]-3-(4-trifluoromethyl-phenyl)-urea |
| 22 | 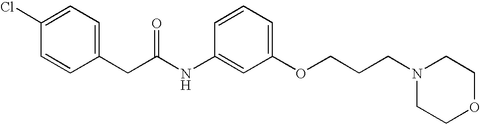 | 2-(4-Chloro-phenyl)-N-[3-(3-morpholin-4-yl-propoxy)-phenyl]-acetamide |
| 23 | 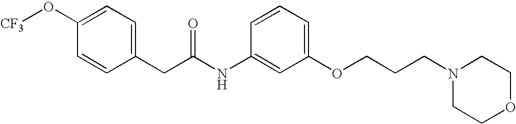 | N-[3-(3-Morpholin-4-yl-propoxy)-phenyl]-2-(4-trifluoromethoxy-phenyl)-acetamide |
| 24 | 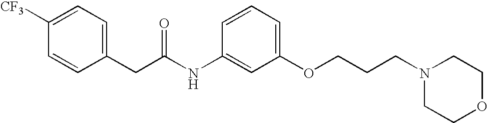 | N-[3-(3-Morpholin-4-yl-propoxy)-phenyl]-2-(4-trifluoromethyl-phenyl)-acetamide |
| 25 | 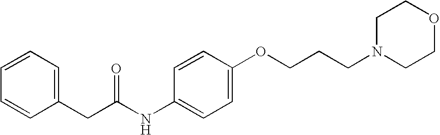 | N-[4-(3-Morpholin-4-yl-propoxy)-phenyl]-2-phenyl-acetamide |
| 26 | 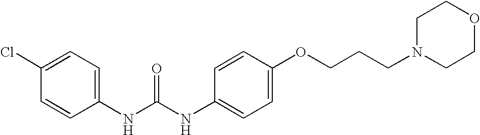 | 1-(4-Chloro-phenyl)-3-[4-(3-morpholin-4-yl-propoxy)-phenyl]-urea |
| 27 | 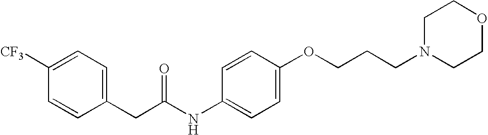 | N-[4-(3-Morpholin-4-yl-propoxy)-phenyl]-2-(4-trifluoromethyl-phenyl)-acetamide |
| 28 | 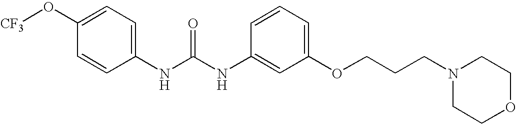 | 1-[3-(3-Morpholin-4-yl-propoxy)-phenyl]-3-(4-trifluoromethoxy-phenyl)-urea |
| 29 | 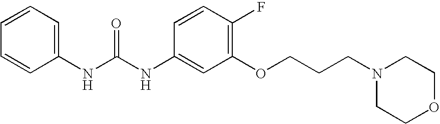 | 1-[4-Fluoro-3-(3-morpholin-4-yl-propoxy)-phenyl]-3-phenyl-urea |
| 30 | 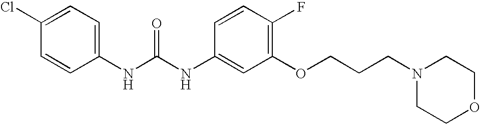 | 1-(4-Chloro-phenyl)-3-[4-fluoro-3-(3-morpholin-4-yl-propoxy)-phenyl]-urea |
| 31 | 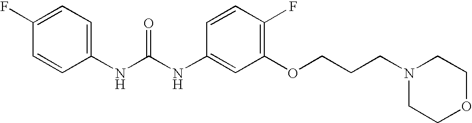 | 1-[4-Fluoro-3-(3-morpholin-4-yl-propoxy)-phenyl]-3-(4-fluoro-phenyl)-urea |
| 32 | 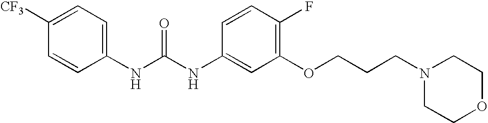 | 1-[4-Fluoro-3-(3-morpholin-4-yl-propoxy)-phenyl]-3-(4-trifluoromethyl-phenyl)-urea |
| 33 | 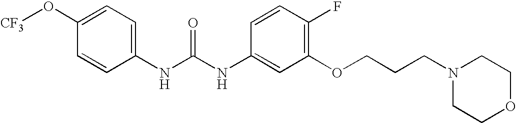 | 1-[4-Fluoro-3-(3-morpholin-4-yl-propoxy)-phenyl]-3-(4-trifluoromethoxy-phenyl)-urea |
| 34 | 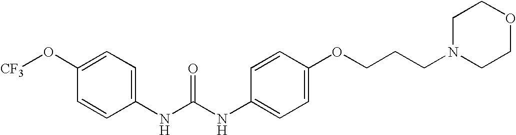 | 1-[4-(3-Morpholin-4-yl-propoxy)-phenyl]-3-(4-trifluoromethoxy-phenyl)-urea |
| 35 | 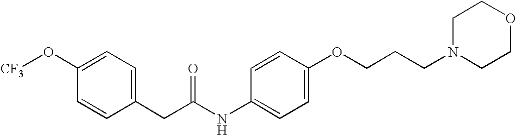 | N-[4-(3-Morpholin-4-yl-propoxy)-phenyl]-2-(4-trifluoromethoxy-phenyl)-acetamide |
| 36 | 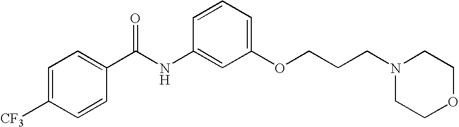 | N-[3-(3-Morpholin-4-yl-propoxy)-phenyl]-4-trifluoromethyl-benzamide |
| 37 | 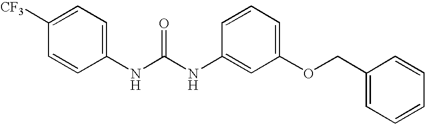 | 1-(3-Benzyloxy-phenyl)-3-(4-trifluoromethyl-phenyl)-urea |
| 38 | 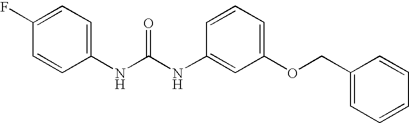 | 1-(3-Benzyloxy-phenyl)-3-(4-fluoro-phenyl)-urea |
| 39 | 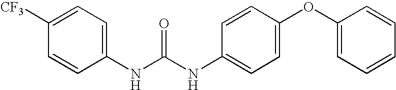 | 1-(4-Phenoxy-phenyl)-3-(4-trifluoromethyl-phenyl)-urea |
| 40 | 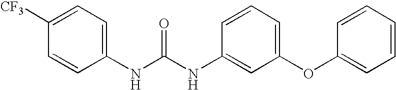 | 1-(3-Phenoxy-phenyl)-3-(4-trifluoromethyl-phenyl)-urea |
| 60 | 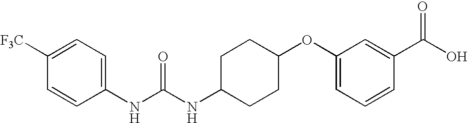 | 3-(4-(3-(4-(trifluoromethyl)phenyl)ureido)phenoxy)benzoicacid |
| 64 | 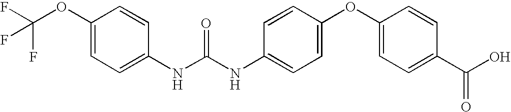 | 4-(4-(3-(4-(trifluoromethoxy)phenyl)ureido)phenoxy)benzoic acid |
| 66 | 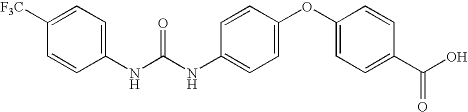 | 4-(4-(3-(4-(trifluoromethyl)phenyl)ureido)phenoxy)benzoicacid |
| 67 | 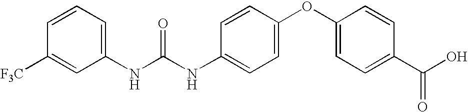 | 4-(4-(3-(3-(trifluoro-methyl)phenyl)ureido)phenoxy)benzoic acid |
| 69 | 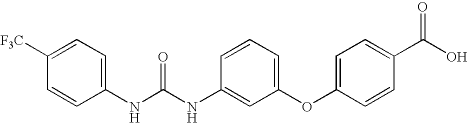 | 4-(3-(3-(4-(trifluoromethyl)phenyl)ureido)phenoxy)benzoicacid |
| 71 | 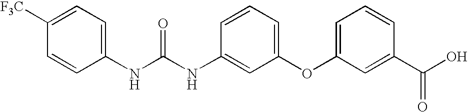 | 3-(3-(3-(4-(trifluoromethyl)phenyl)ureido)phenoxy)benzoicacid |

29. A pharmaceutical composition comprising a pharmaceutically acceptable carrier and a therapeutically effective amount of a compound of claim 1 or a pharmaceutically acceptable salt thereof.

30-31. (canceled)

32. A method for treating a soluble expoxide hydrolase mediated disease, said method comprising administering to a patient a compound of Formula (II) or a pharmaceutically acceptable salt thereof: 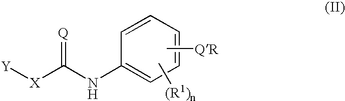
wherein:
Q is O or S;
Q′ is O or S;
R is selected from the group consisting of alkyl, substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3;
X is selected from the group consisting of a covalent bond, NH, or CR′R″ where R′ and R″ are independently H or alkyl or R′ and R″ together form a C₃-C₆ cycloalkyl ring; and
Y is selected from the group consisting of heteroaryl, substituted heteroaryl, and 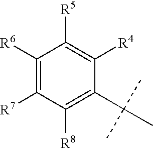
wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring.

33. A method for treating a soluble expoxide hydrolase mediated disease, said method comprising administering to a patient a compound of claim 1 or a pharmaceutically acceptable salt thereof.

34. A method for inhibiting a soluble epoxide hydrolase, comprising contacting the soluble epoxide hydrolase with an effective amount of a compound of Formula (II) or a pharmaceutically acceptable salt thereof: 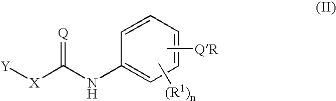
wherein:
Q is O or S;
Q′ is O or S;
R is selected from the group consisting of alkyl, substituted alkyl, aryl, substituted aryl, cycloalkyl, substituted cycloalkyl, cycloalkenyl, substituted cycloalkenyl, heteroaryl, substituted heteroaryl, heterocyclic, and substituted heterocyclic;
each R¹ is independently selected from the group consisting of alkyl, cyano, halo, and haloalkyl;
n is 0, 1, 2, or 3;
X is selected from the group consisting of a covalent bond, NH, or CR′R″ where R′ and R″ are independently H or alkyl or R′ and R″ together form a C₃-C₆ cycloalkyl ring; and
Y is selected from the group consisting of heteroaryl, substituted heteroaryl, and 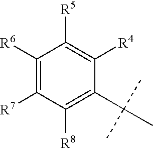
wherein R⁴ and R⁸ are independently hydrogen or halo; and
R⁵, R⁶, and R⁷ are independently selected from the group consisting of hydrogen, halo, alkyl, acyl, acyloxy, alkoxy, heterocycloalkyloxy, carboxyl ester, acylamino, alkylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, alkylsulfonyl and haloalkylsulfonyl; or R⁶ and R⁷ together form a heterocycloalkyl ring.

35. A method for inhibiting a soluble epoxide hydrolase, comprising contacting the soluble epoxide hydrolase with an effective amount of a compound of claim 1.

36. The method of any one of claims 32 to 34 wherein the disease is selected from the group consisting of hypertension, inflammation, adult respiratory distress syndrome, diabetic complications, end stage renal disease, Raynaud syndrome, arthritis, obstructive pulmonary disease, interstitial lung disease, and asthma.

37. A compound or a pharmaceutically acceptable salt thereof selected from:

| Compound | Structure | Name |
|---|---|---|
| 41 | 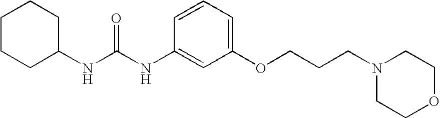 | 1-Cyclohexyl-3-[3-(3-morpholin-4-yl-propoxy)-phenyl]-urea |
| 42 | 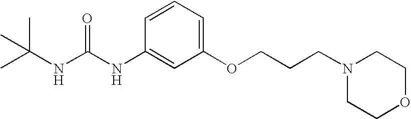 | 1-tert-Butyl-3-[3-(3-morpholin-4-yl-propoxy)-phenyl]-urea |
| 43 | 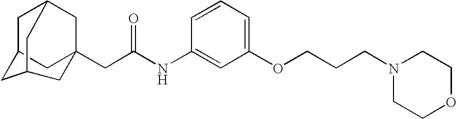 | 2-Adamantan-1-yl-N-[3-(3-morpholin-4-yl-propoxy)-phenyl]-butyramide |
| 44 | 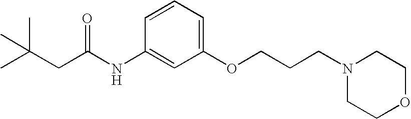 | 3,3-Dimethyl-N-[3-(3-morpholin-4-yl-propoxy)-phenyl]-butyramide |
| 45 | 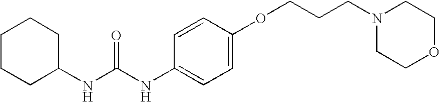 | 1-Cyclohexyl-3-[4-(3-morpholin-4-yl-propoxy)-phenyl]-urea |
| 46 | 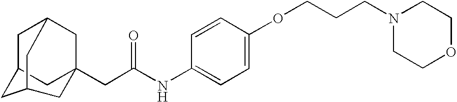 | 2-Adamantan-1-yl-N-[4-(3-morpholin-4-yl-propoxy)-phenyl]-acetamide |
| 47 | 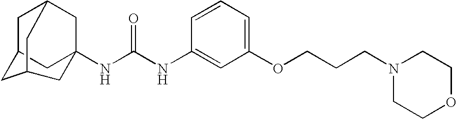 | 1-Adamantan-1-yl-3-[3-(3-morpholin-4-yl-propoxy)-phenyl]-urea |
| 48 | 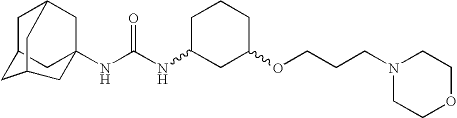 | 1-Adamantan-11-yl-3-[3-(3-morpholin-4-yl-propoxy)-cyclohexyl]-urea |
| 49 | 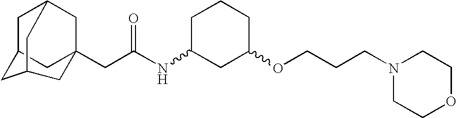 | 2-Adamantan-1-yl-N-[3-(3-morpholin-4-yl-propoxy)-cyclohexyl]-acetamide |
| 50 | 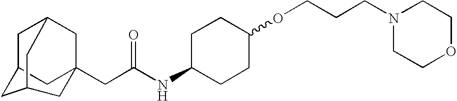 | 2-Adamantan-1-yl-N-[4-(3-morpholin-4-yl-propoxy)-cyclohexyl]-acetamide |
| 51 | 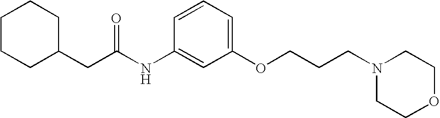 | 2-Cyclohexyl-N-[3-(3-morpholin-4-yl-propoxy)-phenyl]-acetamide |
| 52 | 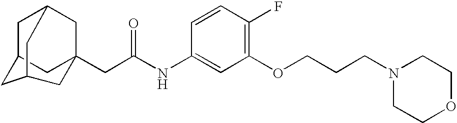 | 2-Adamantan-1-yl-N-[4-fluoro-3-(3-morpholin-4-yl-propoxy)-phenyl]-acetamide |
| 53 | 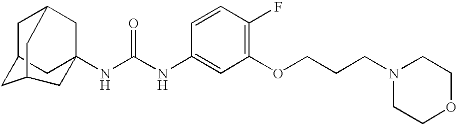 | 1-Adamantan-1-yl-3-[4-fluoro-3-(3-morpholin-4-yl-propoxy)-phenyl]-urea |
| 54 | 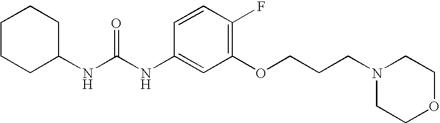 | 1-Cyclohexyl-3-[4-fluoro-3-(3-morpholin-4-yl-propoxy)-phenyl]-urea |
| 55 | 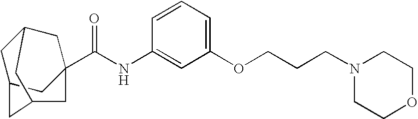 | Adamantane-1-carboxylicacid [3-(3-morpholin-4-yl-propoxy)-phenyl]-amide |
| 56 | 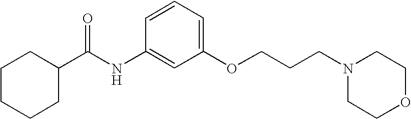 | Cyclohexanecarboxylicacid [3-(3-morpholin-4-yl-propoxy)-phenyl]-amide |
| 57 | 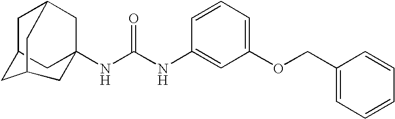 | 1-Adamantan-1-yl-3-(3-benzyloxy-phenyl)-urea |
| 58 | 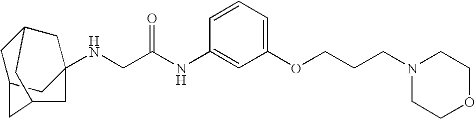 | 2-(Adamantan-1-ylamino)-N-[3-(3-morpholin-4-yl-propoxy)-phenyl]-acetamide |
| 59 | 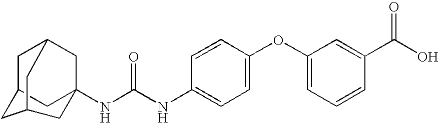 | 3-(4-(3-Adamantan-1-ylureido)phenoxy)benzoicacid |
| 61 | 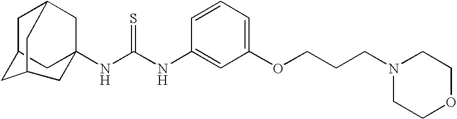 | 1-adamantan-1-yl-3-[3-(3-morpholin-4-yl-propoxy)cyclohexyl]thiourea |
| 62 | 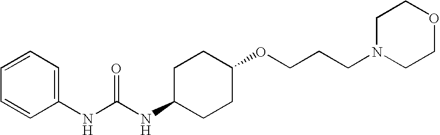 | 1-[4-(3-morpholin-4-yl-propoxy)cyclohexyl]-3-phenylurea |
| 63 | 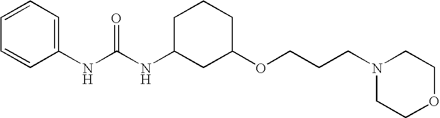 | 1-[3-(3-Morpholin-4-yl-propoxy)cyclohexyl]-3-phenylurea |
| 65 | 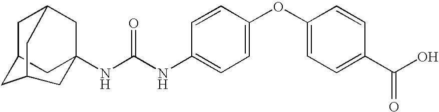 | 4-(4-(3-(adamantanyl)ureido)phenoxy)benzoic acid |
| 68 | 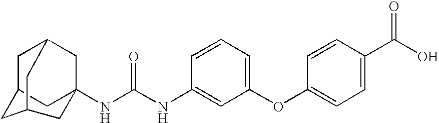 | 4-(3-(3-(adamantanyl)ureido)phenoxy)benzoicacid |
| 70 | 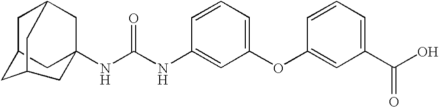 | 3-(3-(3-(adamantanyl)ureido)phenoxy)benzoicacid |
| 72 | 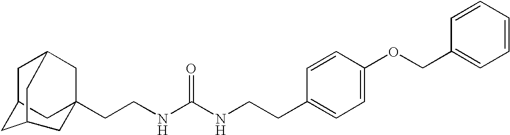 | 1-(4-(benzyloxy)phenethyl)-3-(adamantanylethyl)urea |
| 73 | 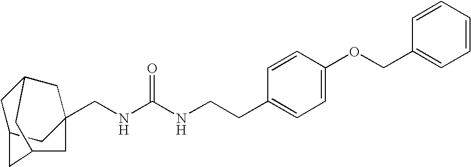 | 1-(4-(benzyloxy)phenethyl)-3-(adamantanylmethyl)urea |

38. A compound or a pharmaceutically acceptable salt thereof, which compound is

| | | |
|---|---|---|
| 74 | 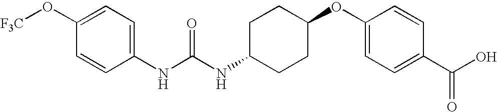 | 4-((1r,4r)-4-(3-(4-(trifluoromethoxy)phenyl)ureido)cyclohexyloxy)benzoic acid |

39. A compound or a pharmaceutically acceptable salt thereof, which compound is

| | | |
|---|---|---|
| 75 | 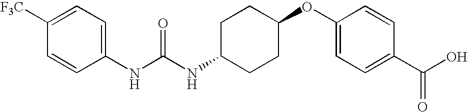 | 4-((1R,4R)-4-(3-(4-(trifluoromethyl)phenyl)ureido)cyclohexyloxy)benzoic acid |

40. A pharmaceutical composition comprising a pharmaceutically acceptable carrier and a therapeutically effective amount of a compound of any one of claims 37-39 or a pharmaceutically acceptable salt thereof.

41. (canceled)

42. A method for treating a soluble expoxide hydrolase mediated disease, said method comprising administering to a patient a compound of any one of claims 37-39 or a pharmaceutically acceptable salt thereof or a pharmaceutical composition of claim 40.

43. A method for inhibiting a soluble epoxide hydrolase, comprising contacting the soluble epoxide hydrolase with an effective amount of a compound of any one of claims 37-39 or a pharmaceutically acceptable salt thereof.

44. The method of claims 42 wherein the disease is selected from the group consisting of hypertension, inflammation, adult respiratory distress syndrome, diabetic complications, end stage renal disease, Raynaud syndrome, arthritis, obstructive pulmonary disease, interstitial lung disease, and asthma.


SEQUENCE LISTING (2 sequences)

SEQ ID NO: 1 - DNA, 2101 bp, Homo sapiens
  CDS (42)..(1703)
ggcacgagctctctctctctctctctctctctctcgccgccatgacgctgcgcggcgccgtcttcgaccttgacggggtgctggcgctgccagcggtgttcggcgtcctcggccgcacggaggaggccctggcgctgcccagaggacttctgaatgatgctttccagaaagggggaccagagggtgccactacccggcttatgaaaggagagatcacactttcccagtggataccactcatggaagaaaactgcaggaagtgctccgagaccgctaaagtctgcctccccaagaatttctccataaaagaaatctttgacaaggcgatttcagccagaaagatcaaccgccccatgctccaggcagctctcatgctcaggaagaaaggattcactactgccatcctcaccaacacctggctggacgaccgtgctgagagagatggcctggcccagctgatgtgtgagctgaagatgcactttgacttcctgatagagtcgtgtcaggtgggaatggtcaaacctgaacctcagatctacaagtttctgctggacaccctgaaggccagccccagtgaggtcgtttttttggatgacatcggggctaatctgaagccagcccgtgacttgggaatggtcaccatcctggtccaggacactgacacggccctgaaagaactggagaaagtgaccggaatccagcttctcaataccccggcccctctgccgacctcttgcaatccaagtgacatgagccatgggtacgtgacagtaaagcccagggtccgtctgcattttgtggagctgggctggcctgctgtgtgcctctgccatggatttcccgagagttggtattcttggaggtaccagatccctgctctggcccaggcaggttaccgggtcctagctatggacatgaaaggctatggagagtcatctgctcctcccgaaatagaagaatattgcatggaagtgttatgtaaggagatggtaaccttcctggataaactgggcctctctcaagcagtgttcattggccatgactggggtggcatgctggtgtggtacatggctctcttctaccccgagagagtgagggcggtggccagtttgaatactcccttcataccagcaaatcccaacatgtcccctttggagagtatcaaagccaacccagtatttgattaccagctctacttccaagaaccaggagtggctgaggctgaactggaacagaacctgagtcggactttcaaaagcctcttcagagcaagcgatgagagtgttttatccatgcataaagtctgtgaagcgggaggactttttgtaaatagcccagaagagcccagcctcagcaggatggtcactgaggaggaaatccagttctatgtgcagcagttcaagaagtctggtttcagaggtcctctaaactggtaccgaaacatggaaaggaactggaagtgggcttgcaaaagcttgggacggaagatcctgattccggccctgatggtcacggcggagaaggacttcgtgctcgttcctcagatgtcccagcacatggaggactggattccccacctgaaaaggggacacattgaggactgtgggcactggacacagatggacaagccaaccgaggtgaatcagatcctcattaagtggctggattctgatgcccggaacccaccggtggtctcaaagatgtagaacgcagcgtagtgcccacgctcagcaggtgtgccatccttccacctgctggggcaccattcttagtatacagaggtggccttacacacatcttgcatggatggcagcattgttctgaaggggtttgcagaaaaaaaagattttctttacataaagtgaatcaaatttgacattattttagatcccagagaaatcaggtgtgattagttctccaggcatgaatgcatcgtccctttatctgtaagaacccttagtgtcctgtagggggacagaatggggtggccaggtggtgatttctctttgaccaatgcatagtttggcagaaaaatcagccgttcatttagaagaatcttagcagagattgggatgccttactcaataaagctaagatgact

SEQ ID NO: 2 - protein, 554 aa, Homo sapiens
MTLRGAVFDLDGVLALPAVFGVLGRTEEALALPRGLLNDAFQKGGPEGATTRLMKGEITLSQWIPLMEENCRKCSETAKVCLPKNFSIKEIFDKAISARKINRPMLQAALMLRKKGFTTAILTNTWLDDRAERDGLAQLMCELKMHFDFLIESCQVGMVKPEPQIYKFLLDTLKASPSEVVFLDDIGANLKPARDLGMVTILVQDTDTALKELEKVTGIQLLNTPAPLPTSCNPSDMSHGYVTVKPRVRLHFVELGWPAVCLCHGFPESWYSWRYQIPALAQAGYRVLAMDMKGYGESSAPPEIEEYCMEVLCKEMVTFLDKLGLSQAVFIGHDWGGMLVWYMALFYPERVRAVASLNTPFIPANPNMSPLESIKANPVFDYQLYFQEPGVAEAELEQNLSRTFKSLFRASDESVLSMHKVCEAGGLFVNSPEEPSLSRMVTEEEIQFYVQQFKKSGFRGPLNWYRNMERNWKWACKSLGRKILIPALMVTAEKDFVLVPQMSQHMEDWIPHLKRGHIEDCGHWTQMDKPTEVNQILIKWLDSDARNPPVVSKM